SEQUENCE LISTING (20 sequences)

SEQ ID NO: 1 - protein, 620 aa, Actinobacillus pleuropneumoniae
MENENKPNVANFEAAVAAKDYEKACSELLLILSQLDSNFGGIHEIEFEYPAQLQDLEQEKIVYFCTRMATAITTLFSDPVLEISDLGVQRFLVYQRWLALIFASSPFVNADHILQTYNREPNRKNSLEIHLDSSKSSLIKFCILYLPESNVNLNLDVMWNISPELCASLCFALQSPRFVGTSTAFNKRATILQWFPRHLDQLKNLNNIPSAISHDVYMHCSYDTSVNKHDVKRALNHVIRRHIESEYGWKDRDVAHIGYRNNKPVMVVLLEHFHSAHSIYRTHSTSMIAAREHFYLIGLGSPSVDQAGQEVFDEFHLVAGDNMKQKLEFIRSVCESNGAAIFYMPSIGMDMTTIFASNTRLAPIQAIALGHPATTHSDFIEYVIVEDDYVGSEECFSETLLRLPKDALPYVPSALAPEKVDYLLRENPEVVNIGIASTTMKLNPYFLEALKAIRDRAKVKVHFHFALGQSNGITHPYVERFIKSYLGDSATAHPHSPYHQYLRILHNCDMMVNPFPFGNTNGIIDMVTLGLVGVCKTGAEVHEHIDEGLFKRLGLPEWLIANTVDEYVERAVRLAENHQERLELRRYIIENNGLNTLFTGDPRPMGQVFLEKLNAFLKEN

SEQ ID NO: 2 - protein, 620 aa, Artificial
  Modified Actinobacillus pleuropneumoniae NGT
MENENKPNVANFEAAVAAKDYEKACSELLLILSQLDSNFGGIHEIEFEYPAQLQDLEQEKIVYFCTRMATAITTLFSDPVLEISDLGVQRFLVYQRWLALIFASSPFVNADHILQTYNREPNRKNSLEIHLDSSKSSLIKFCILYLPESNVNLNLDVMWNISPELCASLCFALQSPRFVGTSTAFNKRATILQWFPRHLDQLKNLNNIPSAISHDVYMHCSYDTSVNKHDVKRALNHVIRRHIESEYGWKDRDVAHIGYRNNKPVMVVLLEHFHSAHSIYRTHSTSMIAAREHFYLIGLGSPSVDQAGQEVFDEFHLVAGDNMKQKLEFIRSVCESNGAAIFYMPSIGMDMTTIFASNTRLAPIQAIALGHPATTHSDFIEYVIVEDDYVGSEECFSETLLRLPKDALPYVPSALAPEKVDYLLRENPEVVNIGIASTTMKLNPYFLEALKAIRDRAKVKVHFHFALGASNGITHPYVERFIKSYLGDSATAHPHSPYHQYLRILHNCDMMVNPFPFGNTNGIIDMVTLGLVGVCKTGAEVHEHIDEGLFKRLGLPEWLIANTVDEYVERAVRLAENHQERLELRRYIIENNGLNTLFTGDPRPMGQVFLEKLNAFLKEN

SEQ ID NO: 3 - protein, 637 aa, Escherichia coli
MMSHKTDTAPVQEQAGLTFRLETFEWQVHQGLNEEAARSLISLLQLLDRHYAQWGESFSAWAPGMTAEEINPHLCTRIAGAITALFSRPGFRVSDGGFAELMDYHRWLAIIFAVSDYRHGDHIIRNINAAGGGVVAPLTLNADNLQLFCLSYYPDSQIALQPEPLWQYDRQTVVRLFFALLSGRALPTPAAHQKREHLLAWLPERLKEIDSLEFLPGKVLHDVYMHCSYADLPEKHRIKQEINRLTARALEQTYADCLPVRAPEAARQKPVLAVVLEWFTCQHSIYRTHSTSMRALREHFHLLGIAQPGATDEITREVFDEFRELSAENVVGDAIRCLSEVRPDVIYYPSVGMFPLTVYLTALRLAPLQLMALGHPATTWSEHIDGVLVEEDYLGDPACFSETVCAVPKDAIPYIPPASTERVLPERTPFRDRAKAAWPAALPVRVAVCASVMKINPGFLDTLREISDRSRVPVQFCFWMGFAQGLTLDYLRRAIRQALPTAEVNAHMPVQAYQQALNSCELFVNPFPFGNTNGLVDTVRQGLPGVCMTGPEVHTHIDEGLFRRLGLPEALIARDREEYITAVLSLTETPRLRERLQKYLTENDVEKVLFEGRPDKFAERVWQLWEARSHRQEEGAE

SEQ ID NO: 4 - protein, 637 aa, Artificial
  Modified Escherichia coli NGT
MMSHKTDTAPVQEQAGLTFRLETFEWQVHQGLNEEAARSLISLLQLLDRHYAQWGESFSAWAPGMTAEEINPHLCTRIAGAITALFSRPGFRVSDGGFAELMDYHRWLAIIFAVSDYRHGDHIIRNINAAGGGVVAPLTLNADNLQLFCLSYYPDSQIALQPEPLWQYDRQTVVRLFFALLSGRALPTPAAHQKREHLLAWLPERLKEIDSLEFLPGKVLHDVYMHCSYADLPEKHRIKQEINRLTARALEQTYADCLPVRAPEAARQKPVLAVVLEWFTCQHSIYRTHSTSMRALREHFHLLGIAQPGATDEITREVFDEFRELSAENVVGDAIRCLSEVRPDVIYYPSVGMFPLTVYLTALRLAPLQLMALGHPATTWSEHIDGVLVEEDYLGDPACFSETVCAVPKDAIPYIPPASTERVLPERTPFRDRAKAAWPAALPVRVAVCASVMKINPGFLDTLREISDRSRVPVQFCFWMGAAQGLTLDYLRRAIRQALPTAEVNAHMPVQAYQQALNSCELFVNPFPFGNTNGLVDTVRQGLPGVCMTGPEVHTHIDEGLFRRLGLPEALIARDREEYITAVLSLTETPRLRERLQKYLTENDVEKVLFEGRPDKFAERVWQLWEARSHRQEEGAE

SEQ ID NO: 5 - protein, 650 aa, Haemophilus influenza
MTKENLQSVPQNTTASLVESNNDQTSLQILKQPPKPNLLRLEQHVAKKDYELACRELMAILEKMDANFGGVHDIEFDAPAQLAYLPEKLLIHFATRLANAITTLFSDPELAISEEGALKMISLQRWLTLIFASSPYVNADHILNKYNINPDSEGGFHLATDNSSIAKFCIFYLPESNVNMSLDALWAGNQQLCASLCFALQSSRFIGTASAFHKRAVVLQWFPKKLAEIANLDELPANILHDVYMHCSYDLAKNKHDVKRPLNELVRKHILTQGWQDRYLYTLGKKDGKPVMMVLLEHFNSGHSIYRTHSTSMIAAREKFYLVGLGHEGVDNIGREVFDEFFEISSNNIMERLFFIRKQCETFQPAVFYMPSIGMDITTIFVSNTRLAPIQAVALGHPATTHSEFIDYVIVEDDYVGSEDCFSETLLRLPKDALPYVPSALAPQKVDYVLRENPEVVNIGIAATTMKLNPEFLLTLQEIRDKAKVKIHFHFALGQSTGLTHPYVKWFIESYLGDDATAHPHAPYHDYLAILRDCDMLLNPFPFGNTNGIIDMVTLGLVGVCKTGDEVHEHIDEGLFKRLGLPEWLIADTRETYIECALRLAENHQERLELRRYIIENNGLQKLFTGDPRPLGKILLKKTNEWKRKHLSKK

SEQ ID NO: 6 - protein, 650 aa, Artificial
  Modified Haemophilus influenza NGT
MTKENLQSVPQNTTASLVESNNDQTSLQILKQPPKPNLLRLEQHVAKKDYELACRELMAILEKMDANFGGVHDIEFDAPAQLAYLPEKLLIHFATRLANAITTLFSDPELAISEEGALKMISLQRWLTLIFASSPYVNADHILNKYNINPDSEGGFHLATDNSSIAKFCIFYLPESNVNMSLDALWAGNQQLCASLCFALQSSRFIGTASAFHKRAVVLQWFPKKLAEIANLDELPANILHDVYMHCSYDLAKNKHDVKRPLNELVRKHILTQGWQDRYLYTLGKKDGKPVMMVLLEHFNSGHSIYRTHSTSMIAAREKFYLVGLGHEGVDNIGREVFDEFFEISSNNIMERLFFIRKQCETFQPAVFYMPSIGMDITTIFVSNTRLAPIQAVALGHPATTHSEFIDYVIVEDDYVGSEDCFSETLLRLPKDALPYVPSALAPQKVDYVLRENPEVVNIGIAATTMKLNPEFLLTLQEIRDKAKVKIHFHFALGASTGLTHPYVKWFIESYLGDDATAHPHAPYHDYLAILRDCDMLLNPFPFGNTNGIIDMVTLGLVGVCKTGDEVHEHIDEGLFKRLGLPEWLIADTRETYIECALRLAENHQERLELRRYIIENNGLQKLFTGDPRPLGKILLKKTNEWKRKHLSKK

SEQ ID NO: 7 - protein, 669 aa, Mannheimia haemolytica
MSAENMPSVIRFEQAVAKKDYESACTELLSILSKLDSNFGGISNIELNMPEQIENLENDKAIYFCTRMAVAITRLFEDPALEISEHGAMRFLTLQRWIALIFASSPYVNADHILRTYNRNKESANPNTVDLDATLQALIKFCILYLPESNILLNLDAAWNASSDLTASLCFALQSPRFIGTSSAFAKRAAILQWFPEKLAQIENLNKLPSAISHDVYMHCSYDIEANKHNVKRSLNAVIRRHLLSVGWEDRKIEQLGTRNNKPVMVVLLEHFHSSHSIYRTHSTSMVAAREHFHLIGLGSDAVDEMGQQVFDEFHLLPQDGSLFDRLSFLKDICDKNNPAVFYMPSIGMDLTTIFASNTRLAPIQAVALGHPATTHSDFIEYVIVEDDYVGSESCFSEQLLRLPKDALPYVPSALAPQNVVYNLRENPEVIHIGIASTTMKLNPYFLEALKAIRDRAKVKTHFHFALGQSSGITHPYVERFIKSYLGNDATAHPHSPYDEYLNILHNCDMMLNPFPFGNTNGIIDMVTLGLVGVCKTGPEVHEHIDEGLFKRLGLPNWLITQTAEEYVTQAIRLAENHEERLAIRRDIIENNKLQTLFSGDPRPMGQIFLAKVQAWLADKNPKNAEVEVKTKKVRKAATASQSAKKQTTSKTQTAKAEKDNAAKTETKS

SEQ ID NO: 8 - protein, 669 aa, Artificial
  Modified Mannheimia haemolytica NGT
MSAENMPSVIRFEQAVAKKDYESACTELLSILSKLDSNFGGISNIELNMPEQIENLENDKAIYFCTRMAVAITRLFEDPALEISEHGAMRFLTLQRWIALIFASSPYVNADHILRTYNRNKESANPNTVDLDATLQALIKFCILYLPESNILLNLDAAWNASSDLTASLCFALQSPRFIGTSSAFAKRAAILQWFPEKLAQIENLNKLPSAISHDVYMHCSYDIEANKHNVKRSLNAVIRRHLLSVGWEDRKIEQLGTRNNKPVMVVLLEHFHSSHSIYRTHSTSMVAAREHFHLIGLGSDAVDEMGQQVFDEFHLLPQDGSLFDRLSFLKDICDKNNPAVFYMPSIGMDLTTIFASNTRLAPIQAVALGHPATTHSDFIEYVIVEDDYVGSESCFSEQLLRLPKDALPYVPSALAPQNVVYNLRENPEVIHIGIASTTMKLNPYFLEALKAIRDRAKVKTHFHFALGASSGITHPYVERFIKSYLGNDATAHPHSPYDEYLNILHNCDMMLNPFPFGNTNGIIDMVTLGLVGVCKTGPEVHEHIDEGLFKRLGLPNWLITQTAEEYVTQAIRLAENHEERLAIRRDIIENNKLQTLFSGDPRPMGQIFLAKVQAWLADKNPKNAEVEVKTKKVRKAATASQSAKKQTTSKTQTAKAEKDNAAKTETKS

SEQ ID NO: 9 - protein, 654 aa, Haemophilus dureyi
MELHSPSLEKFEAAVIEKDYELACTELLAILDKLDNNFGTLQDIEFAYPPQLEDLEQDKVVYFCTRMATVITTLFTDVEFAISSAGAQRFLVFQRWLSFIFASSPFINADHILQSYNCNPDRDIEDDIHLAATKEALIKFCVMYLPESNLKLNLDAAWNVDPELCASLCFALQSPRFLGTVAAYSKRSAILQWFPEHLAQLANLDNIPSAISHDVYMHCSYDIAENKHAVKKALNQVIRRHVVNEYGWQDRDTTRIGYRNDKPVMVVLLEHFHSAHSIYRTHSTSMIAAREHFYLIGLGSKAVDANGQAVFDEFHLLEDDNMKDKLDHIRSICEQNGAAILYMPSVGMDLSTIFVSNTRLAPIQVIALGHPATTYSEFIDYVIVEEDYIGSEACFSETLLPLPKDALPYVPSALAPEKVEYLLRENPEVVNIGIAATTMKLNPYFLDALKVIRDRAKVKIHFHFALGQSTGVTHPHIARFIKSYLGDSATAYPHAPYHQYLTVLHNCDMMLNPFPFGNTNGIIDMVTLGLVGICKTGDEVHEHIDEGLFKRLGLPEWLIADTVDEYIECALRLAENHTERLALRRHIIENNGLATLFTGDPSPMGSVLLAKLNEWREQQKTVAPLKKTKKVAKKATETNKSVTKKPVAKKKRSS

SEQ ID NO: 10 - protein, 654 aa, Artificial
  Modified Haemophilus dureyi NGT
MELHSPSLEKFEAAVIEKDYELACTELLAILDKLDNNFGTLQDIEFAYPPQLEDLEQDKVVYFCTRMATVITTLFTDVEFAISSAGAQRFLVFQRWLSFIFASSPFINADHILQSYNCNPDRDIEDDIHLAATKEALIKFCVMYLPESNLKLNLDAAWNVDPELCASLCFALQSPRFLGTVAAYSKRSAILQWFPEHLAQLANLDNIPSAISHDVYMHCSYDIAENKHAVKKALNQVIRRHVVNEYGWQDRDTTRIGYRNDKPVMVVLLEHFHSAHSIYRTHSTSMIAAREHFYLIGLGSKAVDANGQAVFDEFHLLEDDNMKDKLDHIRSICEQNGAAILYMPSVGMDLSTIFVSNTRLAPIQVIALGHPATTYSEFIDYVIVEEDYIGSEACFSETLLPLPKDALPYVPSALAPEKVEYLLRENPEVVNIGIAATTMKLNPYFLDALKVIRDRAKVKIHFHFALGASTGVTHPHIARFIKSYLGDSATAYPHAPYHQYLTVLHNCDMMLNPFPFGNTNGIIDMVTLGLVGICKTGDEVHEHIDEGLFKRLGLPEWLIADTVDEYIECALRLAENHTERLALRRHIIENNGLATLFTGDPSPMGSVLLAKLNEWREQQKTVAPLKKTKKVAKKATETNKSVTKKPVAKKKRSS

SEQ ID NO: 11 - protein, 690 aa, Bibersteinia trehalosi
MSQEQKTPSVIRFEQAVKAKQYESACNELLDILSQIDSNFGGINGIEFNCPEQLNNPNLSKEKTIYFSTRMADLITELFSDESLSLTVGGAVRFFSYQRWIALLFACSPYINSDHILQVYNRNPDKSNPNSVHLSANPNDLVKFCIMYLPESNISLNLDAIWQLNPTLCASMCFALQSPRFIGTKEAFGKRGAILQWFPEKLAQLPNLDNLPSSISHDVYMHCSYDVAANKHDVKRALNQVMRRHLVTSGWVDRDISKIGKTNGKPVMVVLLEHFHSAHSIYRTHSTSMRAAREHFHLIGIGGSAVDKAGQEVFDDFRLVEGNTIFEKLSFVKRLCEEYGAAIFYMPSIGMDLTTIFASNTRLAPIQAIALGHPGTTHSEFIEYVVVEDDYVGSEACFSEKLLRLPKDALPYVPSALAPASVEYRLRENPEVVNIGIASTTMKLNPYFLDALKAIRDRAKVKVHFHFALGQSSGITHPYVERFIKSHLGDSATAHPHSPYHQYMQILHNCDMLVNPFPFGNTNGIIDMVTLGLVGICKTGPEVHEHIDEGLFKRLGLPEWLIANTVDEYVERAVRLAENHAERLALRRHIIENNGLQTLFTGDPKPMGQVFVQKLNEWAGLHNIDVSDFAFAQSSGKKVTKSAKTAAKKTVKVTVKKSAQPKESTKTKSKTEKKKTSSVKDAAKTSKKKA

SEQ ID NO: 12 - protein, 690 aa, Artificial
  Modified Bibersteinia trehalosi NGT
MSQEQKTPSVIRFEQAVKAKQYESACNELLDILSQIDSNFGGINGIEFNCPEQLNNPNLSKEKTIYFSTRMADLITELFSDESLSLTVGGAVRFFSYQRWIALLFACSPYINSDHILQVYNRNPDKSNPNSVHLSANPNDLVKFCIMYLPESNISLNLDAIWQLNPTLCASMCFALQSPRFIGTKEAFGKRGAILQWFPEKLAQLPNLDNLPSSISHDVYMHCSYDVAANKHDVKRALNQVMRRHLVTSGWVDRDISKIGKTNGKPVMVVLLEHFHSAHSIYRTHSTSMRAAREHFHLIGIGGSAVDKAGQEVFDDFRLVEGNTIFEKLSFVKRLCEEYGAAIFYMPSIGMDLTTIFASNTRLAPIQAIALGHPGTTHSEFIEYVVVEDDYVGSEACFSEKLLRLPKDALPYVPSALAPASVEYRLRENPEVVNIGIASTTMKLNPYFLDALKAIRDRAKVKVHFHFALGASSGITHPYVERFIKSHLGDSATAHPHSPYHQYMQILHNCDMLVNPFPFGNTNGIIDMVTLGLVGICKTGPEVHEHIDEGLFKRLGLPEWLIANTVDEYVERAVRLAENHAERLALRRHIIENNGLQTLFTGDPKPMGQVFVQKLNEWAGLHNIDVSDFAFAQSSGKKVTKSAKTAAKKTVKVTVKKSAQPKESTKTKSKTEKKKTSSVKDAAKTSKKKA

SEQ ID NO: 13 - protein, 621 aa, Aggregatibacter aphrophilus
MSEKKNPSVIQFEKAIREKNYEAACTELLDILNKIDTNFGDIEGIDFDYPQQLKTLMQERIVYFCTRMANAITQLFCDPQFSLSESGANRFFVVQRWLNLIFASSPYINADHILQTYNCNPERDSIYDIYLEPNKNVLMKFAVLYLPESNVNLNLDTMWETDKNICGSLCFALQSPRFIGTPAAFSKRSTILQWFPAKLEQFHVLDDLPSNISHDVYMHCSYDTAENKHNVKKALNQVIRSHLLKCGWQDRQITQIGMRNGKPVMVVVLEHFHSSHSIYRTHSTSMIAAREQFYLIGLGNNAVDQAGRDVFDEFHEFDDSNILKKLAFLKEMCEKNDAAVLYMPSIGMDLATIFVSNARFAPIQVIALGHPATTHSEFIEYVIVEDDYVGSVSCFSETLLRLPKDALPYVPSSLAPTDVQYVLQETPEVVNIGIAATTMKLNPYFLETLKTIRDRAKVKVHFHFALGQSIGITHPYVARFIRSYLGNDATAHPHSPYNRYLDILHNCDMMLNPFPFGNTNGIIDMVTLGLVGVCKTGPEVHEHIDEGLFKRLGLPEWLIADSVEDYIERAIRLAENHQERLALRRHIIENNGLKTLFSGDPSPMGKMLFAKLTEWRQTNGI

SEQ ID NO: 14 - protein, 621 aa, Artificial
  Modified Aggregatibacter aphrophilus NGT
MSEKKNPSVIQFEKAIREKNYEAACTELLDILNKIDTNFGDIEGIDFDYPQQLKTLMQERIVYFCTRMANAITQLFCDPQFSLSESGANRFFVVQRWLNLIFASSPYINADHILQTYNCNPERDSIYDIYLEPNKNVLMKFAVLYLPESNVNLNLDTMWETDKNICGSLCFALQSPRFIGTPAAFSKRSTILQWFPAKLEQFHVLDDLPSNISHDVYMHCSYDTAENKHNVKKALNQVIRSHLLKCGWQDRQITQIGMRNGKPVMVVVLEHFHSSHSIYRTHSTSMIAAREQFYLIGLGNNAVDQAGRDVFDEFHEFDDSNILKKLAFLKEMCEKNDAAVLYMPSIGMDLATIFVSNARFAPIQVIALGHPATTHSEFIEYVIVEDDYVGSVSCFSETLLRLPKDALPYVPSSLAPTDVQYVLQETPEVVNIGIAATTMKLNPYFLETLKTIRDRAKVKVHFHFALGASIGITHPYVARFIRSYLGNDATAHPHSPYNRYLDILHNCDMMLNPFPFGNTNGIIDMVTLGLVGVCKTGPEVHEHIDEGLFKRLGLPEWLIADSVEDYIERAIRLAENHQERLALRRHIIENNGLKTLFSGDPSPMGKMLFAKLTEWRQTNGI

SEQ ID NO: 15 - protein, 619 aa, Yersinia enterocolitica
MVDKTVEVSQEAENLTAFSLPYFEFLVCVRRYEEAGRLLILMLEQLDTQYGRWDVFSLKQQSIQQQEHYCNRLAAAIGNLFSDPGFVLSEKGFLQLINFHRWIALIFAASPFGHADHVITNLNQVGEGCAHPLRFEQNNFLKFCVMYLPESGIPLQPDILWQFNPNAAAALFLALLSPRILPSTVGHAKRELLLRWLPERLLTLDSLEHLPERILHDVYMHCSYADMAEKHAIKRSINFHLRNTLLHNGLSDNHLSPPSRDKPLMLVILEWFNSGHSIYRTHSSTLRAAREQFSTHGATIIDATDAITQAVFDDFTEVNRAGAVEAIVALTQQLLPDVIYFPSVGMFPLTIALTNLRLAPLQVMALGHPATTHSDYIDAVLVEEDYLGDIACFSEKVVSLPKDCLPYVPPANISQPEPILHFAERPAVHIAVCASAMKINPRFLATCAEITRQTSTSVVFHFLVGFCWGITHRVMEKAVNDILPQARVYEHLGYLDYLQVINQCDLFINPFPFGNTNGIVDTVRQGLPGVCLSGTEVHEHIDEGLFRRLGLDEELIAHDLAEYIAVTVRLISDKEWRQSLRQRLLQIQPDNVLFAGKPEQFGLIVRGLLADKKASDKGG

SEQ ID NO: 16 - protein, 619 aa, Artificial
  Modified Yersinia enterocolitica NGT
MVDKTVEVSQEAENLTAFSLPYFEFLVCVRRYEEAGRLLILMLEQLDTQYGRWDVFSLKQQSIQQQEHYCNRLAAAIGNLFSDPGFVLSEKGFLQLINFHRWIALIFAASPFGHADHVITNLNQVGEGCAHPLRFEQNNFLKFCVMYLPESGIPLQPDILWQFNPNAAAALFLALLSPRILPSTVGHAKRELLLRWLPERLLTLDSLEHLPERILHDVYMHCSYADMAEKHAIKRSINFHLRNTLLHNGLSDNHLSPPSRDKPLMLVILEWFNSGHSIYRTHSSTLRAAREQFSTHGATIIDATDAITQAVFDDFTEVNRAGAVEAIVALTQQLLPDVIYFPSVGMFPLTIALTNLRLAPLQVMALGHPATTHSDYIDAVLVEEDYLGDIACFSEKVVSLPKDCLPYVPPANISQPEPILHFAERPAVHIAVCASAMKINPRFLATCAEITRQTSTSVVFHFLVGACWGITHRVMEKAVNDILPQARVYEHLGYLDYLQVINQCDLFINPFPFGNTNGIVDTVRQGLPGVCLSGTEVHEHIDEGLFRRLGLDEELIAHDLAEYIAVTVRLISDKEWRQSLRQRLLQIQPDNVLFAGKPEQFGLIVRGLLADKKASDKGG

SEQ ID NO: 17 - protein, 617 aa, Yersinia pestis
MADKSVELTPVVEAPVVFSLPYFEFLVCTRRYEDAGRLLILMLEKLDTQYGRWDVFSLNKQPIQQQEYYCNRLAAAIGCLFSDPGFVISETGFLQLINFHRWIALIFAASTFGHADHVITNLNEAGNGCSHPLRFERNNFLKFCVMYLPESGIPLQPDILWQFNPQATAALFLALLSPRILPSAAGHEKRETLLAWLPEKLLTLISLEGLPERILHDVYMHCSYADMAKKHTIKRSINFHLRKTMLKNGLSDMNELPPLRSKPLMLVILEWFNSGHSIYRTHSSTLRAARDQFSTHGVAIAEATDDITRKVFDDFTEVSRTGAVETIMALAQQLRPDVIYFPSVGMFPMTVALTNLRLAPLQVMALGHPATTHSDYIDAVLVEEDYLGDIACFSEKVVSLPKDCLPYVPPANITQPEPIQQFVQREAVHIAVCASAMKINPRFLAACAEIALRSPLPIIFHFLVGFCWGITHRVMEKAVNEMVTSAKVYEHLNYQNYLQVINQCDLFINPFPFGNTNGIVDTVRQGLPGVCLSGEEVHEHIDEGLFRRLGLAEELITHNVEQYITATVRLITDTNWRNGLRRQLLQTQPDNVLFTGKPEQFGQIVRALLDNGHQDVN

SEQ ID NO: 18 - protein, 617 aa, Artificial
  Modified Yersinia pestis NGT
MADKSVELTPVVEAPVVFSLPYFEFLVCTRRYEDAGRLLILMLEKLDTQYGRWDVFSLNKQPIQQQEYYCNRLAAAIGCLFSDPGFVISETGFLQLINFHRWIALIFAASTFGHADHVITNLNEAGNGCSHPLRFERNNFLKFCVMYLPESGIPLQPDILWQFNPQATAALFLALLSPRILPSAAGHEKRETLLAWLPEKLLTLISLEGLPERILHDVYMHCSYADMAKKHTIKRSINFHLRKTMLKNGLSDMNELPPLRSKPLMLVILEWFNSGHSIYRTHSSTLRAARDQFSTHGVAIAEATDDITRKVFDDFTEVSRTGAVETIMALAQQLRPDVIYFPSVGMFPMTVALTNLRLAPLQVMALGHPATTHSDYIDAVLVEEDYLGDIACFSEKVVSLPKDCLPYVPPANITQPEPIQQFVQREAVHIAVCASAMKINPRFLAACAEIALRSPLPIIFHFLVGACWGITHRVMEKAVNEMVTSAKVYEHLNYQNYLQVINQCDLFINPFPFGNTNGIVDTVRQGLPGVCLSGEEVHEHIDEGLFRRLGLAEELITHNVEQYITATVRLITDTNWRNGLRRQLLQTQPDNVLFTGKPEQFGQIVRALLDNGHQDVN

SEQ ID NO: 19 - protein, 684 aa, Kingella kingae
MTQTTEQSIPSLTRFEQAVSSQNYEAACTELLSILGQLDSNFGEIHGIEFAYPVQLQNLQQDVTIHFCTRMATAITTLFTNKMWSLTDDGRTRFLTVQRWINMIFASSPYVNADHVLATYNTNPEPDSLWNNIHLDNNQSAFNKFAVMYLPESNVQVNLDSLWSVNPSLTASLCFAWQSPRFIATEAAFNRRAQVLQWFPAKLAQFNNLNTLPANISHDVYMHCSYDIEPNKHDVKGALNQVIRRHILEEYGWQDCDVTKIGNAHGKPVMLVLLEHFHSGHSIYRTHSTSMIAAREQFYLIGIGGAAVDEAGRAVFDEFVEIDAKASTMEKLQAIRAIATKEQPAVFYMPSIGMDLITIFASNTRIAPIQVIALGHPATTHSKFIEYVIVEDDYVGSEECFSETLLRLPKDALPYVPSALAPASVEYNLRENPSVVHIGVASTTMKLNPYFLRACAEIKARSKVPVHFHFAMGQASGVTFAYIERFLKTYLGKAVTAYPHQSYTDYLRTLHQCDMMINPFPFGNTNGIIDMVTLGLVGICKTGAEVHEHIDEGLFKRLGLPEWLITQTADDYVNCAVRLAENHEERLALRRHIIENNGLNTLFSGDPKPMGQILWAKVQEKMAKPAKKATAKVASKPATAVEPVAEKPATKTVRKTASKKAAATEATTEKAAPKTTRTRKKAAE

SEQ ID NO: 20 - protein, 684 aa, Artificial
  Modified Kingella kingae NGT
MTQTTEQSIPSLTRFEQAVSSQNYEAACTELLSILGQLDSNFGEIHGIEFAYPVQLQNLQQDVTIHFCTRMATAITTLFTNKMWSLTDDGRTRFLTVQRWINMIFASSPYVNADHVLATYNTNPEPDSLWNNIHLDNNQSAFNKFAVMYLPESNVQVNLDSLWSVNPSLTASLCFAWQSPRFIATEAAFNRRAQVLQWFPAKLAQFNNLNTLPANISHDVYMHCSYDIEPNKHDVKGALNQVIRRHILEEYGWQDCDVTKIGNAHGKPVMLVLLEHFHSGHSIYRTHSTSMIAAREQFYLIGIGGAAVDEAGRAVFDEFVEIDAKASTMEKLQAIRAIATKEQPAVFYMPSIGMDLITIFASNTRIAPIQVIALGHPATTHSKFIEYVIVEDDYVGSEECFSETLLRLPKDALPYVPSALAPASVEYNLRENPSVVHIGVASTTMKLNPYFLRACAEIKARSKVPVHFHFAMGAASGVTFAYIERFLKTYLGKAVTAYPHQSYTDYLRTLHQCDMMINPFPFGNTNGIIDMVTLGLVGICKTGAEVHEHIDEGLFKRLGLPEWLITQTADDYVNCAVRLAENHEERLALRRHIIENNGLNTLFSGDPKPMGQILWAKVQEKMAKPAKKATAKVASKPATAVEPVAEKPATKTVRKTASKKAAATEATTEKAAPKTTRTRKKAAE

(19) United States
(12) Patent Application Publication
(10) Pub. No.: US 2022/0186276 A1
(43) Pub. Date: Jun. 16, 2022

(54) PLATFORM FOR PRODUCING GLYCOPROTEINS, IDENTIFYING GLYCOSYLATION PATHWAYS

(71) Applicant: Northwestern University, Evanston, IL (US)

(72) Inventors: Michael C. Jewett, Evanston, IL (US); Weston K. Kightlinger, Chicago, IL (US)

(21) Appl. No.: 17/310,191

(22) PCT Filed: Jan. 25, 2019
(86) PCT No.: PCT/US2020/015242

§ 371 Date: Jul. 23, 2021

Related U.S. Application Data
(60) Provisional application No. 62/796,773, filed on Jan. 25, 2019.

(51) Int. Cl. C12P 21/00; C07K 1/107; C12N 1/20

(52) U.S. Cl. CPC C12P 21/005; C12N 1/20; C07K 1/1077

(57) ABSTRACT

Disclosed are components, systems, and methods for glycoprotein protein synthesis in vitro and in vivo. In particular, the disclosed components, systems, and methods relate to modular platforms for producing glycoproteins. The components, systems, and methods disclosed herein may be used in synthesizing glycoproteins and recombinant glycoproteins in cell-free protein synthesis (CFPS) and in modified cells.

### CROSS-REFERENCE TO RELATED APPLIATIONS

The present application claims the benefit of priority under 35 U.S.C. § 119(e) to U.S. Provisional Application No. 62/796,773, filed on Jan. 25, 2019, the content of which is incorporated herein by reference in its entirety.

### STATEMENT REGARDING FEDERALLY SPONSORED RESEARCH OR DEVELOPMENT

This invention was made with government support under HDTRA1-15-1-0052/P00001 awarded by the Defense Threat Reduction Agency. The government has certain rights in the invention.

### BACKGROUND

The present invention generally relates to components, systems, and methods for glycoprotein protein synthesis. In particular, the present invention relates to a modular platform for producing glycoproteins and identifying glycosylation pathways. The components, systems, and methods disclosed herein may be used in synthesizing glycoproteins and recombinant glycoproteins in cell-free protein synthesis (CFPS) and in modified cells.

Glycosylation modulates the pharmacokinetics and potency of protein therapeutics and vaccines. Most methods for glycoprotein synthesis use native pathways within eukaryotic organisms, usually mammalian cells such as Chinese hamster ovary (CHO) cells. However, these methods result in glycan heterogeneity, limit the choice of biomanufacturing hosts, and provide limited control over glycosylation structures which are known to profoundly affect protein properties, especially for protein therapeutics. These limitations have motivated the development of engineered or synthetic glycosylation systems, either by cellular engineering of eukaryotes (typically yeast or CHO cells), bacterial systems, or in vitro. Among these, synthetic glycosylation systems constructed in bacteria or in vitro offer the opportunity to most closely control glycosylation patterns and more rapidly develop more diverse glycosylation patterns. The use of bacterial hosts also enables more cost-effective biomanufacturing.

Several bacterial systems have been developed to produce protein vaccines or glycosylated therapeutics. However, the development of these synthetic glycosylation systems remains slow as it requires the construction and testing sets of enzymes (biosynthetic pathways) in living cells. Consequently, the glycosylation structures produced in bacterial are usually limited to those that can be synthesized by expressing whole operons found in nature, which severely constrains the diversity of structures that can be constructed and therefore the diversity of applications to which this technology can be applied.

Here, the inventors disclose a technology related to a modular cell-free platform for glycosylation pathway assembly by rapid in vitro mixing and expression (GlycoPRIME). Using this technology, the inventors have discovered several novel biosynthetic pathways that can be used for production of glycoprotein therapeutics, vaccines, and analytical standards in vitro or in living cells.

### SUMMARY

Disclosed are components, systems, and methods for glycoprotein protein synthesis in vitro and in vivo. In particular, the disclosed components, systems, and methods relate to modular platforms for producing glycoproteins. The components, systems, and methods disclosed herein may be used in synthesizing glycoproteins and recombinant glycoproteins in cell-free protein synthesis (CFPS) and in modified cells.

The disclosed components, systems, and methods typically include or utilize a soluble or optionally insoluble (e.g., membrane bound) N-linked glycosyltransferase (N-glycosyltransferase, or NGT) to transfer a glucose moiety to a recipient peptide sequence present in a peptide, polypeptide, or protein. The disclosed components, systems, and methods further may include or utilize additional soluble, or optionally insoluble (e.g., membrane bound) glycosyltransferases to modify the N-linked glucose moiety and provide more complex N-linked glycans.

### BRIEF DESCRIPTION OF THE FIGURES

FIG. 1. Provides a diagram for a platform for glycosylation pathway assembly by rapid in vitro mixing and expression (GlycoPRIME). GlycoPRIME was established to construct and screen biosynthetic pathways yielding diverse N-linked glycans. Crude E. coli lysates enriched with a target protein or individual glycosyltransferases (GTs) by cell-free protein synthesis (CFPS) were mixed in various combinations to identify biosynthetic pathways for the construction of various N-linked glycans. A model acceptor protein (Im7-6), the N-linked glycosyltranferase from A. pleuropneumoniae (ApNGT), and 24 elaborating GTs were produced in CFPS and then assembled with activated sugar donors in 37 unique glycosylation pathways. Of these 37 pathways, we identified 23 biosynthetic GT combinations that yield unique glycosylation structures, several with therapeutic relevance. Pathways discovered in vitro were transferred to cell-free or cell-based production platforms to produce therapeutically relevant glycoproteins.

FIG. 2: In vitro synthesis and assembly of one- and two-enzyme glycosylation pathways. (a) Protein name, species, previously characterized activity and optimized soluble CFPS yields for Im7-6 target protein, ApNGT, and GTs selected for glycan elaboration. References for previously characterized activities in FIG. 8. CFPS yields indicate mean and standard deviation (s.d.) from n=3 CFPS reactions quantified by [14C]-leucine incorporation. Full CFPS expression data in FIG. 6 and FIGS. 12, and 13. (b) Symbol key and successful pathways for N-linked glucose installation on Im7-6 by ApNGT and elaboration by selected GTs. Glycan structures herein use Symbol Nomenclature for Glycans (SNFG) and Oxford System conventions for linkages. Sialic acid refers to N-acetylneuraminic acid. (c) Deconvoluted mass spectrometry spectra from Im7-6 protein purified from IVG reactions assembled from CFPS reaction products with and without 0.4 μM ApNGT as well as 2.5 mM UDP-Glc. Full conversion to N-linked glucose was observed after 24 h at 30° C. (d) Intact deconvoluted MS spectra from Im7 protein purified from IVG reactions containing 10 μM Im7-6, 0.4 μM ApNGT, and 7.8 μM NmLgtB, 13.9 μM NgLgtB, 3.1 μM BfGalNAcT, or 9.4 μM Apα1-6. IVG reactions were supplemented with 2.5 mM UDP-Glc as well as 2.5 mM UDP-Gal or 5 mM UDP-GalNAc as appropriate for 24 h at 30° C. Observed mass shifts and MS/MS fragmentation spectra (FIG. 14) are consistent with efficient modification of N-linked glucose with β1-4Gal, β1-4Gal, β1-3GalNAc, or α1-6 dextran polymer. Theoretical protein masses found in FIG. 7. Hpβ4GalT, Btβ4GalT1, and SpWchJ+K did not modify the N-linked glucose installed by ApNGT (FIG. 15). All spectra were acquired from full elution peak areas of all detected glycosylated and aglycosylated Im7-6 species and are representative of n=3 independent IVGs. Spectra from m/z 100-2000 were deconvoluted into 11,000-14,000 Da using Bruker Compass Data Analysis maximum entropy method.

FIG. 3: In vitro synthesis and assembly of complex glycosylation pathways. (a) Protein name, species, previously characterized specificity (FIG. 8), and optimized CFPS soluble yields (FIG. 6) for enzymes tested for elaboration of N-linked lactose. CFPS yields indicate mean and s.d. from n=3 CFPS reactions quantified by [¹⁴C]-leucine incorporation. CjCST-I and HsSIAT1 yields were measured under oxidizing conditions (see FIG. 20). (b) Intact deconvoluted MS spectra from Im7-6 protein purified from IVG reactions with 10 μM Im7-6, 0.4 μM ApNGT, 2 μM NmLgtB, and 2.5 mM appropriate nucleotide-activated sugar donors as well as 4.0 μM BtGGTA, 5.3 μM NmLgtC, 4.9 μM HpFutA, 2.6 μM HpFutC, 4.9 μM PdST6, 5.0 μM CjCST-II, 1.3 μM CjCST-I, 11.5 μM NgLgtA, or 2.2 μM SpPvg1. Mass shifts of intact Im7-6, fragmentation spectra of trypsinized Im7-6 glycopeptides (FIG. 18), and exoglycosidase digestions (FIGS. 21 and 22) are consistent with modification of N-linked lactose with α1-3Gal, α1-4Gal, α1-3 Fuc, α2-6 Sia, α2-3 Sia, α2-8 Sia, β1-3 GlcNAc, or pyruvylation according to known activities of BtGGTA, NmLgtC, HpFutA, HpFutC, PdST6, CjCST-II, CjCST-I, NgLgtA, or SpPvg1. (d) Deconvoluted intact Im7-6 spectra of fucosylated and sialylated LacNAc structures produced by four- and five-enzyme combinations. IVG reactions contained 10 μM Im7-6, 0.4 μM ApNGT, 2 μM NmLgtB, appropriate sugar donors, and indicated GTs at half or one third the concentrations indicated in b for four- and five-enzyme pathways, respectively. Intact mass shifts and fragmentation spectra (FIG. 23) are consistent with fucosylation and sialylation of LacNAc core according to known activities. Intact protein and glycopeptide fragmentation spectra from other screened GTs and GT combinations not shown here are found in FIGS. 17-19 and 23-25. To provide maximum conversion, IVG reactions were incubated for 24 h at 30° C., supplemented with an additional 2.5 mM sugar donors and incubated for another 24 h at 30° C. Spectra were acquired from full elution areas of all detected glycosylated and aglycosylated Im7 species and are representative of n=2 IVGs. Spectra from m/z 100-2000 were deconvoluted into 11,000-14,000 Da using Bruker Compass Data Analysis maximum entropy method.

FIG. 4: Design of biosynthetic pathways for cell-free and bacterial production platforms. (a) One-pot CFPS-GpS for synthesis of H1HA10 protein vaccine modified with αGal glycan. Plasmids encoding the target protein and biosynthetic pathway GTs discovered by GlycoPRIME screening were combined with appropriate activated sugar donors in a CFPS-GpS reaction. (b) Trypsinized glycopeptide MS spectra, (c) exoglycosidase digestions of glycopeptide, and (d) MS/MS glycopeptide fragmentation spectra from H1HA10 purified from IVG reactions containing equimolar amounts of each indicated plasmid encoding H1HA10, ApNGT, NmLgtB, and BtGGTA and 2.5 mM of UDP-Glc and UDP-Gal (see Methods). All reactions contained 10 nM total plasmid concentration and were incubated for 24 h at 30° C. The glycopeptide contains one engineered acceptor sequence located at the N-terminus of H1HA10. Observed masses and mass shifts in b-d spectra are consistent with modification of the H1HA10 peptide with N-linked Glc by ApNGT, lactose (Glcβ1-4Gal) by ApNGT and NmLgtB, or αGal epitope (Glcβ1-4Galα1-3Gal) by ApNGT, NmLgtB, and BtGGTA. (e) Design of cytoplasmic glycosylation systems to produce sialylated IgG Fc in E. coli. Three plasmids containing NmNeuA (CMP-Sia synthesis), IgG Fc engineered with an optimized acceptor sequence (target protein), and biosynthetic pathways discovered using GlycoPRIME (GT operon). (f) Deconvoluted intact glycoprotein MS spectra, (g) exoglycosidase digestions of intact glycoprotein, and (h) MS/MS glycopeptide fragmentation spectra from Fc-6 purified from E. coli cultures supplemented with sialic acid, IPTG, and arabinose and incubated at 25° C. overnight (see Methods). The last GT in all glycosylation pathways is indicated. MS spectra were acquired from full elution areas of all detected glycosylated and aglycosylated protein or peptide species and are representative of n=3 CFPS-GpS or E. coli cultures. MS/MS spectra acquired by pseudo Multiple Reaction Monitoring (MRM) fragmentation at theoretical glycopeptide masses (red diamonds) corresponding to detected intact glycopeptide or protein MS peaks using 30 eV collisional energy. Deconvoluted spectra collected from m/z 100-2000 into 27,000-29,000 Da using Compass Data Analysis maximum entropy method. See FIGS. 9-11 for theoretical masses.

FIG. 5. Provides a table summarizing all of the strains and plasmids used in this study1-6. Plasmid backbone characteristics are listed followed by Uniprot or NCBI identifiers of protein-coding sequences and any modifications or fusion sequences. Annotated protein-coding sequences of all plasmids developed in this study are shown with flanking plasmid sequence contexts in FIG. 29.

FIG. 6. Provides a table showing a summary related to the optimization of cell-free protein synthesis of Im7 target and glycosylation enzymes. CFPS yields of Im7-6 target and enzymes for in vitro glycosylation pathways tested by GlycoPRIME. CFPS yields and errors indicate mean and s.d. from n=3 CFPS reactions quantified by 14C-leucine incorporation. All CFPS reactions were incubated for 20 h at the indicated temperatures and conditions. Solubility was calculated from quantification of yields in fractions isolated after centrifugation at 12,000× g for 15 mins. Asterisk (*) indicates yields when CFPS was conducted under oxidizing conditions. Yields under optimized conditions also shown in FIGS. 2 and 3. Source data underlying listed average and s.d. values are provided in the Source Data file, (available within Kightlinger et al., Nature Communications, 2019, herein incorporated by reference in its entirety).

FIG. 7. Provides a table of theoretical glycoprotein and glycopeptide masses for Im7-6 glycoforms produced during GlycoPRIME biosynthetic pathway engineering. Predicted glycosylation structures are based on previously established GT activities shown in FIGS. 2 and 3 and FIG. 8. Theoretical, neutral, and average masses of expected glycoprotein products as well as theoretical, triply charged, monoisotopic mass-to-charge ratios (m/z) of glycopeptides are shown. Glycopeptide masses correspond to the only ApNGT glycosylation site within Im7-6 which is contained within the tryptic peptide EATTGGNWTTAGGDVLDVLLEHFVK. Experimentally observed masses are annotated in deconvoluted intact protein MS and glycopeptide MS/MS spectra.

FIG. 8. Provides a table showing previously characterized activities of glycosyltransferases used this study7-23. GTs listed below were selected for testing in the GlycoPRIME system based on their previously established activities. Many have also been previously used for biosynthesis of glycolipids or free oligosaccharides, laying the foundation for their testing in the new context of elaborating the N-linked glucose installed by ApNGT in this study.

FIG. 9. Provides a table showing theoretical masses of sugar fragment ions detected in glycopeptide MS/MS spectra. During MS/MS fragmentation of glycopeptides, diagnostic sugar ions were detected. Theoretical mass to charge ratios of these sugar ions are shown in the table. All calculations of theoretical m/z assume singly charged ions. All mentions of sialic acid (Sia) in this article refer to N-Acetylneuraminic acid (NeuAc).

FIG. 10. Provides a table showing theoretical glycopeptide masses for H1AH10 synthesized and glycosylated in vitro. Theoretical, doubly charged, monoisotopic mass-to-charge ratios (m/z) of the tryptic peptide containing the N-terminal, engineered glycosylation site within H1AH10 which was synthesized and glycosylated a one-pot in vitro reaction. Predicted glycosylation structures are based on previously established GT activities shown in FIGS. 2 and 3 and FIG. 8. Experimentally observed masses are annotated on deconvoluted MS and MS/MS spectra in FIGS. 4 and 25.

FIG. 11. Provides a table showing theoretical glycoprotein and glycopeptide masses for Fc-6 synthesized and glycosylated in the E. coli cytoplasm. Predicted glycosylation structures are based on previously established GT activities shown in FIGS. 2 and 3 and FIG. 8. Theoretical, neutral, average masses of expected glycoprotein products and theoretical, triply charged, monoisotopic mass-to-charge ratios (m/z) of glycopeptides are shown in the table. Glycopeptide masses correspond to the only ApNGT glycosylation site within Fc-6 which is contained within the tryptic peptide EEATTGGNWTTAGGR. Experimentally observed masses are annotated on deconvoluted MS and MS/MS spectra in FIGS. 4 and 26.

FIG. 12. Coomassie-stained protein gels showing CFPS expression of GlycoPRIME target and enzymes. Coomassie-stained protein gels of the soluble fractions of E. coli crude lysate based CFPS reactions following in vitro synthesis of Im7-6 target and indicated GlycoPRIME enzymes. Highly enriched proteins are evident from increased band thicknesses near expected molecular weights (arrows), other products can be seen in FIG. 13. Products from CFPS reactions run under oxidizing conditions indicated by (*). Soluble samples were isolated by centrifugation at 12,000× g for 15 min at 4° C. Representative of n=2 gels. The same gels were exposed as autoradiograms to determine bands containing [14C]-leucine protein (FIG. 13).

FIG. 13. Autoradiograms of protein gels showing CFPS expression of GlycoPRIME target and enzymes in CFPS. Autoradiograms of protein gels of the soluble fractions of E. coli crude lysate based CFPS reactions containing [14C]-leucine following in vitro synthesis of Im7-6 target and indicated GlycoPRIME enzymes. The presence of bands containing [14C]-leucine near expected molecular weights indicate full-length expression of proteins without large truncations (arrows indicate expected full-length product). Products from CFPS reactions run under oxidizing conditions indicated by (*). Soluble samples were isolated by centrifugation at 12,000× g for 15 min at 4° C. The autoradiograms were generated by exposing a 4-12% SDS-PAGE gel run in MOPS to a phosphoscreen for a 72-h. The autoradiogram is representative of n=2 gels and exposures. The same gels were Coomassie stained (Supplementary FIG. 1) and aligned with autoradiogram images for molecular weight standard reference.

FIG. 14. Glycopeptide MS/MS spectra of GlycoPRIME reaction products from two enzyme biosynthetic pathways elaborating N-linked glucose. Products from IVG reactions containing two enzyme pathways modifying Im7-6 shown in FIG. 2 were purified, trypsinized, and analyzed by pseudo Multiple Reaction Monitoring (MRM) MS/MS fragmentation at theoretical glycopeptide masses (red diamonds) corresponding to detected protein MS peaks using a collisional energy of 30 eV (see Methods). Spectra representative of many MS/MS acquisitions from n=1 IVG reaction. Theoretical protein, peptide, and sugar ion masses derived from expected glycosylation structures are shown in FIGS. 7 and 9. All indicated sugar ions are singly charged and glycopeptide fragmentation products are triply charged ions consistent with modification of Im7-6 tryptic peptide EATTGGNWTTAGGDVLDVLLEHFVK with indicated sugar structures. (a) MS/MS spectra of 999.49 ±2 m/z corresponding to N-linked Glcβ1-3GalNAc installed by BfGalNAcT. (b) MS/MS spectra of 1418.29 ±2 m/z corresponding to N-linked dextran polymer installed by Apα1-6. (c) MS/MS spectra of 985.81±2 m/z corresponding with N-linked lactose installed by NmLgtB. All IVG reactions contained Im7-6, ApNGT, and appropriate sugar donors according to established enzyme activities (FIG. 8).

FIG. 15. Deconvoluted intact protein MS spectra of IVG reaction products showing no modification of N-linked glucose installed by ApNGT. Products of IVG reactions containing 10 μM Im7-6, 0.4 μM ApNGT, 2.5 mM of appropriate sugar donors, and one elaborating GT were purified and analyzed by intact protein MS (see Methods). (a) Deconvoluted intact protein MS spectra of IVG containing 1.3 μM of Hpβ4GalT. (b) Deconvoluted intact protein MS spectra of IVG containing 1.4 μM of Btβ4GalT1 supplemented with 10 μM α-lactalbumin and performed under oxidizing conditions (see Methods). (c) Deconvoluted intact protein MS spectra of IVG containing 1.5 μM of SpWchJ and 1.0 μM of SpWchK. No peaks were detected that indicated the modification of Im7-6 with N-linked glucose installed by ApNGT (theoretical mass values shown in FIG. 7). Spectra from m/z 100-2000 were deconvoluted into 11,000-14,000 Da using Bruker Compass Data Analysis maximum entropy method. Deconvoluted spectra shown here are representative of n=2 IVG reactions.

FIG. 16. Optimization of LgtB homolog and concentration. Products of IVG reactions containing 10 μM Im7-6, 0.4 μM ApNGT, 2.5 mM of appropriate sugar donors, and indicated concentrations of NmLgtB or NgLgtB were purified and analyzed by intact protein MS (see Methods). (a) Deconvoluted intact protein MS spectra from IVG reactions containing indicated concentrations of NmLgtB. (b) Deconvoluted intact protein MS spectra from IVG reactions containing indicated concentrations of NgLgtB. Results representative of n=2 IVG reactions conducted for 24 h at 30° C. indicate that NmLgtB produced in CFPS has greater specific activity and that nearly homogeneous N-linked lactose can be obtained with 2 μM NmLgtB. Theoretical mass values shown in FIG. 7. All spectra were acquired from full elution peak areas of all detected glycosylated and aglycosylated Im7-6 species and were deconvoluted from m/z 100-2000 into 11,000-14,000 Da using Bruker Bruker Compass Data Analysis maximum entropy method.

FIG. 17. Optimization of sialyltranferase homologs. Deconvoluted intact protein MS spectra representative of n=2 IVG reactions containing 0.4 μM ApNGT, 2 μM NmLgtB, each sialyltranferase shown in FIG. 3, and 2.5 mM each of UDP-Glc, UDP-Gal, and CMP-Sia. Lysates enriched with sialyltransferases by CFPS were added with equal volumes to each IVG reaction such that each 32 μl-IVG reaction contained a total of 25 μl of CFPS lysates. These reactions contained 12.9 μM PpST3; 9.8 μM VsST3; 1.8 μM PmST3,6; 1.3 μM CjCST-I; 5.6 μM P1ST6; 0.7 μM of HsSIAT1; and 4.9 μM PdST6, based on CFPS yields shown in FIG. 6. CjCST-I and HsSIAT1 were synthesized in CFPS with oxidizing conditions because they were found to be more active when produced in this way (FIG. 20). Under the conditions above, the reaction containing PdST6 provided the most efficient conversion to 6′-siallylactose and the reaction containing CjCST-I provided the most efficient conversion to 3′-siallylactose (exoglycosidase digestions to confirm linkages are shown in FIG. 21). Although only trace amounts appear in PpST6 and VsST3, MS/MS detection and identification shows that these enzymes are functional (FIG. 18). All spectra were acquired from full elution peak areas of all detected glycosylated and aglycosylated Im7-6 species and were deconvoluted from m/z 100-2000 into 11,000-14,000 Da using Bruker Compass Data Analysis maximum entropy method.

FIG. 18. Glycopeptide MS/MS spectra of GlycoPRIME reaction products from three enzyme biosynthetic pathways elaborating N-linked lactose. Products from IVG reactions containing three enzyme pathways modifying Im7-6 shown in FIG. 3 were purified, trypsinized, and analyzed by pseudo MRM MS/MS fragmentation at theoretical glycopeptide masses (indicated by red diamonds) corresponding to detected protein MS peaks in FIG. 3 and FIG. 17. All glycopeptides were fragmented using a collisional energy of 30 eV with a window of ±2 m/z from targeted m/z values (see Methods). Spectra are representative of many MS/MS acquisitions from n=1 IVG reaction. Theoretical protein, peptide, and sugar ion masses derived from expected glycosylation structures are shown in FIGS. 7, and 9. All indicated sugar ions are singly charged and glycopeptide fragmentation products are triply charged ions consistent with modification of Im7-6 tryptic peptide EATTGGNWTTAGGDVLDVLLEHFVK with indicated sugar structures. Predicted sugar linkages based on previously established GT activities (FIG. 8) and exoglycosidase sequencing (FIGS. 21 and 22). All IVG reactions contained Im7-6, ApNGT, NmLgtB, indicated GTs, and appropriate sugar donors according to established GT activities.

FIG. 19. HdGlcNAcT does not modify the N-linked lactose substrate installed by ApNGT and NmLgtB. Deconvoluted intact protein MS spectra of IVG reaction product containing 10 μM Im7-6, 0.4 μM ApNGT, 2 μM NmLgtB, 1.5 μM HdGlcNAcT, and 2.5 mM of UDP-Glc, UDP-Gal, and UDP-GlcNAc. No peaks were detected that indicated the modification of Im7-6 with N-linked lactose installed by ApNGT and NmLgtB (see FIG. 7 for theoretical mass values). Deconvoluted spectra representative of n=2 IVG reactions.

FIG. 20. CjCST-I and HsSIAT1 exhibit greater activity when produced in oxidizing conditions. Deconvoluted intact protein MS spectra representative of of n=2 IVG reaction products containing 10 μM Im7-6, 0.4 μM ApNGT, 2 μM NmLgtB, 2.5 mM of UDP-Glc, UDP-Gal, and CMP-Sia as well as CjCST-I or HsSIAT1 made in CFPS conducted under oxidizing conditions, reducing conditions with supplemented the E. coli disulfide bond isomerase (DsbC), or standard reducing conditions (see Methods). CFPS conditions are known to create a protein synthesis environment conducive to disulfide bond formation as previously described24. Lysates enriched with sialyltranferases by CFPS were added in equal volumes. Therefore, reducing reaction conditions contained 1.9 μM of CjCST-I or 3.8 μM of HsSIAT1 while oxidizing reaction conditions reactions contained 1.3 μM of CjCST-I and 0.7 μM of HsSIAT1 (detailed CFPS yield information shown in FIG. 15). Aside from CFPS synthesis conditions for the CjCST-I and HsSIAT1, IVG reactions were performed identically without ensuring an oxidizing environment for glycosylation. Im7-6, ApNGT, and NmLgtB were produced with standard CFPS reaction conditions. Relative glycosylation efficiencies indicate that the oxidizing CFPS environment of CFPS allows for greater enzyme activities per unit of CFPS reaction volume and per μM of enzyme. This observation makes sense for HsSIAT1 which is normally active in the oxidizing environment of the human golgi and is known to contain disulfide bonds. Interestingly, an oxidizing synthesis environment also seems to benefit the activity of CjCST-I which does not contain disulfide bonds. However, the increased activity of CjCST-I cannot be explained by the general chaperone activity of DsbC.

FIG. 21. Exoglycosidase sequencing of Im7-6 modified by GlycoPRIME biosynthetic pathways containing sialic acids. Completed IVG reactions from the GlycoPRIME workflow where purified using Ni-NTA magnetic beads, incubated at 37° C. for at least 4 h with and without indicated commercially available exoglycosidases, trypsinized overnight, and then analyzed by glycopeptide LC-MS. The α2-3 Neuraminidase S was able to remove the sialic acids installed by CjCST-I; PmST3,6; and the first sialic acid installed by CjCST-II, indicating that these enzymes were installed sialic acids with α2-3 linkages. Sialic acids installed by PdST6, HsSIAT1, as well as the second and third sialic acids installed by CjCST-II were resistant to digestion by α2-3 Neuraminidase S but were susceptible to cleavage by an α2-3,6,8 Neuraminidase which is consistent with the established α2-6 activity of PdST6 and HsSIAT1 and the α2,8 linkages installed by CjCST-II in subsequent sialic acid additions. See Methods section for exoglycosidase details. All spectra were acquired from full elution peak areas of all detected glycosylated and aglycosylated species of the Im7-6 tryptic peptide EATTGGNWTTAGGDVLDVLLEHFVK containing an ApNGT glycosylation acceptor sequence. All indicated glycopeptide products are triply charged ions consistent with this Im7-6 tryptic peptide modified with indicated sugar structures.

FIG. 22. Exoglycosidase sequencing of Im7-6 modified by GlycoPRIME biosynthetic pathways not containing sialic acids. Completed IVG reactions from the GlycoPRIME workflow where purified using Ni-NTA magnetic beads, incubated at 37° C. for at least 4 h with and without indicated commercially available exoglycosidases, trypsinized overnight, and then analyzed by glycopeptide LC-MS. The sugars installed by NmLgtB, BtGGTA, HpFutA, and HpFutC were susceptible to cleavage by commercially available β1-4 Galactosidase S; α1-3,6 Galactosidase; α1-3,4 Fucosidase; and α1-2 Fucosidase, respectfully. The galactose installed by NmLgtC was resistant to cleavage by β1-4 Galactosidase S and α1-3,6 Galactosidase, but susceptible to cleavage by α1-3,4,6 Galactosidase. The LacNAc polymer installed by alternating activities by NmLgtB and NgLgtA was susceptible to cleavage by a mixture of β1-4 Galactosidase S and the β-N-Acetylglucosaminidase S. All spectra were acquired from full elution peak areas of all detected glycosylated and aglycosylated species of the Im7-6 tryptic peptide EATTGGNWTTAGGDVLDVLLEHFVK containing an ApNGT glycosylation acceptor sequence. All indicated glycopeptide products are triply charged ions consistent with this Im7-6 tryptic peptide modified with indicated sugar structures. Cleavage observations are consistent with previously established GT activities (FIGS. 2-3, and 8). See Methods section for exoglycosidase details.

FIG. 23. Glycopeptide MS/MS spectra of GlycoPRIME reaction products from four and five enzyme biosynthetic pathways elaborating N-linked lactose. Products from IVG reactions containing four and five enzyme pathways modifying Im7-6 shown in FIG. 3d and FIG. 25 were purified, trypsinized, and analyzed by pseudo MRM MS/MS fragmentation at theoretical glycopeptide masses (indicated by red diamonds) corresponding to detected protein MS peaks in FIG. 3d and FIG. 25. All glycopeptides were fragmented using a collisional energy of 30 eV with a window of ±2 m/z from targeted m/z values (see Methods). Spectra representative of many MS/MS acquisitions from n=1 IVG reaction. Theoretical protein, peptide, and sugar ion masses derived from expected glycosylation structures are shown in FIGS. 7 and 9. All indicated sugar ions are singly charged and glycopeptide fragmentation products are triply charged ions consistent with modification of Im7-6 tryptic peptide EATTGGNWTTAGGDVLDVLLEHFVK with indicated sugar structures. Predicted sugar linkages based on previously established GT activities (FIG. 8). Although products from five-enzyme biosynthetic pathway product could not be unambiguous defined, sugar and glycopeptide fragments do suggest modification with both fucose and sialic acids. All IVG reactions contained Im7-6, ApNGT, NmLgtB, indicated enzymes, and appropriate sugar donors according to established GT activities.

FIG. 24. Deconvoluted intact protein MS spectra of IVG reaction products showing no production fucosylated and sialylated species. Products of IVG reactions containing 10 μM Im7-6, 0.4 μM ApNGT, 2 μM NmLgtB, indicated enzymes, and 2.5 mM of appropriate sugar donors (UDP-Glc, UDP-Gal, CMP-Sia, and GDP-Fuc) were purified and analyzed by intact protein MS. Reactions contained 2.4 μM HpFutA and 2.4 μM PdST6 or 1.3 μM HpFutC and 0.65 μM CjCST-I as indicated. Deconvoluted spectra representative of n=2 IVGs. No peaks were detected that indicated the presence of Im7-6 modified with both a sialic acid and a fucose (the region of the spectra annotated by arrows [between 12000 and 12200] shows expected range of sialylated and fucosylated species) (see FIG. 8 for theoretical mass values).

FIG. 25. GlycoPRIME screening of biosynthetic pathways containing five enzymes. Products of IVG reactions containing 10 μM Im7-6, 0.4 μM ApNGT, 2 μM NmLgtB, indicated GTs, and 2.5 mM of appropriate sugar donors (UDP-Glc, UDP-Gal, CMP-Sia, and GDP-Fuc) were purified from and analyzed by intact protein MS. Deconvoluted spectra representative of n=2 IVGs. (a) Deconvoluted intact protein MS of IVG reactions containing 0.87 μM HpFutC, 3.83 μM NgLgtA, and 1.63 μM PdST6. (b) Deconvoluted intact protein MS of IVG reactions containing 1.63 μM HpFutA, 3.83 μM NgLgtA, and 1.63 μM PdST6 (also shown in FIG. 3d) (c) Deconvoluted intact protein MS of IVG reactions containing 1.63 μM HpFutA, 3.83 μM NgLgtA, and 0.43 μM CjCST-I. (d) Deconvoluted intact protein MS of IVG reactions containing 0.87 μM HpFutC, 3.83 μM NgLgtA, and 0.43 μM CjCST-I. Spectra in a and b as well as fragmentation spectra in FIG. 23 indicated three and one species, respectively, which contained both sialic acid and fucose. Predicted glycosylation structures based on previously established GT activities (FIG. 8) and fragmentation spectra (FIG. 23). Although structures cannot be unambiguously identified, the previously observed incompatibility of HpFutA and PdST6 as well as the presence of a 1083 m/z peak (Glcβ4Galα6Sia) and the absence of a 1034 m/z (Glc(α3Fuc)β4Gal) peak in fragmentation spectra suggests that in b the proximal galactose is modified with a sialic acid while the GlcNAc is modified with the fucose. No peaks in c or d were detected that indicated the presence of Im7-6 modified with both a sialic acid and a fucose (see FIG. 7 for theoretical mass values).

FIG. 26. Intact protein MS spectra of Im7-6 synthesized and glycosylated by CFPS-GpS reactions. (a) Plasmids encoding the Im7-6 target protein and sets of up to three GTs based on 12 successful biosynthetic pathways developed by two-pot GlycoPRIME screening were combined with appropriate sugar donors in one-pot CFPS-GpS reactions and incubated for 24 h at 30° C. (b) Deconvoluted intact protein spectra from Im7-6 synthesized and glycosylated in CFPS-GpS reactions with and without ApNGT plasmid. (c) Deconvoluted intact protein spectra from Im7-6 synthesized and glycosylated in CFPS-GpS reactions with ApNGT plasmid and indicated GT plasmids. (d) Deconvoluted intact protein spectra from Im7-6 synthesized and glycosylated in CFPS-GpS reactions with ApNGT, NmLgtB, and indicated GT plasmids. All reactions contained equimolar amounts of each plasmid and a total plasmid concentration of 10 nM. All Im7-6 proteins were purified using Ni-NTA magnetic beads before intact protein analysis (see Methods). All reactions showed intact protein mass shifts consistent with the modification of Im7-6 with the same glycans observed in our two-pot system (FIGS. 2-3), although at lower efficiency. MS spectra were acquired from full elution areas of all detected glycosylated and aglycosylated protein or peptide species and are representative of n=2 CFPS-GpS reactions. Deconvoluted spectra collected from m/z 100-2000 into 11,000-14,000 Da using Bruker Compass Data Analysis maximum entropy method. See FIG. 16 for theoretical mass values.

FIG. 27. Production of sialylated Im7-6 in the E. coli cytoplasm. (a) Design of cytoplasmic glycosylation system to produce sialylated glycoproteins in E. coli. Three plasmids containing NmNeuA (CMP-Sia synthesis), target protein containing ApNGT glycosylation acceptor sequence, and biosynthetic pathways discovered using GlycoPRIME (GT operon). (b-f) Deconvoluted intact protein spectra from Im7-6 purified from CLM24ΔnanA E. coli strain containing CMP-Sia synthesis plasmid and Im7-6 target protein plasmid as well as no GT operon b; GT operon containing ApNGT c; GT operon containing ApNGT and LgtB d; GT operon containing ApNGT, NmLgtB, and CjCST-I e; or GT operon containing ApNGT, NmLgtB, and PdST6 f. The last GT in all glycosylation pathways is indicated. Mass shifts in intact protein spectra are consistent with established activities of each GT and the installation of N-linked Glc, lactose, 3′-sialyllactose, and 6′-sialyllactose onto Im7-6 in b, c, d, e, and f, respectively. All E. coli cultures were supplemented with 5 mM sialic acid and grown to OD600=0.6 at 37° C., induced with 1 mM IPTG and 0.2% arabinose, and then incubated overnight at 25° C. MS spectra were acquired from full elution areas of all detected glycosylated and aglycosylated protein species and were deconvoluted from m/z 100-2000 into 11,000-14,000 Da using Bruker Compass Data Analysis maximum entropy method. See FIG. 7 for theoretical masses. Spectra representative of n=2 bacterial cultures.

FIG. 28. Exoglycosidase sequencing of Fc glycosylated in the E. coli cytoplasm. (a) Deconvoluted intact protein spectra from Fc-6 purified from CLM24ΔnanA E. coli strain containing CMP-Sia synthesis plasmid, Fc-6 target protein plasmid, and a GT operon plasmid containing ApNGT, NmLgtB, and PdST6. (b-d) Purified Fc-6 from a was incubated at 37° C. for at least 4 h with commercially available α2-3 Neuraminidase S b, α2-3,6,8 Neuraminidase c, or β1-4 Galactosidase S and α2-3,6,8 Neuraminidase d. Resistance of terminal sialic acid to α2-3 Neuraminidase S and susceptibility to α2-3,6,8 Neuraminidase indicates an α2-6 linkage, which is consistent with previously established activity of PdST6 (FIG. 8). (e) Deconvoluted intact protein spectra from Fc-6 purified from CLM24ΔnanA E. coli strain containing CMP-Sia synthesis plasmid, Fc-6 target protein plasmid, and a GT operon plasmid containing ApNGT, NmLgtB, and CjCST-I. (f-g) Purified Fc-6 from e was incubated at 37° C. for at least 4 h with commercially available α2-3 Neuraminidase S b, or β1-4 Galactosidase S and α2-3 Neuraminidase S. Susceptibility of terminal sialic acid to α2-3 Neuraminidase confirms the previously established activity of CjCST-I (FIG. 8). Removal of middle galactose with addition β1-4 Galactosidase S in d and g confirms the previously established activity of NmLgtB (FIG. 8). a-c and e-f are also shown in FIG. 4. See Methods for exoglycosidase details and FIG. 11 for theoretical glycoprotein masses. All E. coli cultures were supplemented with 5 mM sialic acid and grown to OD₆₀₀ =0.6 at 37° C. then induced with 1 mM IPTG and 0.2% arabinose then incubated overnight at 25° C. MS spectra were acquired from full elution areas of all detected glycosylated and aglycosylated protein species and were deconvoluted from m/z 100-2000 into 27,000-29,000 Da using Bruker Compass Data Analysis maximum entropy method.

FIG. 29. Shows the DNA sequences encoding engineered glycosylation targets, in vitro expressed glycosyltransferases, in vivo glycosyltransferases operons, and in vivo CMP-Sia production plasmid. Key: TRANSLATED REGION; Engineered glycosylation acceptor sequence; FLANKING REGIONS ADJACENT TO GLYCOSYLATION ACEPTOR SEQUENCES; [symbol] terminator;

[symbol]

FIG. 30. Is a schematic showing glycosylation using non-standard sugars in living E. coli.

FIG. 31. Deconvoluted glycoprotein MS results, showing successful modification of model protein Im7 (with ATTCCNWTTAGG grafted into an exposed loop) with Azido-sialic acid with α2,3, and α2, 6 linkages.

FIG. 32. Deconvoluted glycoprotein MS results, showing successful modification of model protein human Fc (with ATTGGNWTTAGG replacing the natural QYNSTY glycosylation site on Fc) with Azido-sialic acid with α2,3, and α2, 6 linkages.

FIG. 33. Provides a schematic showing site-directed glycoPEGylation of an exemplary therapeutic compound, and exemplary “click”-able siglec-binding ligands for tolerogenic responses.

### DETAILED DESCRIPTION

### Introduction

Glycosylation endows protein therapeutics with beneficial properties including increased serum half-life and the ability to elicit protective immune responses. Developments in genetic editing, engineered microbial strains, and in vitro synthesis systems promise new opportunities for glycoprotein therapeutics. However, constructing biosynthetic pathways to engineer protein glycosylation remains a key bottleneck. Here, the inventors developed and employed a modular cell-free platform for glycosylation pathway assembly by rapid in vitro mixing and expression (GlycoPRIME). In GlycoPRIME, crude Escherichia coli lysates are enriched with glycosyltransferases by cell-free protein synthesis and then glycosylation pathways are assembled to elaborate a single glucose priming handle installed by a soluble, N-linked glycosyltransferase. The inventors used GlycoPRIME to construct 37 putative protein glycosylation pathways, creating 23 unique glycan motifs. Many of these pathways have not been previously described and produce glycosylation structures of interest for protein therapeutics and vaccines. The inventors then used selected biosynthetic pathways to produce glycoproteins the constant region of a human antibody with minimal sialic acid glycans in living E. coli and a protein vaccine candidate with adjuvanting glycans in on-demand a cell-free expression platform. GlycoPRIME and the pathways described here could accelerate the engineering of glycoproteins with defined properties and the manufacturing of glycoproteins in alternative hosts.

### Definitions and Terminology

The disclosed components, systems, and methods for glycoprotein and recombinant glycoprotein protein synthesis may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “an oligosaccharide” or “a glycosyltransferase” should be interpreted to mean “one or more oligosaccharides” and “one or more glycosyltransferase,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of ” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 'B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors which serve equivalent functions.

In certain exemplary embodiments, the recombinant expression vectors comprise a nucleic acid sequence in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

Oligonucleotides and polynucleotides may optionally include one or more non-standard nucleotide(s), nucleotide analog(s) and/or modified nucleotides. Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-i sopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 28ufa28hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2nd edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length>100 amino acids (Garrett & Grisham, Biochemistry, 2nd edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide or polypeptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein), glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Modified amino acid sequences that are disclosed herein may include a deletion in one or more amino acids. As utilized herein, a “deletion” means the removal of one or more amino acids relative to the native amino acid sequence. The modified amino acid sequences that are disclosed herein may include an insertion of one or more amino acids. As utilized herein, an “insertion” means the addition of one or more amino acids to a native amino acid sequence. The modified amino acid sequences that are disclosed herein may include a substitution of one or more amino acids. As utilized herein, a “substitution” means replacement of an amino acid of a native amino acid sequence with an amino acid that is not native to the amino acid sequence. For example, the modified amino sequences disclosed herein may include one or more deletions, insertions, and/or substitutions in order modified the native amino acid sequence of a target protein to include one or more heterologous amino acid motifs that are glycosylated by an N-glycosyltransferase.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The peptides, polypeptides, and proteins contained herein may include or may be modified to include an amino acid receptor motif for a glycosyltransferase. For example, the peptides, polypeptides, and proteins contained herein may include or may be modified to include an amino acid receptor motif comprising N-X-S/T, which is an amino acid receptor motif for N-linked glycosyltransferases (NGTs) as discussed herein (e.g., ApNGT).

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | Conservative |
|---|---|
| Residue | Substitution |
| Ala | Gly, Ser |
| Arg | His, Lys |
| Asn | Asp, Glu, His |
| Asp | Asn, Glu |
| Cys | Ala, Ser |
| Gln | Asn, Glu, His |
| Glu | Asp, Glu, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

### Cell-Free Protein Synthesis (CFPS)

The components, systems, and methods disclosed herein may be applied to cell-free protein synthesis methods as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014-0295492, 2014-0255987, 2014-0045267, 2012-0171720, 2008-0138857, 2007-0154983, 2005-0054044, and 2004-0209321. See also U.S Published Application Nos. 2005-0170452; 2006-0211085; 2006-0234345; 2006-0252672; 2006-0257399; 2006-0286637; 2007-0026485; 2007-0178551. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non standard amino acids,” U.S. Patent Application Ser. No.: 62/044,221; Jewett, M. C., Hodgman, C. E., and Gan, R. 2013, “Methods for yeast cell-free protein synthesis,” U.S. Patent Application Ser. No.: 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No.: 62/098,578. See also Guarino, C., & DeLisa, M. P. (2012). A prokaryote-based cell-free translation system that efficiently synthesizes glycoproteins. Glycobiology, 22(5), 596-601. The contents of all of these references are incorporated in the present application by reference in their entireties.

In some embodiments, a “CFPS reaction mixture” typically may contain one or more of a crude or partially-purified cell extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

As used herein, “translation template” for a polypeptide refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

A reaction mixture may include an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the reaction mixture may comprise a coupled transcription/translation (“Tx/T1”) system where synthesis of translation template and a sequence defined biopolymer from the same cellular extract.

The reaction mixture may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts (for examples, S12, S30 and S60 extracts).

The temperature may be any temperature suitable for CFPS. Temperature may be in the general range from about 10° C. to about 40° C., including intermediate specific ranges within this general range, include from about 15° C. to about 35° C., from about 15° C. to about 30° C., from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The reaction mixture may include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The reaction mixture may include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The reaction mixture may include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The reaction mixture may include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The reaction mixture may include endogenous NTPs (i.e., NTPs that are present in the cell extract) and or exogenous NTPs (i.e., NTPs that are added to the reaction mixture). In certain aspects, the reaction use ATP, GTP, CTP, and UTP. In certain aspects, the concentration of individual NTPs is within the range from about 0.1 mM to about 2 mM.

The reaction mixture may include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, translation, and/or glycosylation steps) described herein. Examples of vessels include, but are not limited to, a microtitre plate, a test tube, a microfuge tube, a beaker, a flask, a multi-well plate, a cuvette, a flow system, a microfiber, a microscope slide and the like.

### Glycosylation of Proteins

The components, systems, and methods disclosed herein may be applied to recombinant cell systems and cell-free protein synthesis methods in order to prepare glycosylated proteins. Glycosylated proteins that may be prepared using the disclosed components, systems, and methods may include proteins having N-linked glycosylation (i.e., glycans attached to nitrogen of asparagine). The glycosylated proteins disclosed herein may include unbranched and/or branched sugar chains composed of monosaccharides as known in the art such as glucose (e.g., β-D-glucose), galactose (e.g., β-D-galactose), mannose (e.g., β-D-mannose), fucose (e.g., α-L-fucose), N-acetyl-glucosamine (GlcNAc), N-acetyl-galactosamine (GalNAc), N-acetyl-glucosamine, pyruvic acid, neuraminic acid, N-acetylneuraminic acid (i.e., sialic acid), and xylose, which may be attached to the glycosylated proteins, growing glycan chain, or donor molecule (e.g., a sugar donor nucleotide) via respective glycosyltransferases. Other monosaccharides for glycosylating proteins may include allose, altrose, gulose, idose, talose, ribose, arabinose, lyxose. Other monosaccharides for glycosylating proteins may include deoxy monosaccharides such as deoxyribose. In addition, non-natural sugars are also useful for glycosylating proteins due to their unique biophysical properties (including surface charge and hydrogen bonding), unique binding profiles to endogeneous receptors (including lectins and siglecs), potential for further modification by biorthogonal or semi-bioorthogonal conjugation methods (including click chemistry and Michael addition), and differences in their ability to be physically degraded or enzymatically degraded or removed (including by glycosidases). These non-natural sugars include but are not limited to sugars with azido, alkyne, or strained alkynes/alkene functional groups sugars (including azido-sialic acid, (azido-Sia)); sugars with thiol or maleimide groups; deoxysugars; PEGylated sugars; amino sugars; pre-assembled oligo- or polysaccharides containing natural and/or non-natural monomers; fluorinated sugars; and others.

### Glycosylation in Prokaryotes

Glycosylation in prokaryotes is known in the art. (See e.g., U.S. Pat. Nos. 8,703,471; and 8,999,668; and U.S. Published Application Nos. 2005/0170452; 2006/0211085; 2006/0234345; 2006/0252672; 2006/0257399; 2006/0286637; 2007/0026485; 2007/0178551; and International Published Applications WO2003/056914A1; WO2004/035605A2; WO2006/102652A2; WO2006/119987A2; and WO2007/120932A2; the contents of which are incorporated herein by reference in their entireties).

### Modular Platform for Producing Glycoproteins and Identifying Glycosylation Pathways

The inventors have disclosed components, systems, and methods for glycoprotein protein synthesis in vitro and in vivo. In particular, the inventors have disclosed components, systems, and methods that relate to modular platforms for producing glycoproteins. The components, systems, and methods disclosed by the inventors may be used in synthesizing glycoproteins and recombinant glycoproteins in cell-free protein synthesis (CFPS) and in modified cells.

In one embodiment, the inventors have disclosed a cell-free system for glycosylating a peptide or polypeptide sequence in vitro. The peptide or polypeptide sequence may be present in a peptide (i.e., a relatively short amino acid sequence) or a polypeptide (i.e., a relatively longer amino acid sequence), the peptide or polypeptide sequence typically comprises an asparagine residue which can be glycosylated by an N-linked glycosyltransferase. For example, the peptide or polypeptide sequence may comprise the amino acid motif N-X-S/T. The disclosed systems may comprise as components: (i) a glycosyltransferase which is a soluble N-linked glycosyltransferase (as used herein the terms “N-linked glycosyltransferase” and “N-glycosyltransferase” and “NGT” are used interchangably) that catalyzes transfer to an amino group of the asparagine residue a monosaccharide (optionally where the monosaccharide is glucose (Glc)) to provide an N-linked glycan, or an expression vector that expresses the NGT in a cell-free protein synthesis (CFPS) reaction mixture; (ii) a glycosylation mixture comprising a monosaccharide donor (optionally a Glc donor; optionally, a monosaccharide; as used herein, the term “monosaccharide donor” includes, but is not limited to a monosaccharides and polysaccharides); where the peptide or polypeptide sequence is glycosylated in the glycosylation mixture in vitro to provide a peptide or polypeptide sequence comprising the N-linked glycan (optionally an N-linked Glc). In some embodiments, the NGT is membrane bound.

In further embodiments of the disclosed systems, the systems further may comprise as a component: (iii) a second glycosyltransferase that is soluble and catalyzes transfer to the N-linked glycan a monosaccharide (optionally where the monosaccharide is Glc, galactose (Gal), N-acetylgalactosamine (GalNAc), N-acetylglucosamine (GlcNAc), pyruvate, fucose (Fuc), sialic acid (Sia)), or an expression vector that expresses the second glycosyltransferase in a cell-free protein synthesis (CFPS) reaction mixture; where the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan is glycosylated with one or more moieties selected from Glc, Gal, GalNAc, GlcNAc, pyruvate, Fuc, and Sia (optionally to provide N-linked dextrose, N-linked lactose, or N-linked Glc-GalNAc). In some embodiments, the second glycosyltransferase is membrane bound.

In even further embodiments of the disclosed systems, the systems further may comprise as a component: (iv) a third glycosyltransferase that is soluble and that catalyzes transfer to the N-linked glycan a monosaccharide (optionally where the monosaccharide is Glc, Gal, GalNAc, GlcNAc, pyruvate, Fuc, Sia, or combinations thereof), or an expression vector that expresses the third glycosyltransferase in a cell-free protein synthesis (CFPS) reaction mixture; where the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan further is glycosylated with one or more moieties selected from Glc, Gal, GalNAc, GlcNAc, pyruvate, Fuc, and Sia (optionally to provide an N-linked glycan comprising one or more moieties selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3Gal or GlcNAcβ1-4Galα1-3Gal)). As used herein, LacNAc is used interchangeably with Lactose-(poly)LacNAc. In some embodiments, the third glycosyltransferase is membrane bound.

The disclosed systems may include or utilize cell-free protein synthesis (CFPS) and/or components for performing CFPS. In some embodiments of the disclosed systems, the systems comprise or utilize a cell-free protein synthesis (CFPS) reaction mixture and one or more of the first glycosyltransferase, the second glycosyltransferase, and the third glycosyltransferase are present or expressed in the CFPS reaction mixture. In further embodiments of the disclosed systems, the systems comprise or utilize one or more cell-free protein synthesis (CFPS) reaction mixtures and one or more of the first glycosyltransferase, the second glycosyltransferase, and the third glycosyltransferase are present or expressed in the CFPS reaction mixtures. Optionally, the one or more CFPS reaction mixtures may be combined to provide the disclosed systems and/or components for the disclosed systems. In some embodiments, the one or more CFPS reaction mixtures may be combined to create glycosylation pathways.

The disclosed systems may be utilized for glycosylating a peptide or polypeptide sequence. In some embodiments of the disclosed systems, the systems comprise the peptide or polypeptide sequence, or an expression vector that expresses the peptide or polypeptide sequence. Optionally, the peptide or polypeptide sequence may be provided and/or expressed in a cell-free protein synthesis (CFPS) reaction mixture.

Suitable CFPS reaction mixtures may comprise one or more components obtained from prokaryotic cells. For example, components for the CFPS reaction miztures may include prokaryotic cell lysates. Optionally, the cell lysates may be enriched in one or more glycosyltransferases as disclosed herein. In some embodiments, the CFPS reaction mixture may comprise or utilize a lysate prepared from Escherichia coli, optionally wherein the E. coli has been modified to express one or more components of the disclosed systems such as the glycosyltransferases disclosed herein.

The disclosed systems typically include and/or utilize a first glycosyltransferase. Optionally, the first glycosyltransferase may be a bacterial N-linked glycosyltransferase (NGT) or a modified NGT having one or more mutations relative to a wild-type NGT. Optionally, the bacterial NGT is a bacterial NGT selected from the group consisting of Actinobacillus pleuropneumoniae (ApNGT) (SEQ ID NO:1), Escherichia coli NGT (EcNGT) (SEQ ID NO:3), Haemophilus influenza NGT (HiNGT) (SEQ ID NO:5), Mannheimia haemolytica NGT (MhNGT) (SEQ ID NO:7), Haemophilus dureyi NGT (HdNGT) (SEQ ID NO:9), Bibersteinia trehalosi NGT (BtNGT) (SEQ ID NO:11), Aggregatibacter aphrophilus NGT (AaNGT) (SEQ ID NO:13), Yersinia enterocolitica (YeNGT) NGT (SEQ ID NO:15), Yersinia pestis (YpNGT) NGT (SEQ ID NO:17), and Kingella kingae (KkNGT) NGT (SEQ ID NO:19). In some embodiments, the NGT is soluble. In some embodiments, the NGT is membrane bound. Additional NGTs useful in the present compositions and methods can be found in PCT/US2018/000185, for example, Actinobacillus pleuropneumoniae (ApNGT) glycosyltransferase (NGT) having mutation Q469A.

In some embodiments, the disclosed systems may include and/or may express a glycosyltransferase for use in the disclosed methods such as a modified bacterial NGT comprising one or more mutations, for example, mutations that change peptide acceptor specificity and/or increase enzymatic turnover rates. (See Song et al., “Production of homogeneous glycoprotein with multisite modifications by an engineered N-glycosyltransferase mutant,” J. Biol. Chem., Apr. 5, 2017, 292, 8856-8863, the content of which is incorporated herein by reference in its entirety). In some embodiments, the modified bacterial NGT is a modified ApNGT having a substitution at Q469 for example where Q469 is replaced with an amino acid X, where X is selected from S, T, N, C, G, P, A, I, L, M, V (see, e.g., SEQ ID NO:2 having Q469A). In some embodiments, the modified bacterial NGT is a modified EcNGT having a substitution at F482 where F482 is replaced with an amino acid X, where X is selected from S, T, N, C, G, P, A, I, L, M, V (see, e.g., SEQ ID NO:4, having F482A). In some embodiments, the modified bacterial NGT is a modified HiNGT having a substitution at Q495 where Q495 is replaced with an amino acid X, where X is selected from S, T, N, C, G, P, A, I, L, M, V (see, e.g, SEQ ID NO:6 having Q495A). In some embodiments, the modified bacterial NGT is a modified MhNGT having a substitution at Q469 where Q469 is replaced with an amino acid X, where X is selected from S, T, N, C, G, P, A, I, L, M, V (see, e.g, SEQ ID NO:8 having Q469A). In some embodiments, the modified bacterial NGT is a modified HdNGT having a substitution at Q468 where Q468 is replaced with an amino acid X, where X is selected from S, T, N, C, G, P, A, I, L, M, V (see, e.g, SEQ ID NO:10 having Q468A). In some embodiments, the modified bacterial NGT is a modified BtNGT having a substitution at Q471 where Q471 is replaced with an amino acid X, where X is selected from S, T, N, C, G, P, A, I, L, M, V (see, e.g, SEQ ID NO:12 having Q471A). In some embodiments, the modified bacterial NGT is a modified AaNGT having a substitution at Q468 where Q468 is replaced with an amino acid X, where X is selected from S, T, N, C, G, P, A, I, L, M, V (see, e.g, SEQ ID NO:14 having Q468A). In some embodiments, the modified bacterial NGT is a modified YeNGT having a substitution at F466 where F466 is replaced with an amino acid X, where X is selected from S, T, N, C, G, P, A, I, L, M, V (see, e.g, SEQ ID NO:16 having F466A). In some embodiments, the modified bacterial NGT is a modified YpNGT having a substitution at F466 where F466 is replaced with an amino acid X, where X is selected from S, T, N, C, G, P, A, I, L, M, V (see, e.g, SEQ ID NO:18 having F466A). In some embodiments, the modified bacterial NGT is a modified KkNGT having a substitution at Q474 where Q474 is replaced with an amino acid X, where X is selected from S, T, N, C, G, P, A, I, L, M, V (see, e.g, SEQ ID NO:20 having Q474A).

In some embodiments, the disclosed systems may include and/or may express a glycosyltransferase having the amino acid sequence of any of SEQ ID NOs:1, 3, 5, 7, 9, 11, 13, 15, 17, or 19 or having a least 50%, 60%, 70%, 80%, 90%, 95%, 96%, 97%, 98%, or 99% sequence identity to any of SEQ ID NOs:1, 3, 5, 7, 9, 11, 13, 15, 17, or 19, or the first glycosyltransferase is a modified bacterial N-linked glycosyltransferase (NGT) having the amino acid sequence of any of SEQ ID NOs:2, 4, 6, 8, 10, 12, 14, 16, 18, or 20, or having a least 50%, 60%, 70%, 80%, 90%, 95%, 96%, 97%, 98%, or 99% sequence identity to any of SEQ ID NOs:2, 4, 6, 8, 10, 12, 14, 16, 18, or 20.

The disclosed systems may include and/or utilize a second glycosyltransferase. Optionally, the second glycosyltransferase is a bacterial glycosyltransferase. Optionally, the second glycosyltransferases is an α1-6 glucosyltransferase, a β1-4 galactosyltransferase, or a β1-3 N-acetylgalactosamine transferase. Optionally, the second glycosyltransferase is selected from the group consisting of Actinobacillus pleuropneumoniae α1-6 glucosyltransferase (Apα1-6), Neisseria gonorrhoeae β1-4 galactosyltransferase LgtB (NgLGtB), Neisseria meningitidis β1-4 galactosyltransferase LgtB (NmLGtB), and Bacteriodes fragilis β1-3 N-acetylgalactosamine transferase (BfGalNAcT).

The disclosed systems may include and/or utilize a third glycosyltransferase. Optionally, the third glycosyltransferase is a bacterial glycosyltransferase. Optionally, the third glycosyltransferases is a β1-3 N-acetylglucosamine transferase, a pyruvyltransferase, an α1-3 fucosyltransferase, an α1-2 fucosyltransferase, an α1-4 galactosyltransferase, an α1-3 galactosyltransferase, an α2-6 sialyltransferase, an α2-3,6 sialyltransferase, an α2-3 sialyltransferase, or an α2-3,8 sialyltransferase. Optionally, the third glycosyltransferase is selected from the group consisting of Neisseria gonorrhoeae β1-3 N-acetylglucosamine transferase (NgLgtA), Schizosaccharomyces pombe pyruvyltransferase (SpPvg1), Helicobacter pylori α1-3 fucosyltransferase (HpFutA), Helicobacter pylori α1-2 fucosyltransferase (HpFutC), Neisseria meningitidis α1-4 galactosyltransferase (NmLgtC), Bos taurus α1-3 galactosyltransferase (BtGGTA), Homo sapiens α2-6 sialyltransferase (HsSIAT1), Photobacterium damselae α2-6 sialyltransferase (PdST6), Photobacterium leiognathid α2-6 sialyltransferase (P1ST6), Pasteurella multocida α2-3,6 sialyltransferase (PmST3,6), Vibrio sp JT-FAJ-16 α2-3 sialyltransferase (VsST3), Photobacterium phosphoreum α2-3 sialyltransferase (PpST3), Campylobacter jejuni α2-3 sialyltransferase (CjCST-I), and Campylobacter jejuni α2-3,8 sialyltransferase (CjCST-II).

One or more of the components of the disclosed systems may be in a preserved form. In some embodiments, one or more components of the disclosed systems are freeze-dried.

Also disclosed are peptide or polypeptide sequences that comprise an N-linked glycan. Optionally, the disclosed peptide or polypeptide sequences are prepare using any of the systems disclosed herein or using any of the components of the systems disclosed herein. In some embodiments, the peptide or polypeptide sequence comprising an N-linked glycan where the N-linked glycan comprises a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3Gal or GlcNAcβ1-4Galα1-3Gal). In some embodiments, peptides or polypeptides including forms of lactose or lactose-(poly)LacNAc with one or more additions of fucose in α1,2 or α1,3 linkages and/or sialic acid in linkages of α2,3 or α2,6 are disclosed. In some embodiments, the disclosed peptides or polypeptides may be utilized or formulated for use as a therapeutic protein or a vaccine. As used herein, the term LacNAc is used interchangeably with Lactose-(poly)LacNAc.

Also disclosed herein are modified cells. The disclosed modified bacterial cells may include modified bacterial cells such as genetically modified bacterial cells. Genetically modified bacterial cells may include cells in which the genome of the cells has been modified to express a heterologous protein (e.g., a heterologous glycosyltransferase or peptide or polypeptide sequence for glycosylation) and cells that have been transformed by a epigenetic vector that expresses a heterologous protein (e.g., a heterologous glycosyltransferase or peptide or polypeptide sequence for glycosylation). The disclosed modified cells may comprise and/or express one or more of the components of the systems disclosed herein. The disclosed modified cells may be utilized to prepare one or more of the components of the systems disclosed herein. The disclosed modified cells may overexpress particular proteins or may be deficient in the expression of particular paroteins. By way of example, but not by way of limitation, in some embodiments, modified cells or cell lysates may be deficient in NanA (sialic acid aldolase), produced reduced amounts of NanA (sialic acid aldolase), or express nonfunctional or reduced function NanA (sialic acid aldolase).

In some embodiments, the modified cells and/or components of the modified cells may be utilized in methods disclosed herein for glycosylating a peptide or polypeptide sequence. In some embodiments of the disclosed methods for preparing a glycosylated peptide or polypeptide sequence in vivo, the methods comprising culturing a modified bacterial cell, wherein the modified bacterial cell comprises or expresses a peptide or polypeptide sequence for glycosylation, an N-linked glycosyltransferase, and/or one or more additional glycosyltransferases, and the peptide or polypeptide sequence is glycosylated in the modified bacterial cell or in a glycosylation reaction mixture. In some embodiments, in vivo glycosylation comprises a non-natural sugar (e.g., azido-modified sugars, including azido-sialic acids).

In some embodiments, components of the modified cells may be utilized in cell-free protein synthesis CFPS methods and/or glycosylation reaction methods. Components prepared from the modified cells may include, but are not limited to cell lysates, optionally wherein the lysates are suitable for use in CFPS reaction methods and/or glycosylation reaction methods, either alone or in combination with cell lysates prepared from other modified cells.

Also disclosed herein are methods for preparing a glycosylated peptide or polypeptide sequence in vitro. The methods may include reacting a peptide or polypeptide sequence comprising an asparagine residue (e.g., a peptide or polypeptide sequence comprising the amino acid motif N-X-S/T) in a glycosylation mixture comprising a monosaccharide donor (optionally wherein the monosaccharide donor is a glucose (Glc) donor, or wherein the monosaccharide donor is a monosaccharide) with a glycosyltransferase which is a soluble N-linked glycosyltransferase (as used herein the terms “N-linked glycosyltransferase,” “N-glycosyltransferase” and “NGT” are used interchangably) that catalyzes transfer of the monosaccharide from the monosaccharide donor (optionally Glc from the Glc donor or wherein the monosaccharide donor is a monosaccharide) to an amino group of the asparagine residue to provide an N-linked glycan (optionally an N-linked Glc). In the disclosed methods, the peptide or polypeptide sequence is glycosylated in the glycosylation mixture in vitro to provide a peptide or polypeptide sequence comprising the N-linked glycan (optionally an N-linked Glc). Optionally in the disclosed in vitro methods, the peptide or polypeptide sequence, the NGT, or both may be expressed in one or more cell-free protein synthesis (CFPS) reaction mixtures prior to performing the glycosylation reaction. Optionally, the peptide or polypeptide sequence may be expressed in a first CFPS reaction mixture, and/or the NGT may be expressed in a second CFPS reaction mixture, and the method may include combining the first CFPS reaction mixture and the second CFPS reaction mixture to glycosylate the peptide or polypeptide sequence.

In some embodiments of the disclosed in vitro methods, the methods further include reacting the peptide comprising the N-linked Glc glycan with a second glycosyltransferase that is soluble and that catalyzes transfer to the N-linked glycan a monosaccharide (optionally wherein the monosaccharide is Glc, galactose (Gal), N-acetylgalactosamine (GalNAc), N-acetylglucosamine (GlcNAc), pyruvate, fucose (Fuc), sialic acid (Sia), a non-standard sugar such as an azido sugar including sialic acid functionalized at the C5 or C9 with an azido group position, sugars with alkyne, or strained alkynes/alkene functional groups sugars (including azido-sialic acid); sugars with thiol or maleimide groups; deoxysugars; PEGylated sugars; amino sugars; pre-assembled oligo- or polysaccharides containing natural and/or non-natural monomers; fluorinated sugars; and combinations thereof, wherein the glycosylation mixture comprises a Glc donor, a Gal donor, a GalNAc donor, a GlcNAc donor, a pyruvate donor, a fucose donor, a sialic acid donor, an azido-sialic acid donor, or a mixture thereof. The N-linked glycan then is glycosylated to provide an N-linked glycan comprising one or more moieties selected from Glc, Gal, GalNAc, GlcNAc, pyruvate, Fuc, and Sia (optionally to provide N-linked dextrose, N-linked lactose, or N-linked Glc-GalNAc), optionally wherein the second oligonucleotide transferase is expressed in a cell-free protein synthesis (CFPS) reaction mixture prior to performing glycosylation. Optionally, the peptide or polypeptide sequence may be expressed in a first CFPS reaction mixture, the NGT may be expressed in a second CFPS reaction mixture, and/or the second glycosyltransferase may be expressed in a third CFPS reaction mixture, and the method may include combining two or more of the first CFPS reaction mixture, the second CFPS reaction mixture, and/or the third reaction mixture to glycosylate the peptide or polypeptide sequence.

In some embodiments of the disclosed in vitro methods, the methods further include reacting the peptide comprising the glycan with a third glycosyltransferase that is soluble and that catalyzes transfer to the N-linked glycan a monosaccharide (optionally wherein the monosaccharide is Glc, Gal, GalNAc, GlcNAc, pyruvate, Fuc, Sia, or a non-standard sugar such as an azido sugar, wherein the glycosylation mixture comprises a Glc donor, a Gal donor, a GalNAc donor, a GlcNAc donor, a pyruvate donor, a fucose donor, a sialic acid donor, an azido-sialic acid donor, a non-natural sugar donor such as an azido sugar donor including a donor of sialic acid functionalized at the C5 or C9 with an azido group position, or a mixture thereof, and wherein the N-linked glycan further is glycosylated with one or more moieties selected from Glc, Gal, GalNAc, GlcNAc, pyruvate, Fuc, Sia and a non-standard sugar such as sugars with azido, alkyne, or strained alkynes/alkene functional groups sugars (including azido-sialic acid); sugars with thiol or maleimide groups; deoxysugars; PEGylated sugars; amino sugars; pre-assembled oligo- or polysaccharides containing natural and/or non-natural monomers; fluorinated sugars; and others. The N-linked glycan then is further glycosylated to provide an N-linked glycan comprising one or more moieties selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3Gal or GlcNAcβ1-4Galα1-3Gal). Optionally, the peptide or polypeptide sequence may be expressed in a first CFPS reaction mixture, the NGT may be expressed in a second CFPS reaction mixture, the second glycosyltransferase may be expressed in a third CFPS reaction mixture, and/or the third glycosyltransferase may be expressed in a fourth CFPS reaction mixture, and the method may include combining two or more of the first CFPS reaction mixture, the second CFPS reaction mixture, the third reaction mixture, and/or the fourth reaction mixture to glycosylate the peptide or polypeptide sequence.

Suitable CFPS reaction mixtures for the disclosed methods may include prokaryotic CFPS reaction mixtures. In some embodiments, suitable CFPS reaction mixtures may include prokaryotic CFPS reaction mixtures comprising a lysate prepared from Escherichia coli.

In some embodiments, the CFPS reaction mixtures for use in the disclosed methods may include and/or may express a peptide or polypeptide sequence for glycosylation in the disclosed methods (e.g., a peptide or polypeptide sequence comprising an amino acid motif N-X-S/T or a peptide or polypeptide sequence engineered to comprise an amino acid motif N-X-S/T where the amino acid motif N-X-S/T is not naturally present in the peptide or polypeptide sequence).

In some embodiments, the disclosed methods may include and/or may utilize a bacterial NGT optionally selected from the group consisting ofActinobacillus pleuropneumoniae (ApNGT) (SEQ ID NO:1) or a derivative thereof having the following substitution Q469A, Escherichia coli NGT (EcNGT) (SEQ ID NO:3), Haemophilus influenza NGT (HiNGT) (SEQ ID NO:5), Mannheimia haemolytica NGT (MhNGT) (SEQ ID NO:7), Haemophilus dureyi NGT (HdNGT) (SEQ ID NO:9), Bibersteinia trehalosi NGT (BtNGT) (SEQ ID NO:11), Aggregatibacter aphrophilus NGT (AaNGT) (SEQ ID NO:13), Yersinia enterocolitica NGT (YeNGT) (SEQ ID NO:15), Yersinia pestis NGT (YpNGT) (SEQ ID NO:17), and Kingella kingae NGT (KkNGT) (SEQ ID NO:19). Optionally, the bacterial NGT may be a modified bacterial NGT having one or more mutations relative to a wild-type bacterial NGT.

In some embodiments, the disclosed methods may include or utilize a modified NGT such as a modified bacterial NGT comprising one or more mutations, for example, mutations that change peptide acceptor specificity and/or increase enzymatic turnover rates. (See Song et al., “Production of homogeneous glycoprotein with multisite modifications by an engineered N-glycosyltransferase mutant,” J. Biol. Chem., Apr. 5, 2017, 292, 8856-8863, the content of which is incorporated herein by reference in its entirety). In some embodiments, the modified bacterial NGT is a modified ApNGT having a substitution at Q469 for example where Q469 is replaced with an amino acid X, where X is selected from S, T, N, C, G, P, A, I, L, M, V (see, e.g., SEQ ID NO:2 having Q469A). In some embodiments, the modified bacterial NGT is a modified EcNGT having a substitution at F482 where F482 is replaced with an amino acid X, where X is selected from S, T, N, C, G, P, A, I, L, M, V (see, e.g., SEQ ID NO:4, having F482A). In some embodiments, the modified bacterial NGT is a modified HiNGT having a substitution at Q495 where Q495 is replaced with an amino acid X, where X is selected from S, T, N, C, G, P, A, I, L, M, V (see, e.g, SEQ ID NO:6 having Q495A). In some embodiments, the modified bacterial NGT is a modified MhNGT having a substitution at Q469 where Q469 is replaced with an amino acid X, where X is selected from S, T, N, C, G, P, A, I, L, M, V (see, e.g, SEQ ID NO:8 having Q469A). In some embodiments, the modified bacterial NGT is a modified HdNGT having a substitution at Q468 where Q468 is replaced with an amino acid X, where X is selected from S, T, N, C, G, P, A, I, L, M, V (see, e.g, SEQ ID NO:10 having Q468A). In some embodiments, the modified bacterial NGT is a modified BtNGT having a substitution at Q471 where Q471 is replaced with an amino acid X, where X is selected from S, T, N, C, G, P, A, I, L, M, V (see, e.g, SEQ ID NO:12 having Q471A). In some embodiments, the modified bacterial NGT is a modified AaNGT having a substitution at Q468 where Q468 is replaced with an amino acid X, where X is selected from S, T, N, C, G, P, A, I, L, M, V (see, e.g, SEQ ID NO:14 having Q468A). In some embodiments, the modified bacterial NGT is a modified YeNGT having a substitution at F466 where F466 is replaced with an amino acid X, where X is selected from S, T, N, C, G, P, A, I, L, M, V (see, e.g, SEQ ID NO:16 having F466A). In some embodiments, the modified bacterial NGT is a modified YpNGT having a substitution at F466 where F466 is replaced with an amino acid X, where X is selected from S, T, N, C, G, P, A, I, L, M, V (see, e.g, SEQ ID NO:18 having F466A). In some embodiments, the modified bacterial NGT is a modified KkNGT having a substitution at Q474 where Q474 is replaced with an amino acid X, where X is selected from S, T, N, C, G, P, A, I, L, M, V (see, e.g, SEQ ID NO:20 having Q474A).

In some embodiments, the disclosed methods may include and/or may utilize a glycosyltransferase having the amino acid sequence of any of SEQ ID NOs:1, 3, 5, 7, 9, 11, 13, 15, 17, or 19 or having a least 50%, 60%, 70%, 80%, 90%, 95%, 96%, 97%, 98%, or 99% sequence identity to any of SEQ ID NOs:1, 3, 5, 7, 9, 11, 13, 15, 17, or 19, or the first glycosyltransferase is a modified bacterial N-linked glycosyltransferase (NGT) having the amino acid sequence of any of SEQ ID NOs:2, 4, 6, 8, 10, 12, 14, 16, 18, or 20, or having a least 50%, 60%, 70%, 80%, 90%, 95%, 96%, 97%, 98%, or 99% sequence identity to any of SEQ ID NOs:2, 4, 6, 8, 10, 12, 14, 16, 18, or 20.

In some embodiments, the CFPS reaction mixtures for use in the disclosed methods may include and/or may express a glycosyltransferase for use in the disclosed methods such as an α1-6 glucosyltransferase, a β1-4 galactosyltransferase, or a β1-3 N-acetylgalactosamine transferase, optionally selected from the group consisting of Actinobacillus pleuropneumoniae α1-6 glucosyltransferase (Apα1-6), Neisseria gonorrhoeae β1-4 galactosyltransferase LgtB (NgLGtB), Neisseria meningitidis β1-4 galactosyltransferase LgtB (NmLGtB), and Bacteriodes fragilis β1-3 N-acetylgalactosamine transferase (BfGalNAcT).

In some embodiments, the CFPS reaction mixtures for use in the disclosed methods may include and/or may express The CFPS reaction mixtures may include and/or may express a β1-3 N-acetylglucosamine transferase, a pyruvyltransferase, an α1-3 fucosyltransferase, an α1-2 fucosyltransferase, an α1-4 galactosyltransferase, an α1-3 galactosyltransferase, an α2-6 sialyltransferase, an α2-3,6 sialyltransferase, an α2-3 sialyltransferase, or an α2-3,8 sialyltransferase, optionally selected from the group consisting of Neisseria gonorrhoeae β1-3 N-acetylglucosamine transferase (NgLgtA), Schizosaccharomyces pombe pyruvyltransferase (SpPvg1),Helicobacter pylori α1-3 fucosyltransferase (HpFutA), Helicobacter pylori α1-2 fucosyltransferase (HpFutC), Neisseria meningitidis α1-4 galactosyltransferase (NmLgtC), Bos taurus α1-3 galactosyltransferase (BtGGTA), Homo sapiens α2-6 sialyltransferase (HsSIAT1), Photobacterium damselae α2-6 sialyltransferase (PdST6), Photobacterium leiognathid α2-6 sialyltransferase (P1ST6), Pasteurella multocida α2-3,6 sialyltransferase (PmST3,6), Vibrio sp JT-FAJ-16 α2-3 sialyltransferase (VsST3), Photobacterium phosphoreum α2-3 sialyltransferase (PpST3), Campylobacter jejuni α2-3 sialyltransferase (CjCST-I), and Campylobacter jejuni α2-3,8 sialyltransferase (CjCST-II).

Also disclosed are peptides, polypeptide, or proteins comprising an N-linked glycan and prepared by any of the disclosed methods. In some embodiments, the N-linked glycan comprises a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3Gal or GlcNAcβ1-4Galα1-3Gal), optionally wherein the peptide, polypeptide, or protein is utilized or formulated as a therapeutic agent or a vaccine.

### Applications

Applications of the disclosed technology include, but are not limited to: (i) High-throughput testing of glycosyltransferase enzyme specificities and activities to choose optimum enzymes variants and combinations for synthesis in living cells or on-demand manufacturing; (ii) the use of discovered biosynthetic pathways described herein for on-demand synthesis of glycoproteins in which the glycosylation enzymes and target protein are all synthesized in one-pot and use supplemented with sugar donors; (iii) The use of discovered biosynthetic pathways described herein for production of glycoprotein therapeutics, vaccines, diagnostics or analytical standards in vitro or in living E. coli; (iv) The use of discovered biosynthetic pathways described herein to produce more homogeneous glycoprotein therapeutics, vaccines, diagnostics or analytical standards in vitro or in living E. coli; (v) The synthesis of vaccine proteins modified with immunostimulatory glycosylation structures using the in vitro pathway described in this work for on-demand biomanufacturing in vitro or for production of glycoproteins in living cells; (vi) The synthesis of allergy vaccines with immunomodulatory minimal sialic acid motifs in in vitro or in living cells; (vii) The synthesis of therapeutic proteins (including antibodies) modified with sialic acid containing glycans using the pathways described in this work for on-demand biomanufacturing in vitro or for production of glycoproteins in living cells; (viii) Cell-free biosynthesis of vaccines with galactose-α1,3-galactose (alpha-galactose or alpha-gal); (ix) Simplification of production of tolerogenic allergy vaccines by clicking on lipophilic groups that are known to interact with Siglec receptors on T-regulatory cells; and (x) Simplification of the production of PEGylated proteins from bacteria (no purified enzymes and orthogonal to all OTS strategies and standard amino acid chemistries).

### Advantages

Advantages of the disclosed technology may include, but are not limited to, one or more of the following aspects. The glycosylation pathways described herein provide several new routes to therapeutically relevant glycans from an Asn-linked glucose residue installed by an N-linked glycosyltransferase (NGT). Glycosylation pathways beginning with NGT installation of monosaccharides in the cytoplasm have several advantages over existing chemical conjugation or oligosaccharyltransferase glycosylation methods as they allow for efficient glycosylation of polypeptides without a eukaryotic host, transport across cellular membranes, complex chemical synthesis or lipid-bound substrates and enzymes. The peptide acceptor specificity of NGT is also very well understood. Ultimately these pathways can be used to produce therapeutically relevant glycoproteins in vitro or in living cells.

There are currently close constraints on the diversity of vaccine proteins or glycoconjugate carrier proteins that can be used because most proteins do not elicit a substantial immune response. By modifying vaccine proteins with an adjuvant glycan using the method described in this work, it may be possible to improve existing vaccines or enable the use of a wider array of vaccine proteins or glycoconjugate carrier proteins.

Many glycoprotein production systems result in heterogeneity or unwanted glycoforms. By defining glycosylation systems in bacteria which do not contain endogenous glycosylation systems or by defining reaction conditions in vitro, the methods and pathways described here could enable the production or more homogeneous glycoprotein therapeutics.

The rational design and engineering of glycoproteins remains limited by the throughput of current methods for glycoprotein biosynthetic pathway construction which require genetic manipulation, expression, and analysis of glycoproteins from living cells. The inventors' cell-free platform for synthesis and prototyping of protein glycosylation pathways allows for the rapid testing of new protein glycosylation pathways. This platform is amenable to massively parallel synthesis and assembly of glycosylation pathways, facile manipulation of reaction conditions, and automated liquid handling. Once prototyped, these pathways can be applied to the production of glycoproteins in vitro or in vivo.

Although cell-free biosynthetic pathway prototyping has been applied to the synthesis of small molecules and some single-enzyme glycosylation processes have been recapitulated in vitro, this is the first application of cell-free biosynthetic prototyping to multienzyme protein glycosylation systems.

### Technical Field

The technical field relates to development of novel, multi-enzyme protein glycosylation pathways using cell-free protein synthesis.

### Technical Problem Solved by the Technology

Most methods for glycoprotein synthesis use native pathways within eukaryotic organisms, usually CHO cells. However, these methods result in glycan heterogeneity, limit the choice of biomanufacturing hosts, and provide limited control over glycosylation structures which are known to profoundly affect protein properties, especially for protein therapeutics. These limitations have motivated the development of engineered or synthetic glycosylation systems, either by cellular engineering of eukaryotes (yeast or CHO cells), bacterial systems, or in vitro. Among these, synthetic glycosylation systems constructed in bacteria or in vitro offer the opportunity to most closely control glycosylation patterns and more rapidly develop more diverse glycosylation patterns. The use of bacterial hosts also enables more cost-effective biomanufacturing.

Several bacterial systems have been developed to produce protein vaccines or glycosylated therapeutics. However, the development of these synthetic glycosylation systems remains slow as it requires the construction and testing sets of enzymes (biosynthetic pathways) in living cells. Consequently, the glycosylation structures produced in bacterial are usually limited to those that can be synthesized by expressing whole operons found in nature, which severely constrains the diversity of structures that can be constructed and therefore the diversity of applications to which this technology can be applied. The inventors' cell-free glycosylation prototyping technology presents a way to rapidly synthesize and test synthetic glycosylation systems. Using this technology, the inventors have discovered several novel biosynthetic pathways that can be used for production of glycoprotein therapeutics, vaccines, and analytical standards in vitro or in living cells.

A key differentiating factor of the biosynthetic pathways that the inventors developed compared to existing work is that they use a soluble, highly active N-linked glycosyltransferase (NGT) to install a single sugar onto proteins and then elaborate this single sugar into a wide array of therapeutically relevant glycans. This is in contrast to most existing work that use oligosaccaryltransferases (OSTs) to conjugate lipid linked sugar donors en bloc onto proteins. The highly active and soluble nature of NGT lends a major technical advantage for synthesis of glycoproteins in living cells or in vitro. However, the use of NGTs for the modification of heterologous proteins has been limited, likely due to a lack of known biosynthetic pathways to elaborate the single sugar installed to therapeutically relevant glycosylation structures. So far, only one work (Keyes et al., Metabolic Engineering, 2017) has demonstrated the entirely biosynthetic use of NGT to produce a therapeutically relevant glycan (polysialic acid). The inventors' work provides a variety of new glycosylation structures with much broader applicability, such as the production of protein vaccines with immunostimulatory glycosylation structures.

In addition to production of proteins in living systems, others have used total chemical synthesis to construct defined glycoproteins by solid-phase peptide synthesis (SPPS). While useful for small glycopeptides, this method becomes much more difficult for larger proteins and is unlikely to be commercially viable for the production of whole glycoproteins proteins. Still others have used chemical synthesis to produce defined glycans and then transfer these glycans to whole protein produced in cells. Indeed this has also been employed in combination with modification of proteins with NGT (Lomino et al., Bioorg Med Chem., 2013). While more promising for commercial applications than total chemical synthesis, this method still requires laborious and expensive chemical steps to produce the glycans. The inventors' technology uses enzymes to build glycans directly on proteins, and is amenable to total biosynthetic production in living cells or in one-pot cell-free systems, presenting a cheaper, more commercially viable approach.

While other methods have incorporated azido sugars in bacteria, they have only used this for visualization and study rather than engineering modification of therapeutics.

### Commercialization

The disclosed technology may be commercialized in manners that include, but are not limited to the following. The inventors' cell-free platform allows for the prototyping of multi-enzyme glycosylation systems in vitro, allowing for the more rapid development of biosynthetic pathways for protein glycosylation. Several pathways discovered in the inventors' work could solve existing problems with synthesis of glycoproteins in mammalian cells as they would allow for the production of therapeutically relevant glycoproteins in bacteria for large-scale production or in vitro for research or on-demand synthesis applications. Specific application areas include protein vaccines with antigenic or immunomodulatory glycans as well as protein therapeutics with extended half-lives or increased stability.

### Value

The value of the disclosed technology includes, but is not limited to the following. The inventors have described the use of a cell-free system to prototype and discover novel glycosylation biosynthetic pathways. Biopharmaceutical firms may license this technology to pursue cell-free prototyping projects towards certain glycoproteins of their choice, or directly use the biosynthetic pathways discovered in this work to produce protein therapeutics and vaccines with enhanced properties (notably the installation of sialic acids on protein therapeutics or vaccines and the installation of alpha-galactose immunostimulatory motifs on protein vaccines) in vitro or in living cells. The lipid-independent nature of the biosynthetic pathways discovered in this work makes them particularly attractive for synthesis of glycoprotein therapeutics in vitro or in the bacterial cytoplasm. These high-titer, rapid expression systems could allow glycoprotein therapeutics to be developed and produced more quickly and at lower cost.

### Miscellaneous

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Embodiments

1. Biosynthetic pathways (sets of enzymes) as well as modes of synthesis of all glycoforms described in attached manuscript.

2. Glycoforms prepared through the biosynthetic pathways of embodiment 1.

3. Expression of enzymatic pathways in embodiment 1 in a living cell, in particular, the demonstrated embodiments of glycans terminated in alpha-gal and sialic acids. In some embodiments, an N-linked glucose and/or an N-linked lactose is provided.

4. Use of polypeptide sequences and/or enzymes in embodiment 1 as a means of glycosylation in vitro.

5. Cell-free biosynthesis of glycoproteins with biosynthetic pathways described in any of the foregoing embodiments.

6. Cell-free biosynthesis of glycoproteins with biosynthetic pathways described in any of the foregoing embodiments in a freeze-dried format.

7. Cell-free method for rapid prototyping of protein glycosylation pathways to design biosynthetic pathways in vivo. This method comprising one or more of the following steps: (i) Use of an NGT to install a priming glucose onto a protein; (ii) Combinatorial assembly of pathways in cell-free systems by mixing-and-matching cell lysates enriched with pathway enzymes; (iii) Rapid in vitro glycosylation pathway assembly; and (iv) Transfer of pathways identified for making glycoproteins in in vitro and in vivo production platforms.

8. The embodiment of claim 7 where enzymes are enriched in lysates by cell-free protein synthesis.

9. The embodiment of claim 7 where enzymes are enriched by overexpression in a lysate source strain

### US Published Applications and Patents

US2004/0171826; US2004/0018590; US2004/0230042; US2005/0260729; US2005/0170452; US2005/0208617; US2005/0170452; US2006/0148035; US2006/040353; US2006/0286637; US2006/0177898; US2006/0211085; US2006/0024292; US2006/0024304; US2006/0234345; US2006/0252672; US2006/0257399; US2006/0286637; US2006/0029604; US2006/0034828; US2007/0026485; US2007/0178551; US2007/0178551; US2007/0037248; US2008/0274498; US2008/0199942; US2009/0155847; US2009/0209024; US2010/0279356; US2010/0062516; US2010/0062523; US2010/0021991; US2010/0184143; US2010/0016561; US2011/0053214; US2012/0052530; US2012/0064568; US2013/021706; US2013/0018177; US2014/0194345; US2015/0079633; US2015/0203890; US2015/0152427; US2015/0190492; US2016/0362708; US2016/0068880; US2018/0016612; US2018/0354997; U.S. Pat. Nos. 8,703,471; and 8,999,668; the contents of which are incorporated herein by reference in their entireties.

### International and Foreign Applications and Patents

WO2003056914; WO2004035605; WO2005090552; WO2006102652; WO2006119987; WO2007101862; WO2017117539; WO2007120932; CN105505959; CN107090442; and CN107034202; the contents of which are incorporated herein by reference in their entireties.

### Non-Patent References

Xu, Y. et al. A novel enzymatic method for synthesis of glycopeptides carrying natural eukaryotic N-glycans. Chemical Communications 53, 9075-9077 (2017).

Kong, Y. et al. N-Glycosyltransferase from Aggregatibacter aphrophilus synthesizes glycopeptides with relaxed nucleotide-activated sugar donor selectivity. Carbohydrate Research 462, 7-12 (2018).

Keys, T. G. et al. A biosynthetic route for polysialylating proteins in Escherichia coli. Metabolic Engineering 44, 293-301 (2017).

Keys, T. G. & Aebi, M. Engineering protein glycosylation in prokaryotes. Current Opinion in Systems Biology 5, 23-31 (2017).

Cuccui, J. et al. The N-linking glycosylation system from Actinobacillus pleuropneumoniae is required for adhesion and has potential use in glycoengineering. Open biology 7 (2017).

Song, Q. et al. Production of homogeneous glycoprotein with multi-site modifications by an engineered N-glycosyltransferase mutant. Journal of Biological Chemistry (2017).

Naegeli, A. et al. Substrate Specificity of Cytoplasmic N-Glycosyltransferase. Journal of Biological Chemistry 289, 24521-24532 (2014).

Naegeli, A. et al. Molecular analysis of an alternative N-glycosylation machinery by functional transfer from Actinobacillus pleuropneumoniae to Escherichia coli. The Journal of biological chemistry 289, 2170-2179 (2014).

Schwarz, F., Fan, Y.-Y., Schubert, M. & Aebi, M. Cytoplasmic N-Glycosyltransferase of Actinobacillus pleuropneumoniae Is an Inverting Enzyme and Recognizes the NX(S/T) Consensus Sequence. Journal of Biological Chemistry 286, 35267-35274 (2011).

Jaroentomeechai, T. et al. Single-pot glycoprotein biosynthesis using a cell-free transcription-translation system enriched with glycosylation machinery. Nature Communications 9, 2686 (2018).

Schoborg, J. A. et al. A cell-free platform for rapid synthesis and testing of active oligosaccharyltransferases. Biotechnology and bioengineering (2017).

Guarino, C., & DeLisa, M. P. (2012). A prokaryote-based cell-free translation system that efficiently synthesizes glycoproteins. Glycobiology, 22(5), 596-601.

Lizak, C., Fan, Y. -Y., Weber, T. C. & Aebi, M. N-Linked Glycosylation of Antibody Fragments in Escherichia coli. Bioconjugate chemistry 22, 488-496 (2011).

Karim, A. S. & Jewett, M. C. A cell-free framework for rapid biosynthetic pathway prototyping and enzyme discovery. Metabolic Engineering 36, 116-126 (2016).

Huai, G., Qi, P., Yang, H. & Wang, Y. I. Characteristics of α-Gal epitope, anti-Gal antibody, a1,3 galactosyltransferase and its clinical exploitation (Review). International journal of molecular medicine 37, 11-20 (2016).

Abdel-Motal, U. M. et al. Increased immunogenicity of HIV-1 p24 and gp120 following immunization with gp120/p24 fusion protein vaccine expressing alpha-gal epitopes. Vaccine 28, 1758-1765 (2010).

Meuris, L. et al. GlycoDelete engineering of mammalian cells simplifies N-glycosylation of recombinant proteins. Nat Biotech 32, 485-489 (2014).

The contents of the afore-cited non-patent reference are incorporated herein by reference in their entireties.

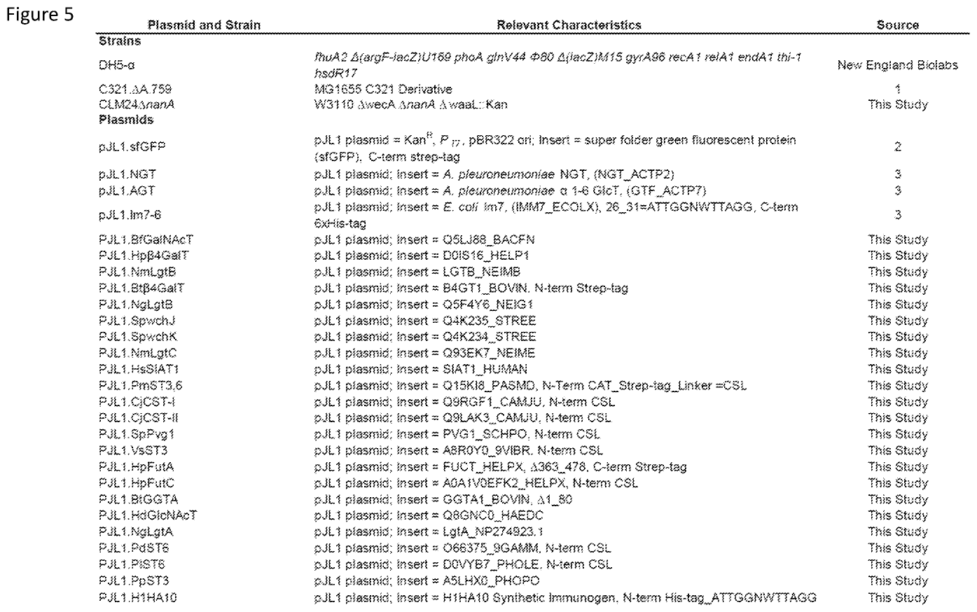

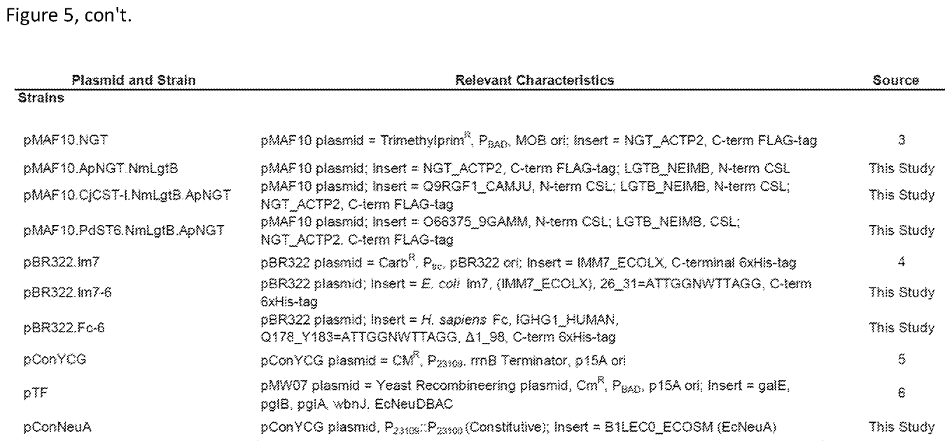

### References Cited in FIGS. 5, 6 and 20.

1. Martin, R. W. et al. Cell-free protein synthesis from genomically recoded bacteria enables multisite incorporation of noncanonical amino acids. Nature Communications 9, 1203 (2018).

2. Bundy, B. C. & Swartz, J. R. Site-Specific Incorporation of p-Propargyloxyphenylalanine in a Cell-Free Environment for Direct Protein-Protein Click Conjugation. Bioconjugate chemistry 21, 255-263 (2010).

3. Kightlinger, W. et al. Design of glycosylation sites by rapid synthesis and analysis of glycosyltransferases. Nature Chemical Biology 14, 627-635 (2018).

4. Ollis, A. A., Zhang, S., Fisher, A. C. & DeLisa, M. P. Engineered oligosaccharyltransferases with greatly relaxed acceptor-site specificity. Nature Chemical Biology 10, 816-822 (2014).

5. Glasscock, C. J. et al. A flow cytometric approach to engineering Escherichia coli for improved eukaryotic protein glycosylation. Metabolic Engineering 47, 488-495 (2018).

6. Valentine, Jenny L. et al. Immunization with Outer Membrane Vesicles Displaying Designer Glycotopes Yields Class-Switched, Glycan-Specific Antibodies. Cell Chemical Biology 23, 655-665 (2016).

7. Naegeli, A. et al. Substrate Specificity of Cytoplasmic N-Glycosyltransferase. Journal of Biological Chemistry 289, 24521-24532 (2014).

8. Schwarz, F., Fan, Y.-Y., Schubert, M. & Aebi, M. Cytoplasmic N-Glycosyltransferase of Actinobacillus pleuropneumoniae Is an Inverting Enzyme and Recognizes the NX(S/T) Consensus Sequence. Journal of Biological Chemistry 286, 35267-35274 (2011).

9. Park, J. E., Lee, K. Y., Do, S. I. & Lee, S. S. Expression and characterization of beta-1,4-galactosyltransferase from Neisseria meningitidis and Neisseria gonorrhoeae. Journal of biochemistry and molecular biology 35, 330-336 (2002).

10. Peng, W. et al. Helicobacter pylori β1,3-N-acetylglucosaminyltransferase for versatile synthesis of type 1 and type 2 poly-LacNAcs on N-linked, 0-linked and I-antigen glycans. Glycobiology 22, 1453-1464 (2012).

11. Ramakrishnan, B. & Qasba, P. K. Crystal structure of lactose synthase reveals a large conformational change in its catalytic component, the beta1,4-galactosyltransferase-I. Journal of Molecular Biology 310, 205-218 (2001).

12. Aanensen, D. M., Mavroidi, A., Bentley, S. D., Reeves, P. R. & Spratt, B. G. Predicted Functions and Linkage Specificities of the Products of the Streptococcus pneumoniae Capsular Biosynthetic Loci. Journal of bacteriology 189, 7856-7876 (2007).

13. Ban, L. et al. Discovery of glycosyltransferases using carbohydrate arrays and mass spectrometry. Nature Chemical Biology 8, 769-773 (2012).

14. Blixt, 0., van Die, I., Norberg, T. & van den Eijnden, D. H. High-level expression of the Neisseria meningitidis lgtA gene in Escherichia coli and characterization of the encoded N-acetylglucosaminyltransferase as a useful catalyst in the synthesis of GlcNAcβ1→3Gal and GalNAcβ1→3Gal linkages. Glycobiology 9, 1061-1071 (1999).

15. Higuchi, Y. et al. A rationally engineered yeast pyruvyltransferase Pvg1p introduces sialylation-like properties in neo-human-type complex oligosaccharide. Scientific reports 6, 26349 (2016).

16. Sun, S., Scheffler, N. K., Gibson, B. W., Wang, J. & Munson Jr., R. S. Identification and Characterization of the N-Acetylglucosamine Glycosyltransferase Gene of Haemophilus ducreyi. Infection and immunity 70, 5887-5892 (2002).

17. Wang, G., Ge, Z., Rasko, D. A. & Taylor, D. E. Lewis antigens in Helicobacter pylori: biosynthesis and phase variation. Molecular Microbiology 36, 1187-1196 (2000).

18. Persson, K. et al. Crystal structure of the retaining galactosyltransferase LgtC from Neisseria meningitidis in complex with donor and acceptor sugar analogs. Nature Structural Biology 8, 166 (2001).

19. Fang, J. et al. Highly Efficient Chemoenzymatic Synthesis of α-Galactosyl Epitopes with a Recombinant α(1→3)-Galactosyltransferase. Journal of the American Chemical Society 120, 6635-6638 (1998).

20. Hidari, K. I. et al. Purification and characterization of a soluble recombinant human ST6Gal I functionally expressed in Escherichia coli. Glycoconjugate Journal 22, 1-11 (2005).

21. Yamamoto, T. Marine Bacterial Sialyltransferases. Marine Drugs 8, 2781 (2010).

22. Chiu, C. P .C. et al. Structural Analysis of the α-2,3-Sialyltransferase Cst-I from Campylobacter jejuni in Apo and Substrate-Analogue Bound Forms. Biochemistry 46, 7196-7204 (2007).

23. Keys, T. G. et al. A biosynthetic route for polysialylating proteins in Escherichia coli. Metabolic Engineering 44, 293-301 (2017).

24. Kim, D. M. & Swartz, J. R. Efficient production of a bioactive, multiple disulfide-bonded protein using modified extracts of Escherichia coli. Biotechnology and bioengineering 85, 122-129 (2004).

The contents of the afore-cited non-patent reference are incorporated herein by reference in their entireties.

### Illustrative Embodiments

The following embodiments are illustrative and should not be interpreted to limit the scope of the claimed subject matter.

Embodiment 1. A cell-free system for glycosylating a peptide or polypeptide sequence in vitro, the peptide or polypeptide sequence comprising an asparagine residue and the system comprising as components: (i) a glycosyltransferase which is a soluble N-linked glycosyltransferase (NGT) that catalyzes transfer to an amino group of the asparagine residue a monosaccharide (optionally wherein the monosaccharide is glucose (Glc)) to provide an N-linked glycan, or an expression vector that expresses the NGT in a cell-free protein synthesis (CFPS) reaction mixture; (ii) a glycosylation mixture comprising a monosaccharide donor (optionally a Glc donor); wherein the peptide or polypeptide sequence is glycosylated in the glycosylation mixture in vitro to provide a peptide or polypeptide sequence comprising the N-linked glycan (optionally an N-linked Glc).

2. The system of claim 1, further comprising as a component: (iii) a second glycosyltransferase that is soluble and catalyzes transfer to the N-linked glycan a monosaccharide (optionally wherein the monosaccharide is Glc, galactose (Gal), N-acetylgalactosamine (GalNAc), N-acetylglucosamine (GlcNAc), pyruvate, fucose (Fuc), sialic acid (Sia)), or an expression vector that expresses the second glycosyltransferase in a cell-free protein synthesis (CFPS) reaction mixture; wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan is glycosylated with one or more moieties selected from Glc, Gal, GalNAc, GlcNAc, pyruvate, Fuc, Sia, and azido-Sia (optionally to provide N-linked dextrose, N-linked lactose, or N-linked Glc-GalNAc).

3. The system of claim 2 further comprising as a component: (iv) a third glycosyltransferase that is soluble and that catalyzes transfer to the N-linked glycan a monosaccharide (optionally wherein the monosaccharide is Glc, Gal, GalNAc, GlcNAc, pyruvate, Fuc, Sia, or combinations thereof), or an expression vector that expresses the third glycosyltransferase in a cell-free protein synthesis (CFPS) reaction mixture; wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan further is glycosylated with one or more moieties selected from Glc, Gal, GalNAc, GlcNAc, pyruvate, Fuc, Sia, and azido-Sia (optionally to provide an N-linked glycan comprising one or more moieties selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′ -fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3Gal or GlcNAcβ1-4Galα1-3 Gal)).

4. The system of any of the foregoing claims, wherein the system comprises a cell-free protein synthesis (CFPS) reaction mixture and one or more of the first glycosyltransferase, the second glycosyltransferase, and the third glycosyltransferase are present or expressed in the CFPS reaction mixture.

5. The system of any of the foregoing claims, wherein the system comprises one or more cell-free protein synthesis (CFPS) reaction mixtures and one or more of the first glycosyltransferase, the second glycosyltransferase, and the third glycosyltransferase are present or expressed in the CFPS reaction mixtures and the one or more CFPS reaction mixtures are combined to provide the system.

6. The system of any of the foregoing claims, further comprising the peptide or polypeptide sequence or an expression vector that expresses the peptide or polypeptide sequence, optionally wherein the peptide or polypeptide sequence is provided or expressed in a cell-free protein synthesis (CFPS) reaction mixture.

7. The system of any of the foregoing claims, wherein the CFPS reaction mixture is a prokaryotic CFPS reaction mixture.

8. The system of any of the foregoing claims, wherein the CFPS reaction mixture is a prokaryotic CFPS reaction mixture comprising a lysate prepared from Escherichia coli.

9. The system of any of the foregoing claims, wherein optionally the first glycosyltransferase is a bacterial N-linked glycosyltransferase (NGT), optionally wherein the bacterial NGT is a bacterial NGT selected from the group consisting of Actinobacillus pleuropneumoniae (ApNGT), Escherichia coli NGT (EcNGT), Haemophilus influenza NGT (HiNGT), Mannheimia haemolytica NGT (MhNGT), Haemophilus dureyi NGT (HdNGT), Bibersteinia trehalosi NGT (BtNGT), Aggregatibacter aphrophilus NGT (AaNGT), Yersinia enterocolitica NGT (YeNGT), Yersinia pestis NGT (YpNGT), and Kingella kingae NGT (KkNGT) or a modified form thereof.

10. The system of any of the foregoing claims, wherein the first glycosyltransferase is a bacterial N-linked glycosyltransferase (NGT) having the amino acid sequence of any of SEQ ID NOs:1, 3, 5, 7, 9, 11, 13, 15, 17, or 19 or having a least 50%, 60%, 70%, 80%, 90%, 95%, 96%, 97%, 98%, or 99% sequence identity to any of SEQ ID NOs:1, 3, 5, 7, 9, 11, 13, 15, 17, or 19, or the first glycosyltransferase is a modified bacterial N-linked glycosyltransferase (NGT) having the amino acid sequence of any of SEQ ID NOs:2, 4, 6, 8, 10, 12, 14, 16, 18, or 20, or having a least 50%, 60%, 70%, 80%, 90%, 95%, 96%, 97%, 98%, or 99% sequence identity to any of SEQ ID NOs:2, 4, 6, 8, 10, 12, 14, 16, 18, or 20.

11. The system of any of the foregoing claims, wherein optionally the second glycosyltransferases is an α1-6 glucosyltransferase, a β1-4 galactosyltransferase, or a β1-3 N-acetylgalactosamine transferase, and optionally wherein the second glycosyltransferase is selected from the group consisting of Actinobacillus pleuropneumoniae α1-6 glucosyltransferase (Apα1-6), Neisseria gonorrhoeae β1-4 galactosyltransferase LgtB (NgLGtB), Neisseria meningitidis β1-4 galactosyltransferase LgtB (NmLGtB), and Bacteriodes fragilis β1-3 N-acetylgalactosamine transferase (BfGalNAcT).

12. The system of any of the foregoing claims, wherein optionally the third glycosyltransferase is a β1-3 N-acetylglucosamine transferase, a pyruvyltransferase, an α1-3 fucosyltransferase, an α1-2 fucosyltransferase, an α1-4 galactosyltransferase, an α1-3 galactosyltransferase, an α2-6 sialyltransferase, an α2-3,6 sialyltransferase, an α2-3 sialyltransferase, or an α2-3,8 sialyltransferase, optionally wherein the third glycosyltransferase is selected from the group consisting of Neisseria gonorrhoeae β1-3 N-acetylglucosamine transferase (NgLgtA), Schizosaccharomyces pombe pyruvyltransferase (SpPvg1), Helicobacter pylori α1-3 fucosyltransferase (HpFutA), Helicobacter pylori α1-2 fucosyltransferase (HpFutC), Neisseria meningitidis α1-4 galactosyltransferase (NmLgtC), Bos taurus α1-3 galactosyltransferase (BtGGTA), Homo sapiens α2-6 sialyltransferase (HsSIAT1), Photobacterium damselae α2-6 sialyltransferase (PdST6), Photobacterium leiognathid α2-6 sialyltransferase (P1ST6), Pasteurella multocida α2-3,6 sialyltransferase (PmST3,6), Vibrio sp JT-FAJ-16 α2-3 sialyltransferase (VsST3), Photobacterium phosphoreum α2-3 sialyltransferase (PpST3), Campylobacter jejuni α2-3 sialyltransferase (CjCST-I), and Campylobacter jejuni α2-3,8 sialyltransferase (CjCST-II).

13. The system of any of the foregoing claims, wherein one or more components of the system are in a preserved form, optionally wherein one or more components of the system are freeze-dried.

14. A peptide or polypeptide sequence comprising an N-linked glycan (optionally prepared using any of the systems of the foregoing claims or components of the systems of the foregoing claims), the N-linked glycan comprising a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, an αGal epitope (e.g., Glcβ1-4Galα1-3Gal or GlcNAcβ1-4Galα1-3Gal), and Glc-Gal-azido-Sia, optionally wherein the peptide or polypeptide sequence is utilized or formulated as a therapeutic agent or a vaccine.

15. A modified cell that comprises or expresses one or more components of the systems of claims 1-13, optionally wherein the modified cell is a modified bacterial cell.

16. A method for preparing a glycosylated peptide or polypeptide sequence, the method comprising culturing the modified cell of claim 15, wherein the modified cell comprises or expresses a peptide or polypeptide sequence, an N-linked glycosyltransferase, and optionally one or more additional glycosyltransferases, and the peptide or polypeptide sequence is glycosylated in the modified bacterial cell.

17. A peptide or polypeptide sequence comprising an N-linked glycan (optionally prepared using the method of claim 16), the N-linked glycan comprising a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, an αGal epitope (e.g., Glcβ1-4Galα1-3Gal or GlcNAcβ1-4Galα1-3Gal), and Glc-Gal-azido-Sia, optionally wherein the peptide or polypeptide sequence is utilized or formulated as a therapeutic protein or vaccine.

18. A lysate prepared from the modified cell of claim 15, optionally wherein the lysate is suitable for use in a cell-free protein synthesis (CFPS) reaction.

19. A method for preparing a glycosylated peptide or polypeptide sequence in vitro, the method comprising reacting a peptide or polypeptide sequence comprising an asparagine residue in a glycosylation mixture comprising a monosaccharide donor (optionally wherein the monosaccharide donor is a glucose (Glc) donor, or is a monosaccharide) with a glycosyltransferase which is a soluble N-linked glycosyltransferase, (“N-glycotransferase,” “NGT”) that catalyzes transfer of the monosaccharide from the monosaccharide donor (optionally Glc from the Glc donor) to an amino group of the asparagine residue to provide an N-linked glycan (optionally an N-linked Glc), wherein the peptide or polypeptide sequence is glycosylated in the glycosylation mixture in vitro to provide a peptide or polypeptide sequence comprising the N-linked glycan (optionally an N-linked Glc), optionally wherein the peptide or polypeptide sequence, the NGT, or both are expressed in one or more cell-free protein synthesis (CFPS) reaction mixtures prior to performing glycosylation.

20. The method of claim 19, wherein the peptide or polypeptide sequence is expressed in a first CFPS reaction mixture, the NGT is expressed in a second CFPS reaction mixture, and the method comprises combining the first CFPS reaction mixture and the second CFPS reaction mixture.

21. The method of claim 19 or 20, further comprising reacting the peptide comprising the glycan with a second glycosyltransferase that is soluble and that catalyzes transfer to the N-linked glycan a monosaccharide (optionally wherein the monosaccharide is Glc, galactose (Gal), N-acetylgalactosamine (GalNAc), N-acetylglucosamine (GlcNAc), pyruvate, fucose (Fuc), sialic acid (Sia), or combinations thereof), wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan is glycosylated with one or more moieties selected from Glc, Gal, GalNAc, GlcNAc, pyruvate, Fuc, Sia, and azido-Sia (optionally to provide N-linked dextrose, N-linked lactose, or N-linked Glc-GalNAc), optionally wherein the second oligonucleotide transferase is expressed in a cell-free protein synthesis (CFPS) reaction mixture prior to performing glycosylation.

22. The method of claim 21, wherein the peptide or polypeptide sequence is expressed in a first CFPS reaction mixture, the NGT is expressed in a second CFPS reaction mixture, and the second glycosyltransferase is expressed in a third CFPS reaction mixture, and the method comprises combining two or more of the first CFPS reaction mixture, the second CFPS reaction mixture, and the third reaction mixture.

23. The method of claim 21 or 22, further comprising reacting the peptide comprising the glycan with a third glycosyltransferase that is soluble and that catalyzes transfer to the N-linked glycan a monosaccharide (optionally optionally wherein the monosaccharide is Glc, Gal, GalNAc, GlcNAc, pyruvate, Fuc, or Sia), wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan further is glycosylated with one or more moieties selected from Glc, Gal, GalNAc, GlcNAc, pyruvate, Fuc, Sia, azido-Sia (optionally to provide an N-linked glycan comprising one or more moieties selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, and an αGal epitope (e.g., Glcβ1-4Galα1-3 Gal or GlcNAcβ1-4Galα1-3 Gal)), and optionally wherein the second oligonucleotide transferase is expressed in a cell-free protein synthesis (CFPS) reaction mixture prior to performing glycosylation.

24. The method of claim 23, wherein the peptide or polypeptide sequence is expressed in a first CFPS reaction mixture, the NGT is expressed in a second CFPS reaction mixture, the second glycosyltransferase is expressed in a third CFPS reaction mixture, the third glycosyltransferase is expressed in a fourth CFPS reaction mixture, and the method comprises combining two or more of the first CFPS reaction mixture, the second CFPS reaction mixture, the third reaction mixture, and the fourth reaction mixture.

25. The method of any of claims 19-24, wherein the CFPS reaction mixture is a prokaryotic CFPS reaction mixture.

26. The method of any of claims 19-25, wherein the CFPS reaction mixture is a prokaryotic CFPS reaction mixture comprising a lysate prepared from Escherichia coli.

27. The method of any of claims 19-26, wherein optionally the first glycosyltransferase is a bacterial N-linked glycosyltransferase (NGT), and optionally the bacterial N-linked glycosyltransferase (NGT) is a bacterial NGT selected from the group consisting of Actinobacillus pleuropneumoniae (ApNGT), Escherichia coli NGT (EcNGT), Haemophilus influenza NGT (HiNGT), Mannheimia haemolytica NGT (MhNGT), Haemophilus dureyi NGT (HdNGT), Bibersteinia trehalosi NGT (BtNGT), Aggregatibacter aphrophilus NGT (AaNGT), Yersinia enterocolitica NGT (YeNGT), Yersinia pestis NGT (YpNGT), and Kingella kingae NGT (KkNGT), or a modified form thereof.

28. The method of any of claim 19-27, wherein the first glycosyltransferase is a bacterial N-linked glycosyltransferase (NGT) having the amino acid sequence of any of SEQ ID NOs:1, 3, 5, 7, 9, 11, 13, 15, 17, or 19 or having a least 50%, 60%, 70%, 80%, 90%, 95%, 96%, 97%, 98%, or 99% sequence identity to any of SEQ ID NOs:1, 3, 5, 7, 9, 11, 13, 15, 17, or 19, or the first glycosyltransferase is a modified bacterial N-linked glycosyltransferase (NGT) having the amino acid sequence of any of SEQ ID NOs:2, 4, 6, 8, 10, 12, 14, 16, 18, or 20, or having a least 50%, 60%, 70%, 80%, 90%, 95%, 96%, 97%, 98%, or 99% sequence identity to any of SEQ ID NOs:2, 4, 6, 8, 10, 12, 14, 16, 18, or 20.

29. The method of any of claims 19-28, wherein optionally the second glycosyltransferases is an α1-6 glucosyltransferase, a β1-4 galactosyltransferase, or a β1-3 N-acetylgalactosamine transferase, and optionally wherein the second glycosyltransferase is selected from the group consisting of Actinobacillus pleuropneumoniae α1-6 glucosyltransferase (Apα1-6), Neisseria gonorrhoeae β1-4 galactosyltransferase LgtB (NgLGtB), Neisseria meningitidis β1-4 galactosyltransferase LgtB (NmLGtB), and Bacteriodes fragilis β1-3 N-acetylgalactosamine transferase (BfGalNAcT).

30. The method of any of claims 19-29, wherein optionally the third glycosyltransferase is a β1-3 N-acetylglucosamine transferase, a pyruvyltransferase, an α1-3 fucosyltransferase, an α1-2 fucosyltransferase, an α1-4 galactosyltransferase, an α1-3 galactosyltransferase, an α2-6 sialyltransferase, an α2-3,6 sialyltransferase, an α2-3 sialyltransferase, or an α2-3,8 sialyltransferase, optionally wherein the third glycosyltransferase is selected from the group consisting of Neisseria gonorrhoeae β1-3 N-acetylglucosamine transferase (NgLgtA), Schizosaccharomyces pombe pyruvyltransferase (SpPvg1), Helicobacter pylori α1-3 fucosyltransferase (HpFutA), Helicobacter pylori α1-2 fucosyltransferase (HpFutC), Neisseria meningitidis α1-4 galactosyltransferase (NmLgtC), Bos taurus α1-3 galactosyltransferase (BtGGTA), Homo sapiens α2-6 sialyltransferase (HsSIAT1), Photobacterium damselae α2-6 sialyltransferase (PdST6), Photobacterium leiognathid α2-6 sialyltransferase (P1ST6), Pasteurella multocida α2-3,6 sialyltransferase (PmST3,6), Vibrio sp JT-FAJ-16 α2-3 sialyltransferase (VsST3), Photobacterium phosphoreum α2-3 sialyltransferase (PpST3), Campylobacter jejuni α2-3 sialyltransferase (CjCST-I), and Campylobacter jejuni α2-3,8 sialyltransferase (CjCST-II).

31. A peptide or polypeptide sequence comprising an N-linked glycan prepared by any of the methods of claims 19-30, optionally wherein the N-linked glycan comprises a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, an αGal epitope (e.g., Glcβ1-4Galα1-3Gal or GlcNAcβ1-4Galα1-3Gal), and Glc-Gal-azido-Sia, optionally wherein the peptide or polypeptide sequence is utilized or formulated as a therapeutic agent or a vaccine.

32. A protein synthesized by any of the methods of claims 19-30 and utilized or formulated as a therapeutic or vaccine, optionally wherein the protein comprises an N-linked glycan and the N-linked glycan comprises a moiety selected from the group consisting of sialylated forms of lactose (e.g., mono-sialylated forms of lactose such as 3′-siallylactose, 6′-siallylactose, and di-sialylated forms of lactose), fucosylated forms of lactose (e.g., mono-fucosylated forms of lactose such as 2′-fucosyllactose (Glcβ1-4Galα1-2Fuc) and 3′-fucosylactose (i.e., (Glcβ1-4Galα1-23Fuc), and di-fucosylated forms of lactose), sialylated forms of LacNAc (e.g., mono-sialylated forms of LacNAc and di-sialylated forms of LacNAc), fucosylated forms of LacNAc (e.g., mono-fucosylated forms of LacNAc and di-fucosylated forms of LacNAc), pyruvylated lactose or pyruvylated LacNAc, an αGal epitope (e.g., Glcβ1-4Galα1-3Gal or GlcNAcβ1-4Galα1-3Gal), and Glc-Gal-azido-Sia.

### EXAMPLES

The following Examples are illustrative and are not intended to limit the scope of the claimed subject matter.

### Example 1

### A Modular Cell-Free Platform for Production of Glycoproteins and Identification of Glycosylation Pathways

### Abstract

Glycosylation plays important roles in cellular function and endows protein therapeutics with beneficial properties. However, constructing biosynthetic pathways to study and engineer precise glycan structures on proteins remains a bottleneck. Here we report a modular, versatile cell-free platform for glycosylation pathway assembly by rapid in vitro mixing and expression (GlycoPRIME). In GlycoPRIME, glycosylation pathways are assembled by mixing-and-matching cell-free synthesized glycosyltransferases that can elaborate a glucose primer installed onto protein targets by an N-glycosyltransferase. We demonstrate GlycoPRIME by constructing 37 putative protein glycosylation pathways, creating 23 unique glycan motifs, 18 of which have not yet been synthesized on proteins. We use selected pathways to synthesize a protein vaccine candidate with an α-galactose adjuvant motif in a one-pot cell-free system and human antibody constant regions with minimal sialic acid motifs in glycoengineered Escherichia coli. We anticipate that these methods and pathways will facilitate glycoscience and make possible new glycoengineering applications.

### A. Introduction

Protein glycosylation, the enzymatic process that attaches oligosaccharides to amino acid sidechains, is among the most abundant and complex post-translational modifications in nature^{1, 2} and plays critical roles in human health¹. Glycosylation is present in over 70% of protein therapeutics³ and profoundly affects protein stability^{4, 5}, immunogenicity^{6, 7}, and activity⁸. The importance of glycosylation in biology and evidence that intentional manipulation of glycan structures on proteins can improve therapeutic properties^{4, 6, 8} have motivated many efforts to study and engineer protein glycosylation structures⁹⁻¹¹.

Unfortunately, glycoprotein engineering is constrained by the number and diversity of glycan structures that can be built on proteins and platforms available for glycoprotein production^{9, 12}. A key challenge is that glycans are synthesized in nature by many glycosyltransferases (GTs) across several subcellular compartments 1, complicating engineering efforts and resulting in structural heterogeneity^{3, 12}. Furthermore, essential biosynthetic pathways in eukaryotic organisms limit the diversity of glycan structures that can be engineered in those systems^{9, 13}. Bacterial glycoengineering addresses these limitations by expressing heterologous glycosylation pathways in laboratory Escherichia coli strains that lack endogenous glycosylation enzymes^{13, 14}. Several asparagine (N-linked) glycosylation pathways have been successfully reconstituted in bacterial cells¹³⁻¹⁷ and cell-free systems¹⁸⁻²¹. In particular, cell-free systems, in which proteins and metabolites are synthesized in crude cell lysates, can accelerate the characterization and engineering of enzymes and biosynthetic pathways²²⁻²⁵. E. coli-based cell-free protein synthesis (CFPS) systems can produce gram per liter titers of complex proteins in hours,²⁶ enabling the rapid discovery, prototyping, and optimization of metabolic pathways without reengineering an organism for each pathway iteration²³⁻²⁵.

However, existing cell-free glycoprotein synthesis platforms have yet to fully exploit this paradigm because they rely on oligosaccharyltransferases (OSTs) to transfer prebuilt sugars from lipid-linked oligosaccharides (LLOs) onto proteins. OSTs are difficult to express because they are integral membrane proteins that often contain multiple subunitsl. Furthermore, the LLO substrate specificities of OSTs limit modularity and the diversity of glycan structures that can be transferred to proteins²⁷. Finally, LLOs competent for transfer by OSTs are difficult to synthesize in vitro¹². In fact, it has not yet been shown that LLO biosynthesis and glycosylation can be co-activated in vitro or that LLOs can be both transferred and extended in a bacterial CFPS system. Instead, LLOs must be derived from or pre-enriched in cell lysates by expression of LLO biosynthesis pathways in living cells¹⁸⁻²⁰. Expressing LLO biosynthesis pathways in cells requires time-consuming cloning and tuning of polycistronic operons, cellular transformation, and the production of new lysates for each glycan structure. Taken together, the complexity of membrane-associated OSTs and LLOs as well as OST substrate specificities present obstacles for glycoengineering and the facile construction and screening of multienzyme glycosylation pathways¹².

N-glycosyltransferases (NGTs) may overcome these limitations by enabling the construction of simplified, OST- and LLO-independent protein glycosylation pathways^{9, 16, 28}. NGTs are cytoplasmic, bacterial enzymes that transfer a glucose residue from a uracil-diphosphate-glucose (UDP-Glc) sugar donor onto asparagine sidechains²⁹. Importantly, NGTs are soluble enzymes that can install a glucose primer onto proteins in the E. coli cytoplasm^{16, 17, 22}. This primer can then be sequentially elaborated by co-expressed GTs^{16, 28}. Synthetic NGT-based glycosylation systems are not limited by OST substrate specificities and do not require protein transport across membranes or lipid-associated components⁹. These systems have elicited great interest as a complementary approach for synthesis of glycoproteins, including therapeutics and vaccines, that are difficult or impossible to produce using OST-based systems^{9, 16, 22, 28, 30-32}. Several recent advances set the stage for this vision. First, rigorous characterization of the acceptor specificity of NGTs using glycoproteomics and the GlycoSCORES technique^{17, 22, 31} have revealed that NGTs modify N-X-S/T amino acid motifs. Second, the NGT from Actinobacillus pleuropneumoniae (ApNGT) has been shown to modify native and rationally designed glycosylation sites within eukaryotic proteins in vitro and in E. coli^{16, 17, 22, 28}. Third, the Aebi group and others recently reported the elaboration of the glucose installed by ApNGT to polysialyllactose²⁸ or dextran¹⁶ motifs in E. coli cells as well as a chemoenzymatic method to transfer prebuilt oxazoline-functionalized oligosaccharides onto this glucose residue^{30, 32}. However, other biosynthetic pathways to build glycans using NGTs have not been explored⁹, perhaps due to slow timelines associated with building and testing synthetic glycosylation pathways in living cells. A cell-free synthesis platform based on ApNGT would accelerate glycoengineering efforts by enabling high-throughput and entirely in vitro construction, assembly, and screening of synthetic glycosylation pathways.

Here, we describe a modular, cell-free method for glycosylation pathway assembly by rapid in vitro mixing and expression (GlycoPRIME). In this two-pot method, crude E. coli lysates are selectively enriched with individual GTs by CFPS expression and then combined in a mix-and-match fashion to construct multienzyme glycosylation pathways. The goal of GlycoPRIME is to design, build, test, and analyze many combinations of enzymes without making new genetic constructs, strains, cell lysates, or purified enzymes for each combination to discover new biosynthetic pathways (including many not found in nature) to glycoprotein structures of interest. These enzyme combinations can then be transferred to biomanufacturing systems, such as living cells, and used to produce and test glycoproteins. A key feature of GlycoPRIME is the use of ApNGT to site-specifically install a single N-linked glucose primer onto proteins, which can be elaborated to a diverse repertoire of glycans. The use of ApNGT as the initiating glycosylation enzyme removes constraints on glycan structure imposed by OST specificities for LLOs and enables the first entirely in vitro glycosylation pathway synthesis and screening workflow by obviating the need to synthesize glycans on LLO precursors in living cells.

To validate GlycoPRIME, we optimize the in vitro expression of 24 bacterial and eukaryotic GTs and combine them to create 37 putative biosynthetic pathways to elaborate the glucose installed by ApNGT on a model glycoprotein substrate. We generated 23 unique glycan structures composed of 1 to 5 core saccharides and longer repeating structures. These pathways yielded 18 glycan structures that have not yet been reported on proteins and provide new biosynthetic routes to therapeutically relevant motifs including an α1-3-linked galactose (αGal) epitope as well as fucosylated and sialylated lactose or poly-N-acetyllactosamine (LacNAc). We then demonstrate that pathways identified using GlycoPRIME can be transferred to cell-free and cellular biosynthesis systems by producing (i) a protein vaccine candidate with an adjuvanting αGal glycan6, 7, 33 in a one-pot cell-free protein synthesis driven glycoprotein synthesis (CFPS-GpS) platform and (ii) the constant region (Fc) of the human immunoglobulin (IgG1) antibody in the E. coli cytoplasm with minimal sialic acid glycans known to improve in vivo pharmacokinetics^{5, 34}. The GlycoPRIME method represents a powerful new approach to accelerate the construction and screening of multienzyme glycosylation pathways. By identifying feasible synthetic glycosylation pathways, we anticipate that GlycoPRIME will enable future efforts to produce and engineer glycoproteins for compelling applications including fundamental studies and improved therapeutics.

### B. Establishing an In Vitro Glycoengineering Platform

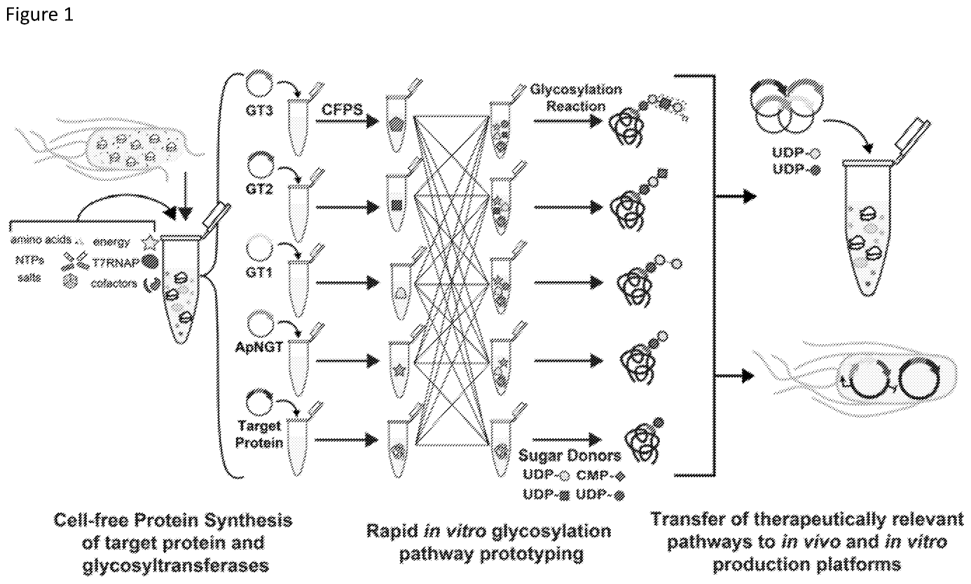

We established GlycoPRIME as a modular, in vitro protein synthesis and glycosylation platform to develop biosynthetic pathways which elaborate the N-linked glucose priming residue installed by ApNGT to diverse glycosylation motifs including sialylated and fucosylated forms of lactose and LacNAc as well as an αGal epitope (FIG. 1).

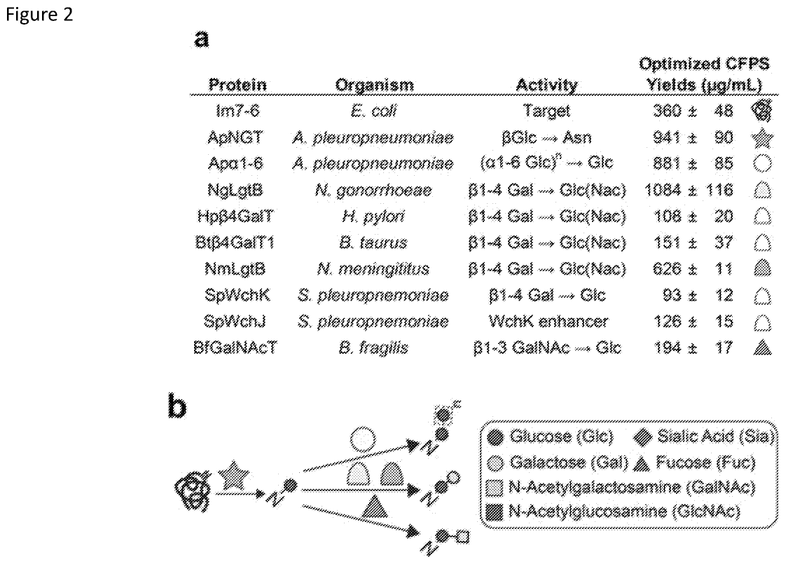

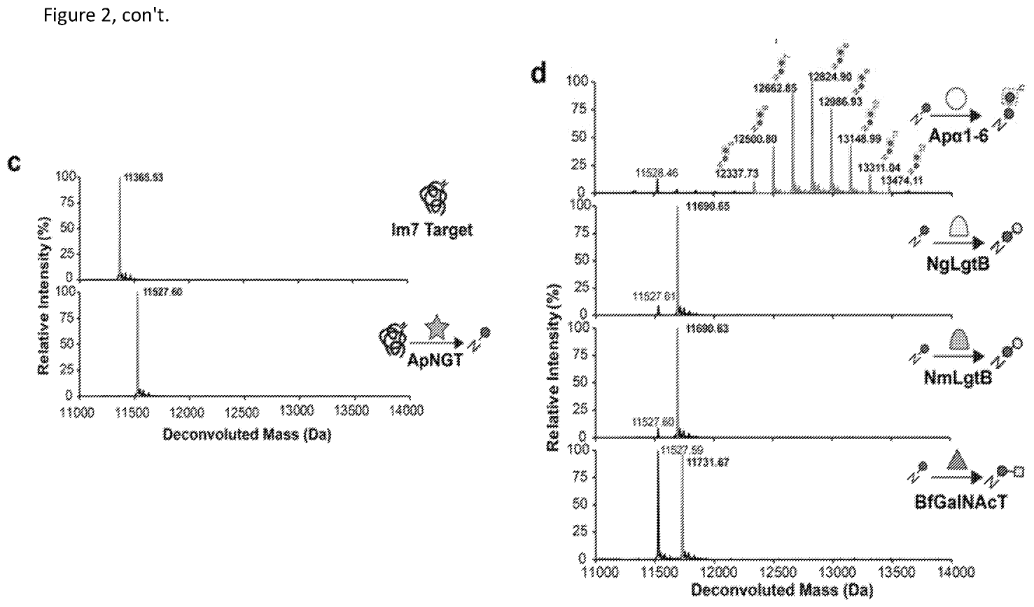

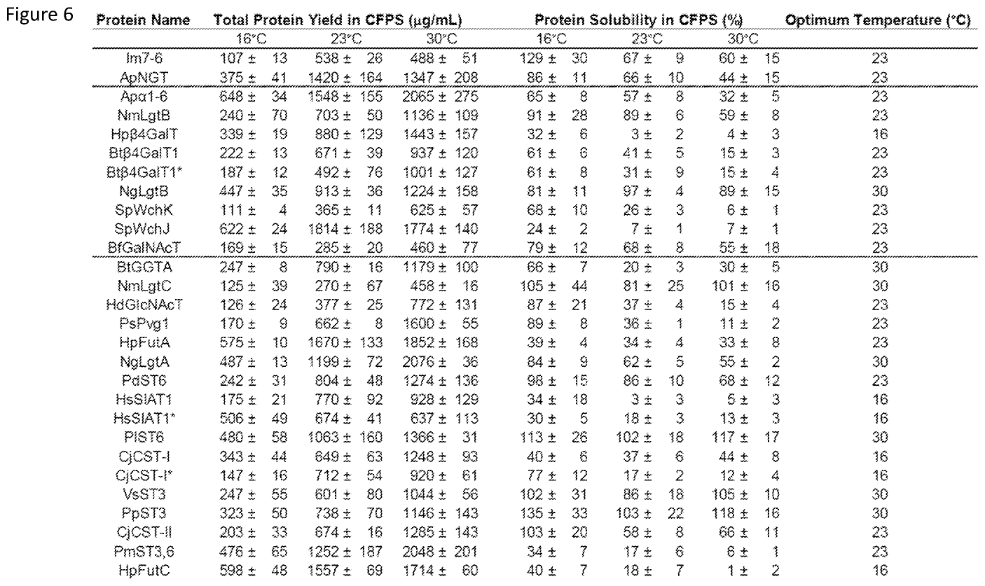

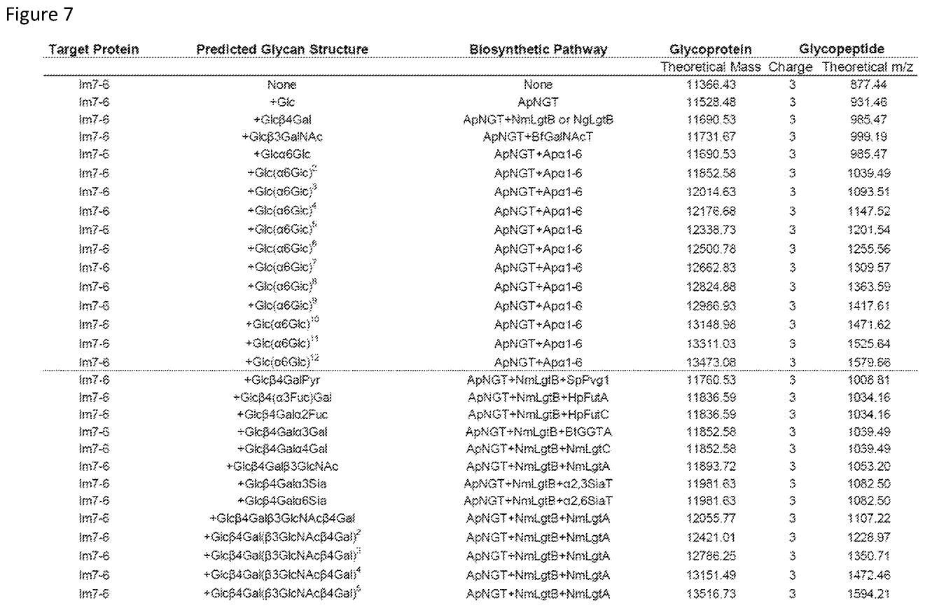

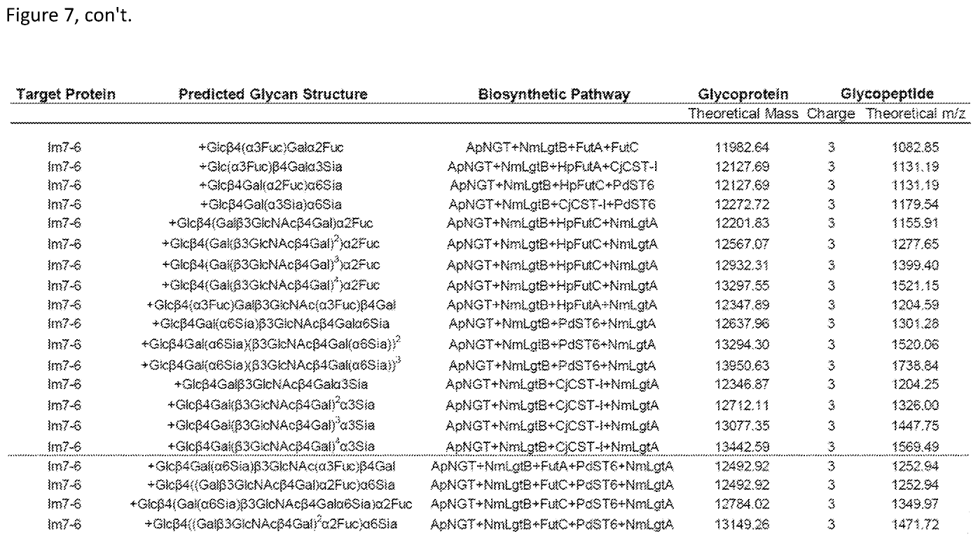

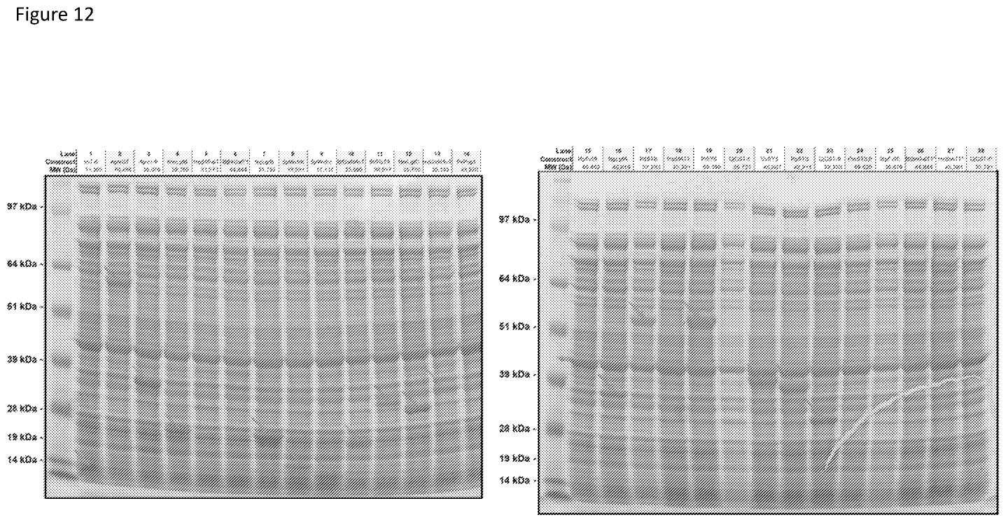

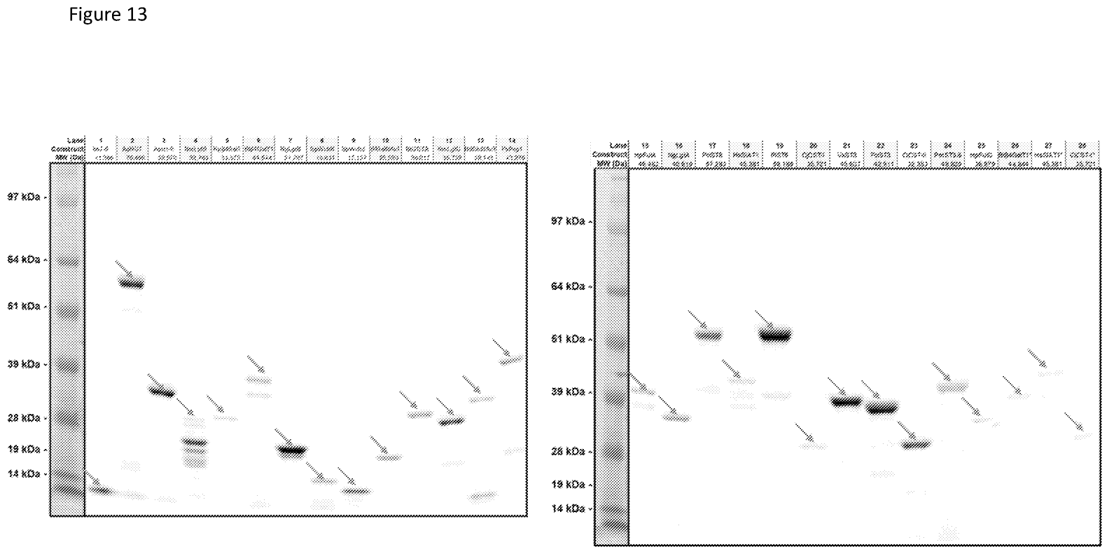

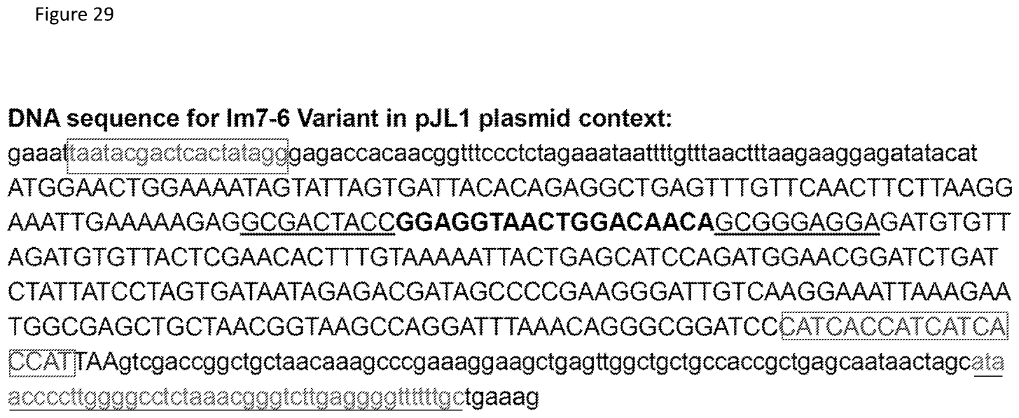

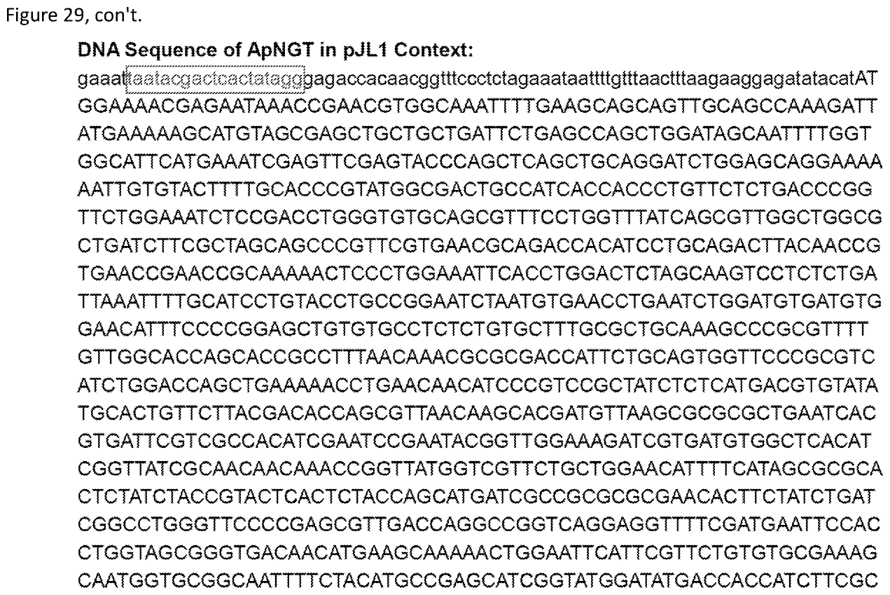

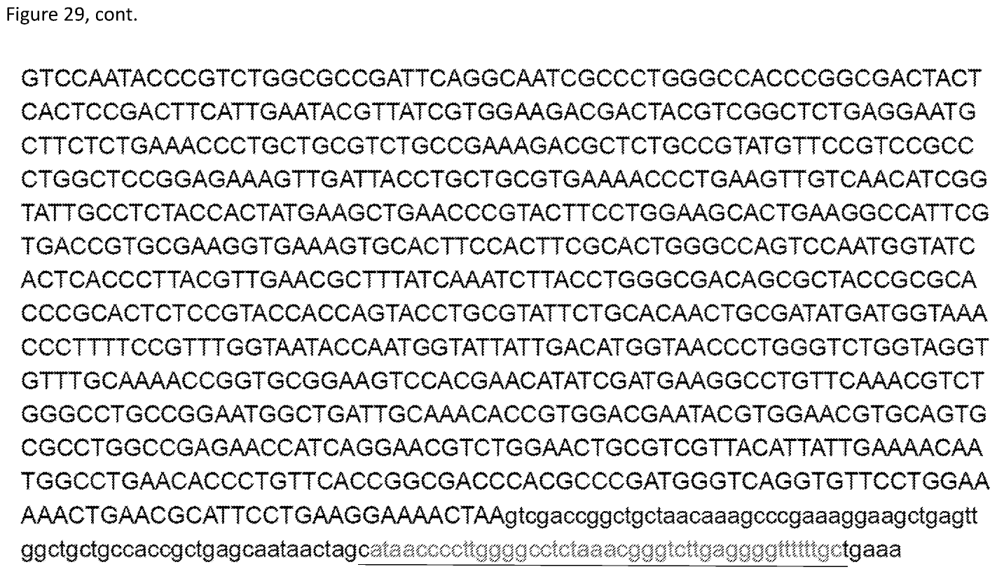

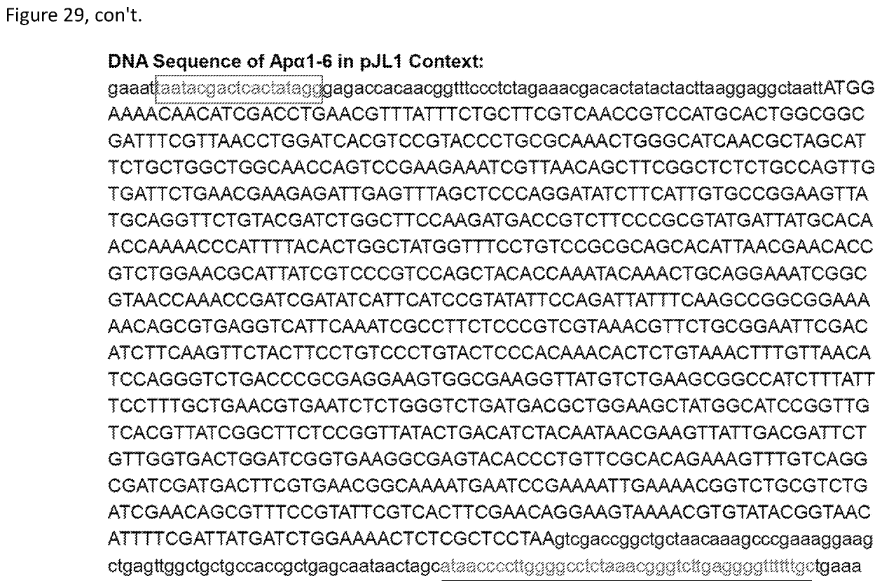

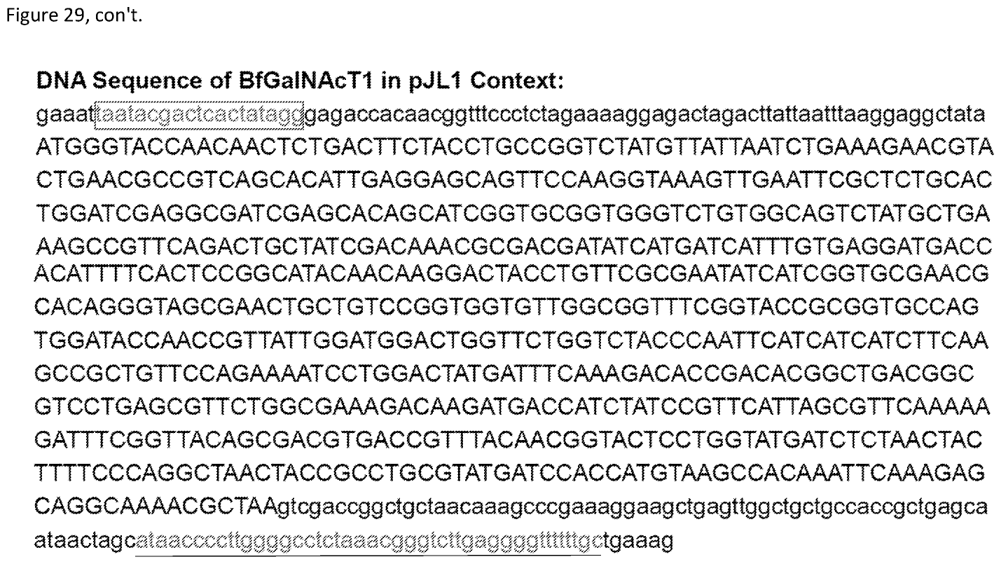

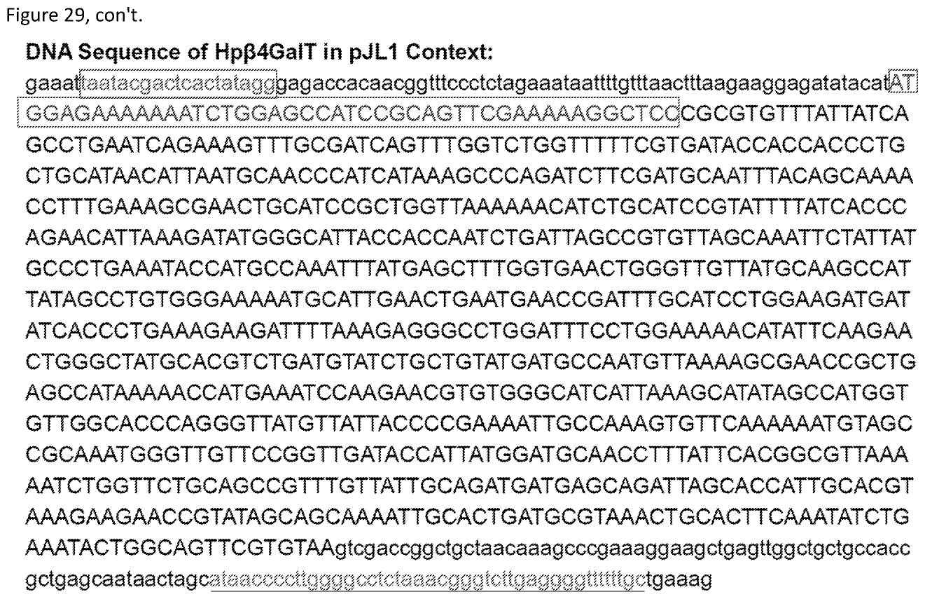

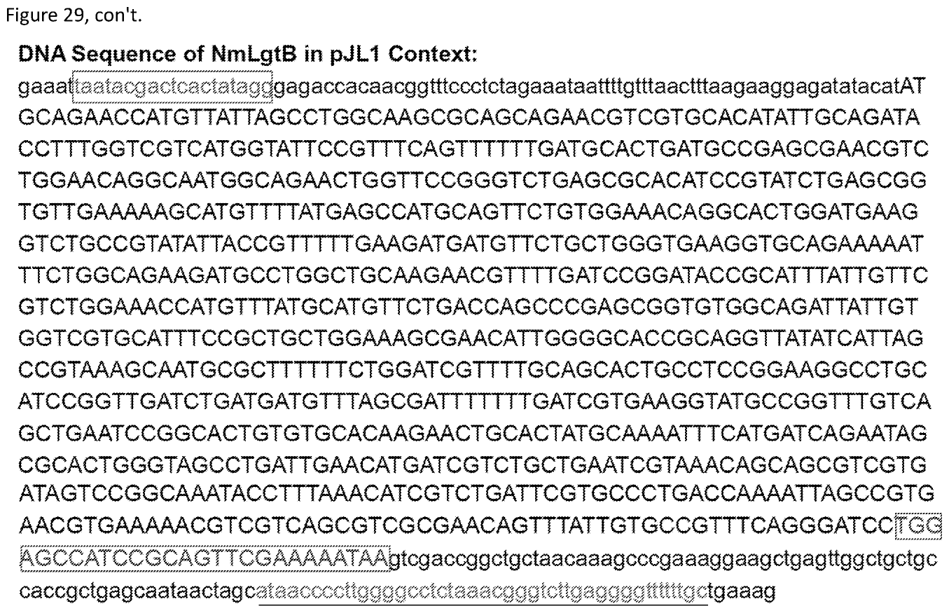

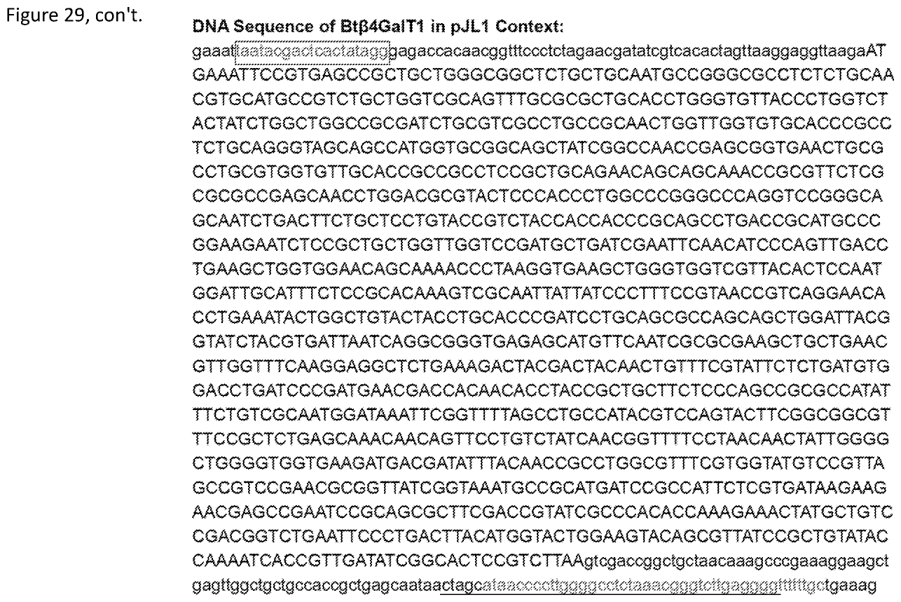

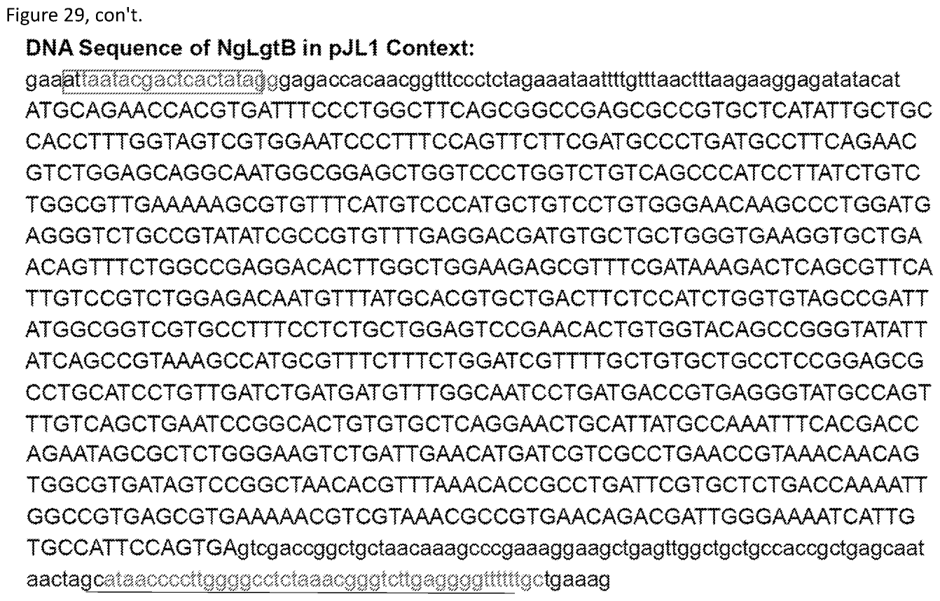

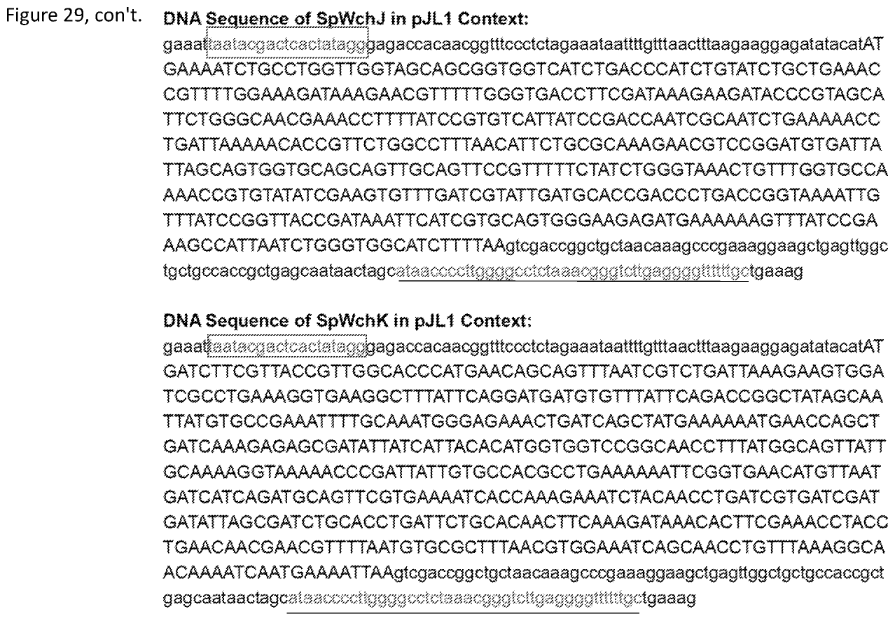

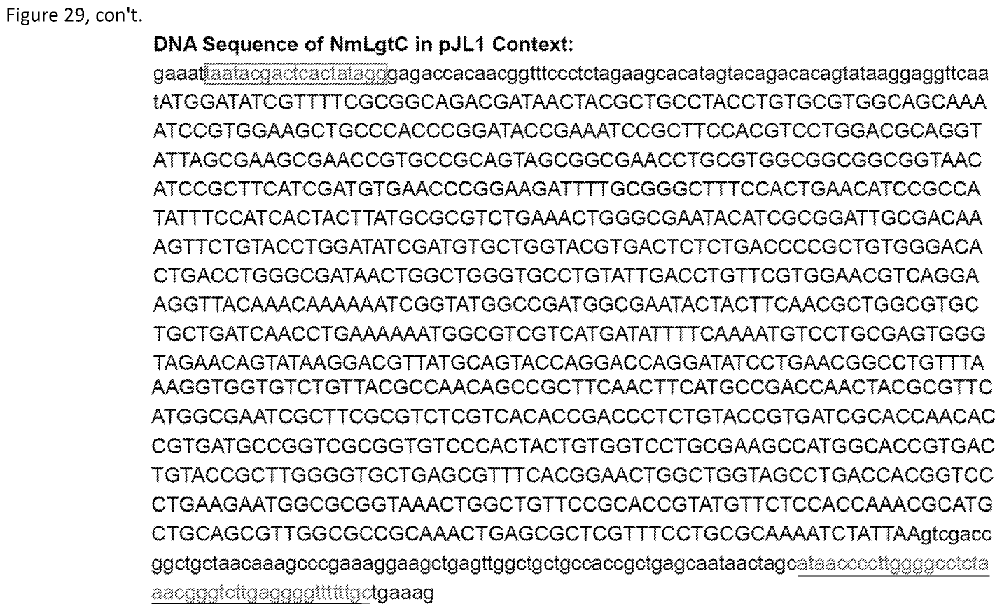

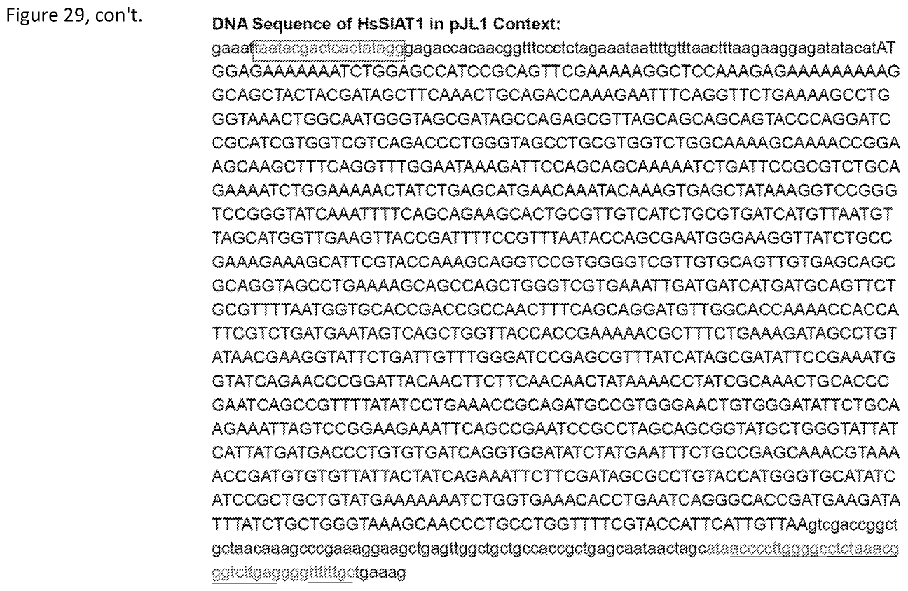

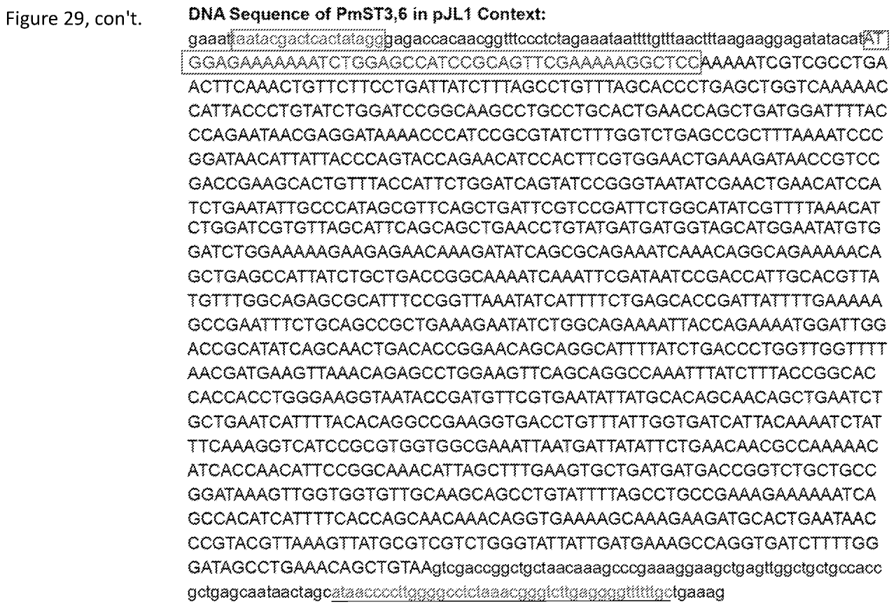

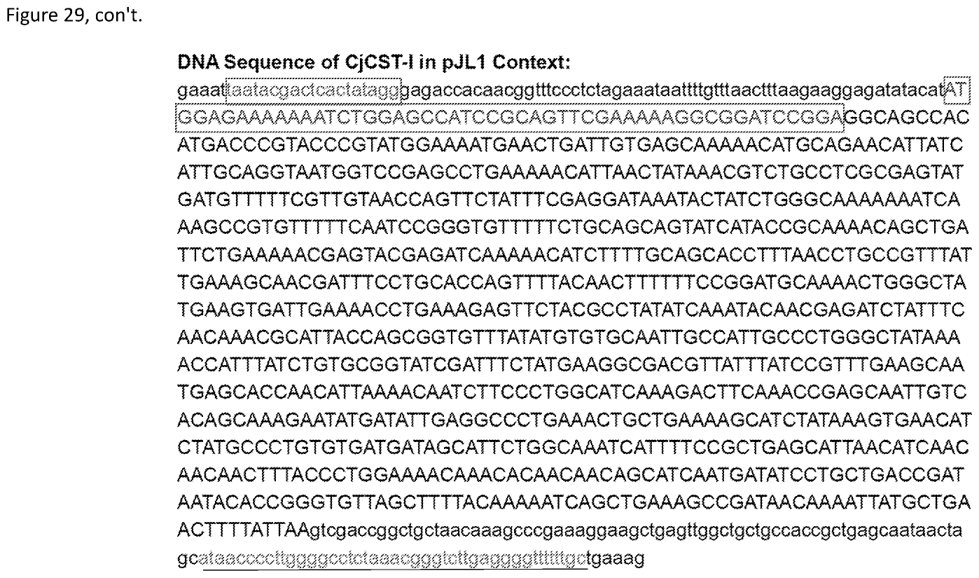

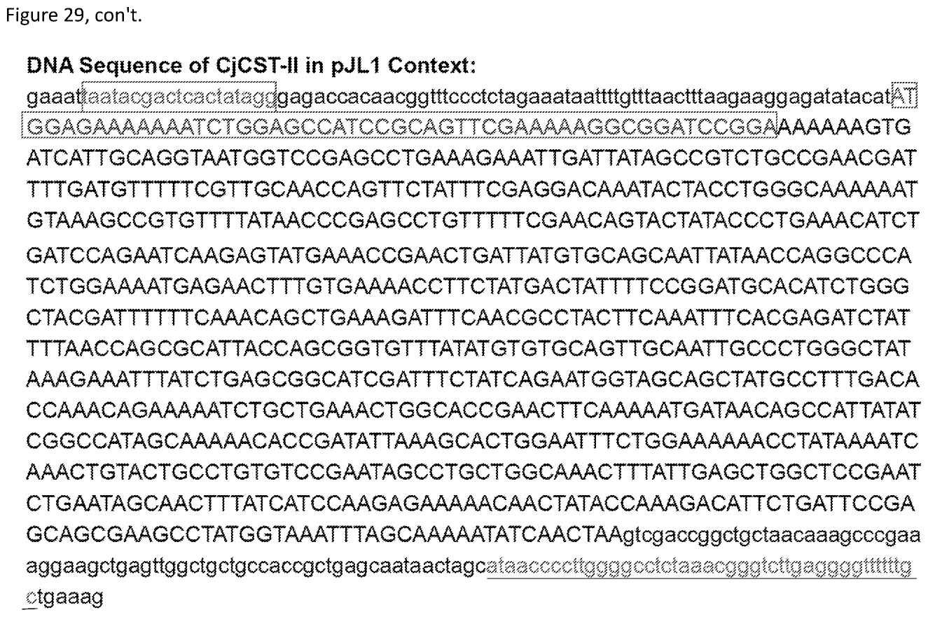

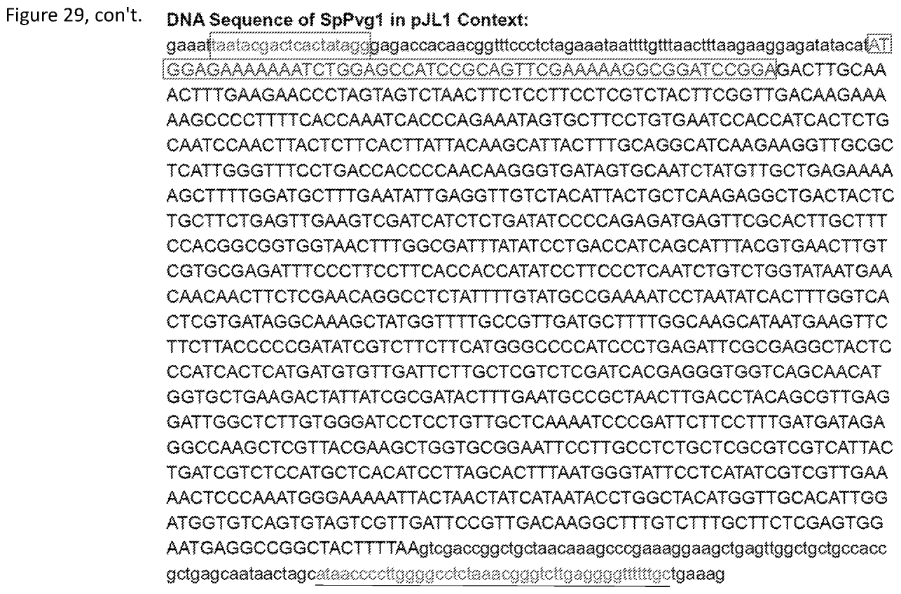

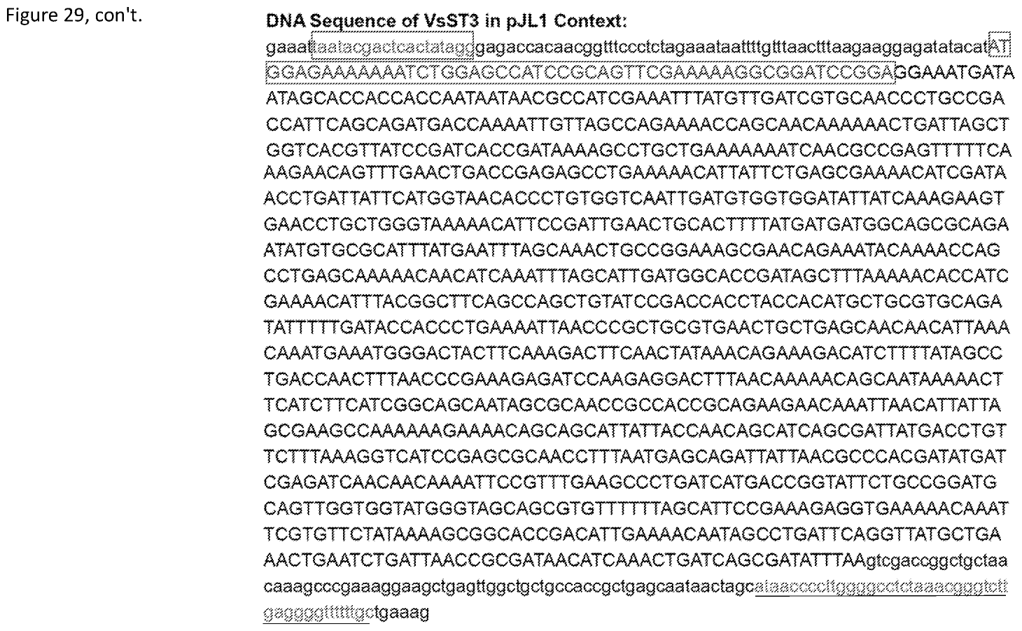

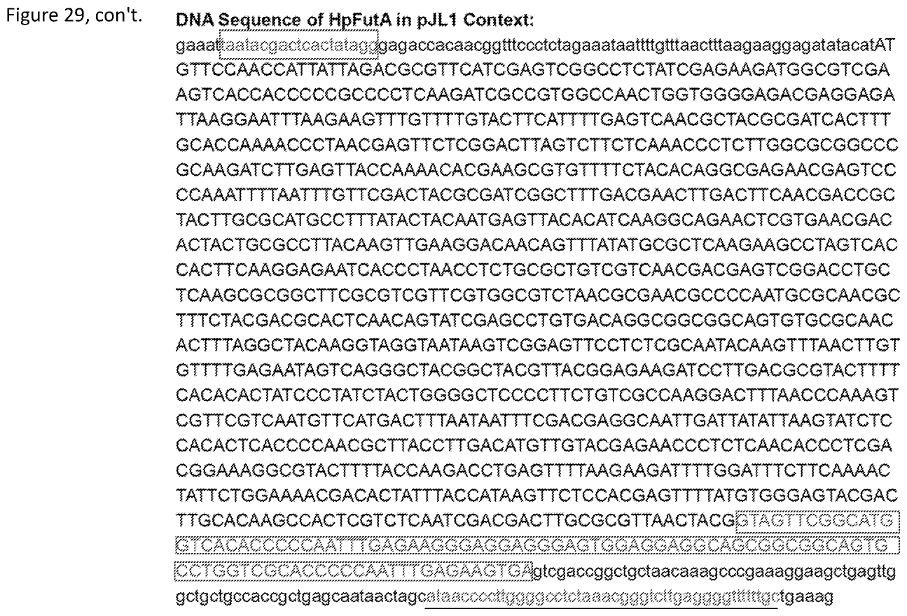

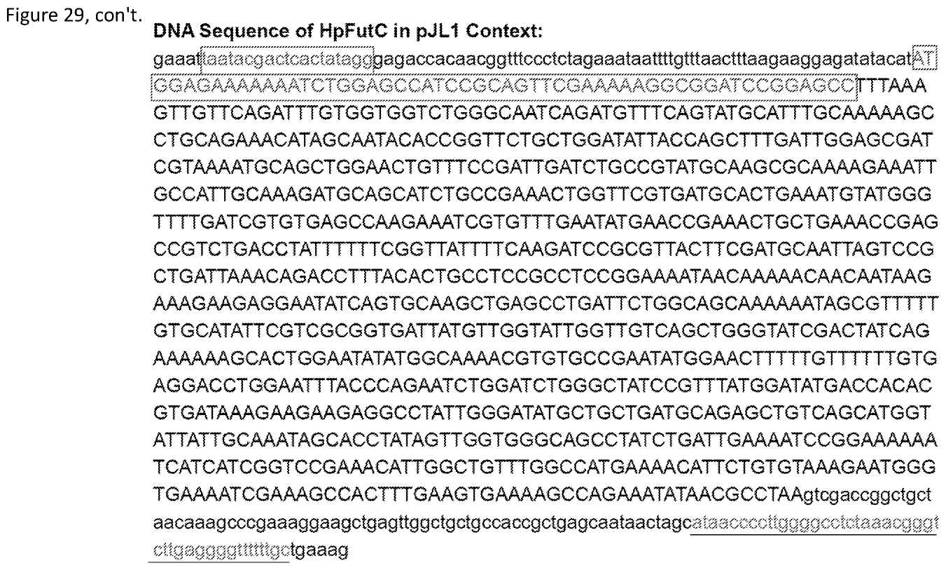

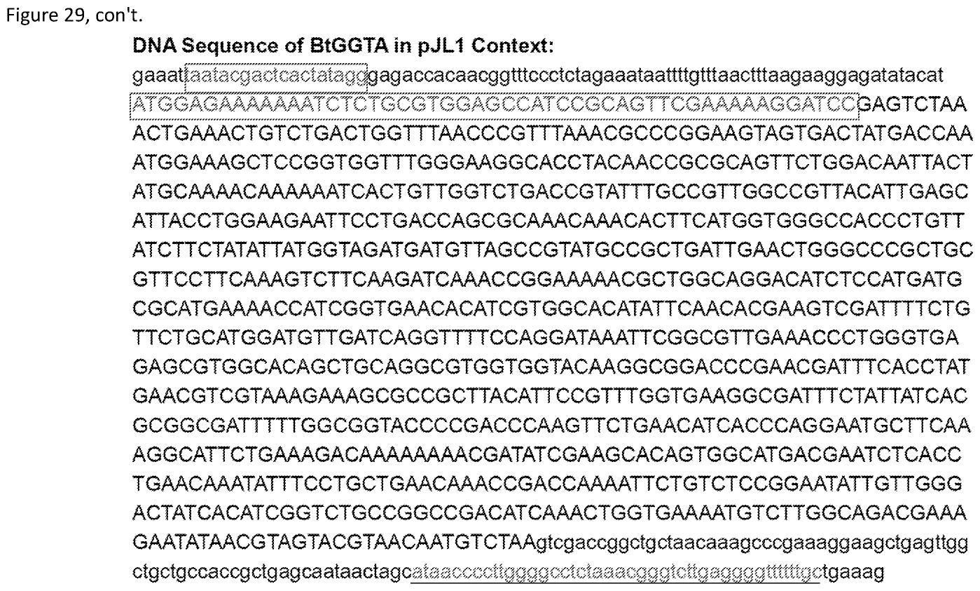

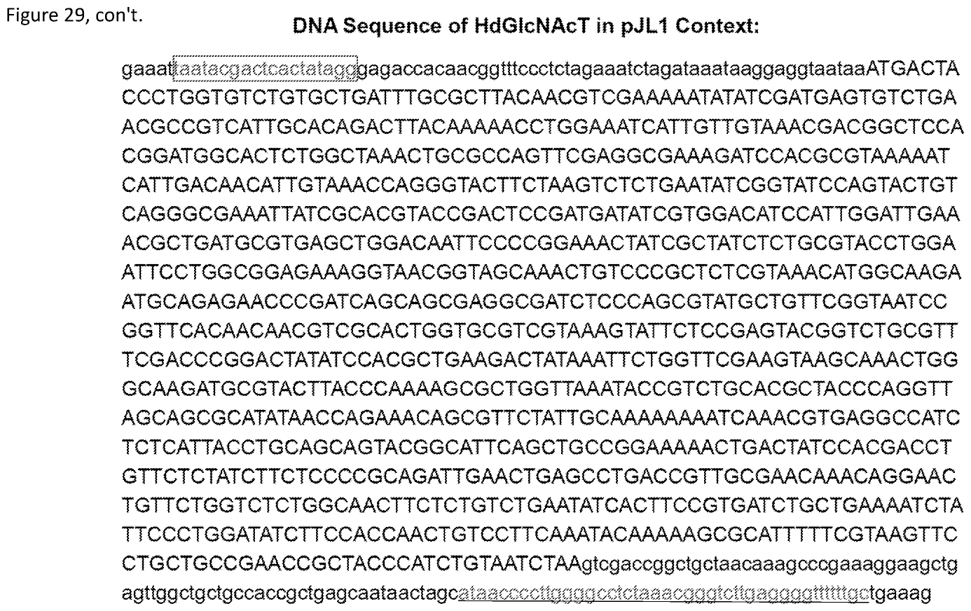

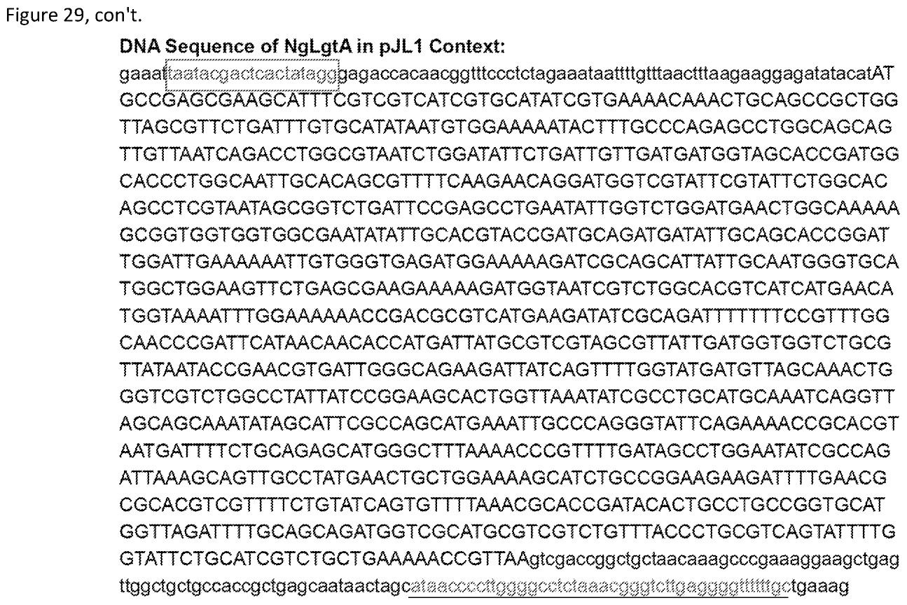

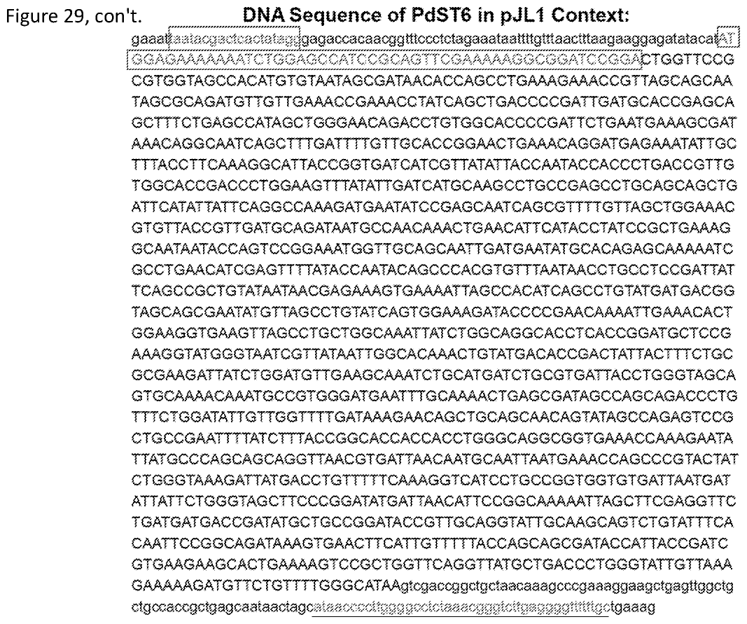

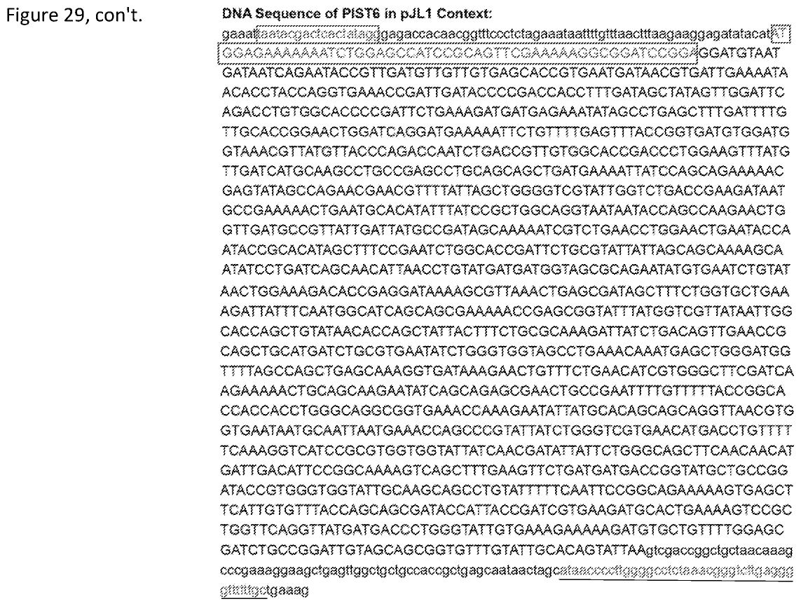

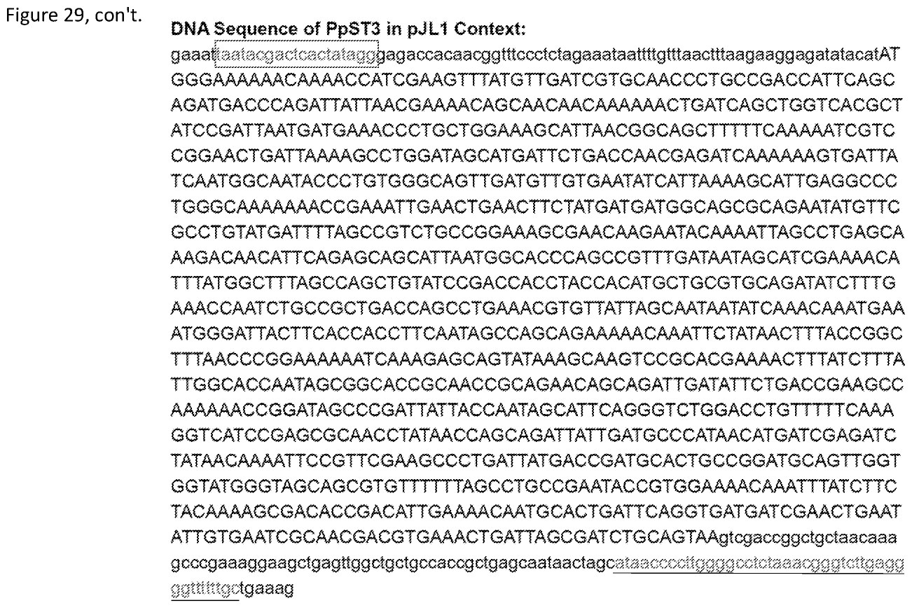

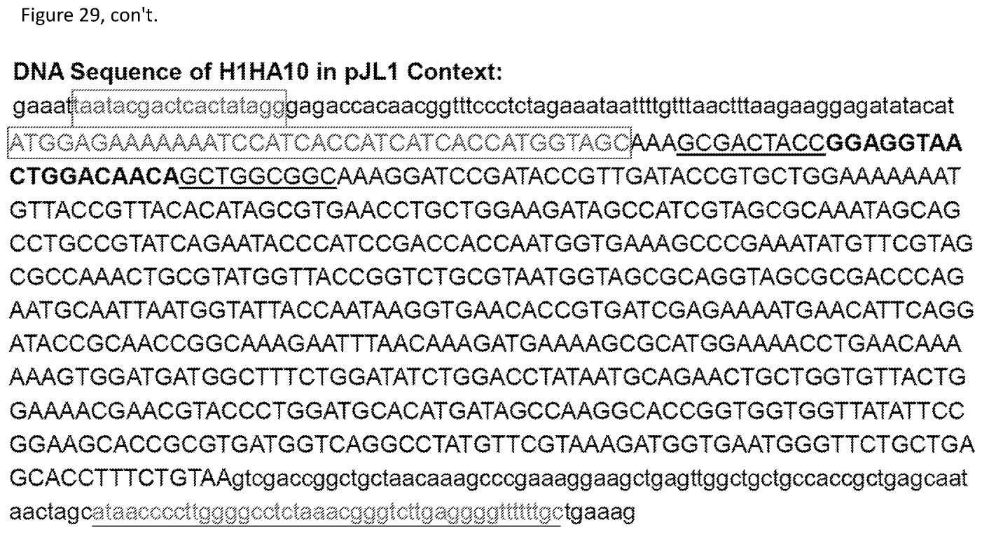

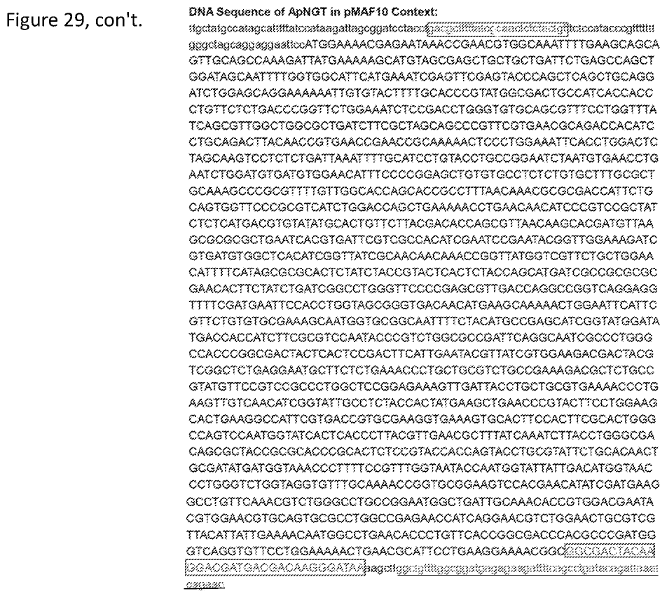

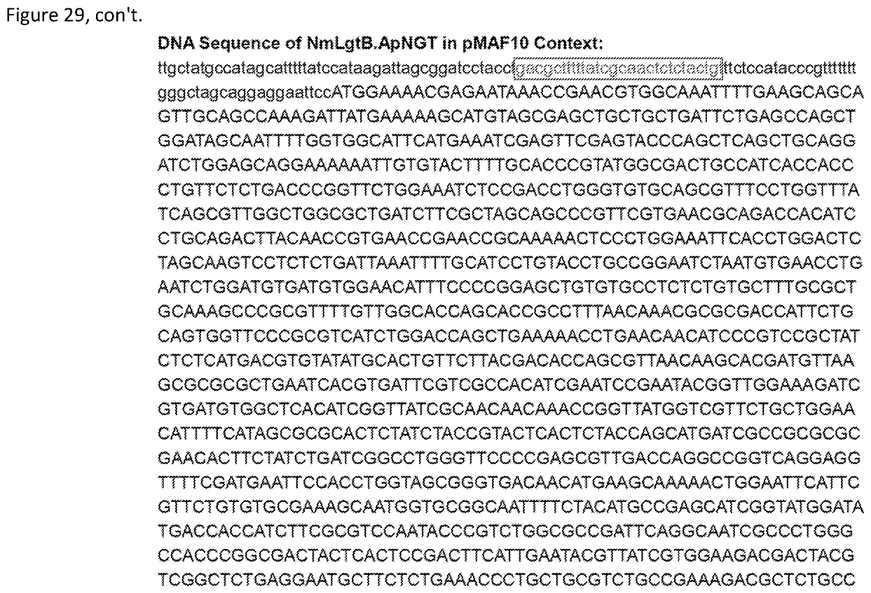

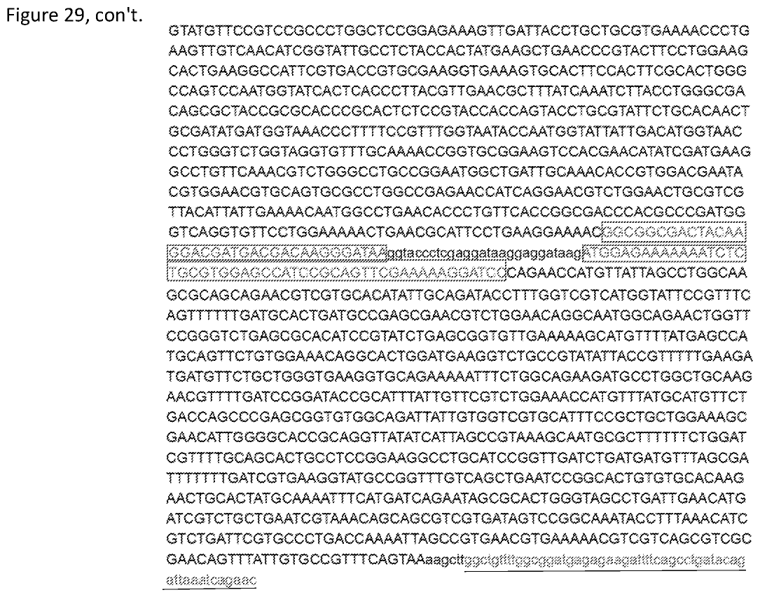

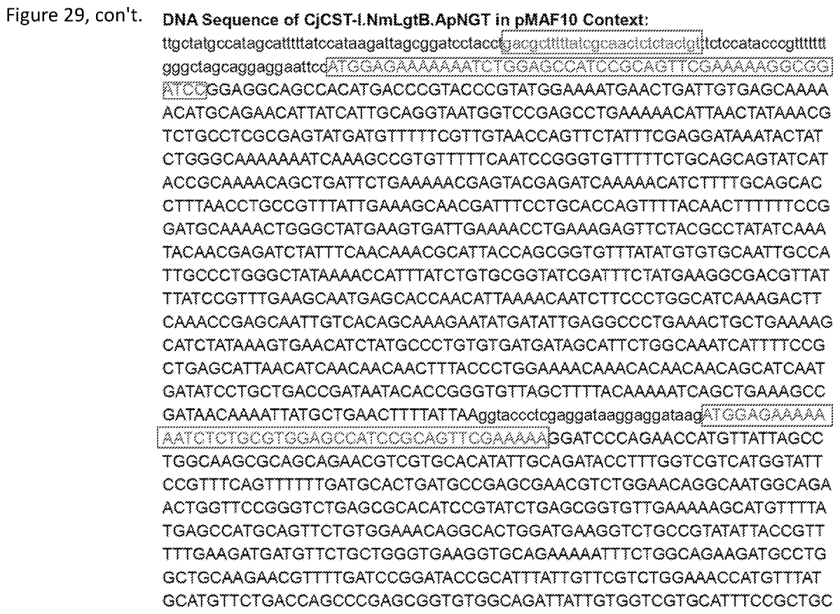

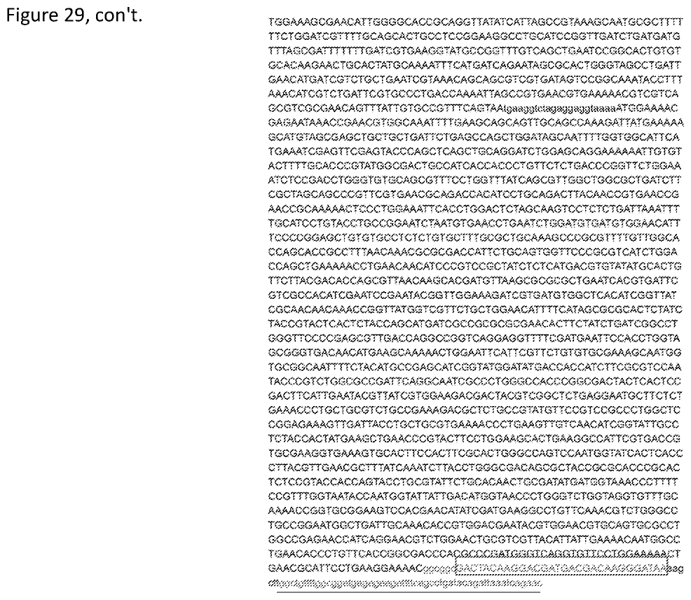

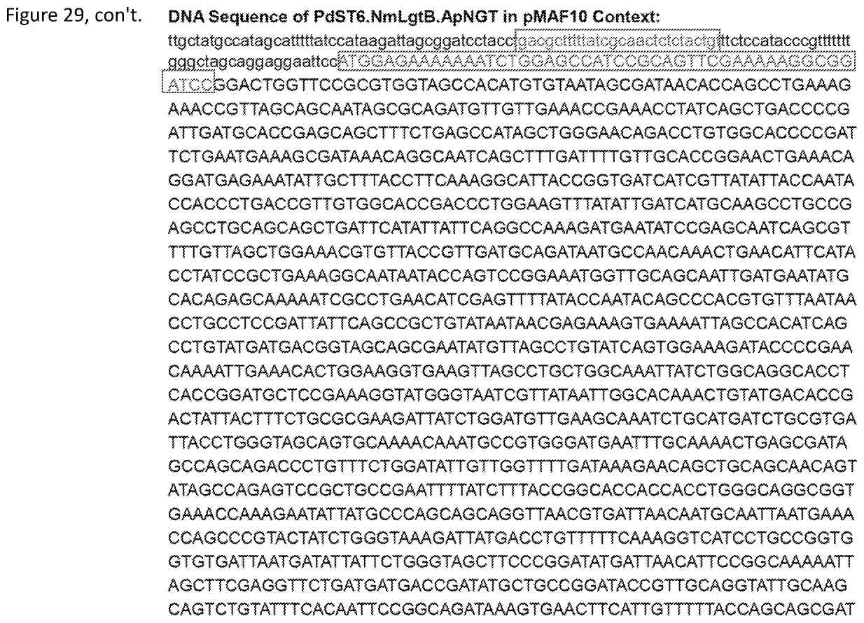

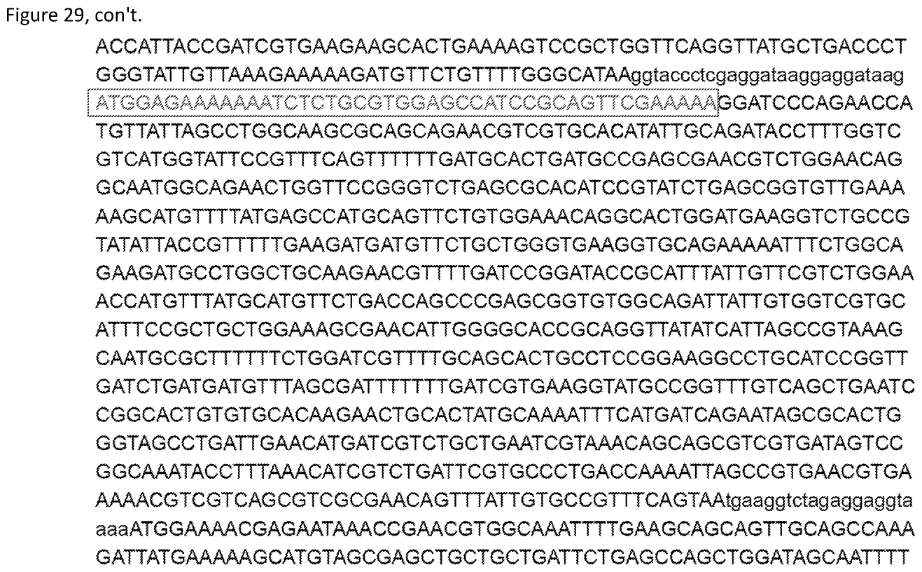

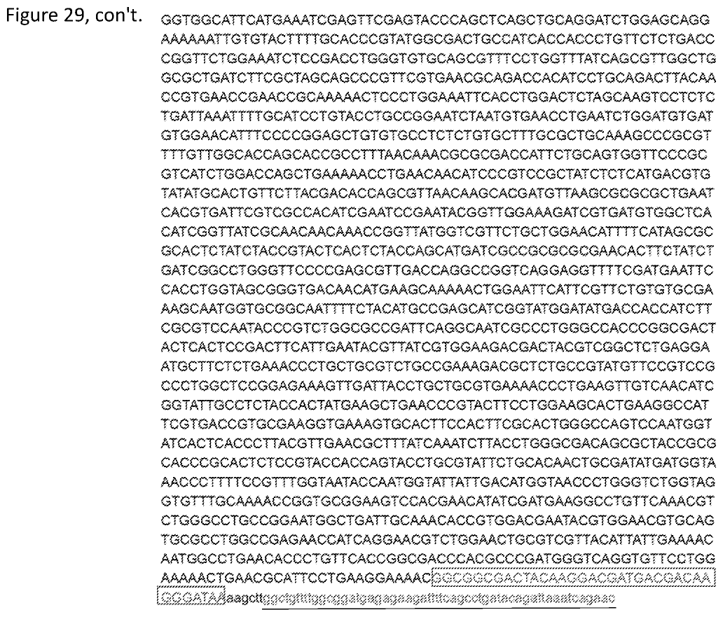

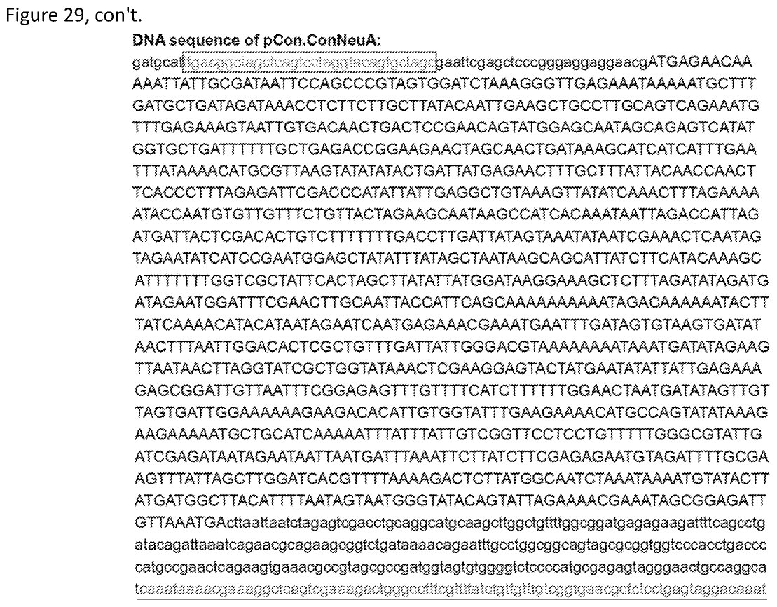

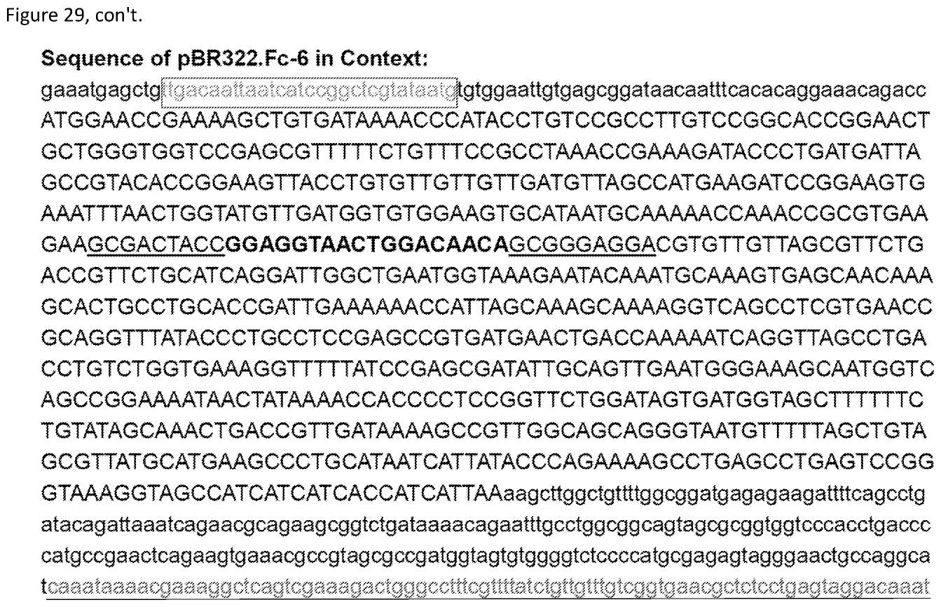

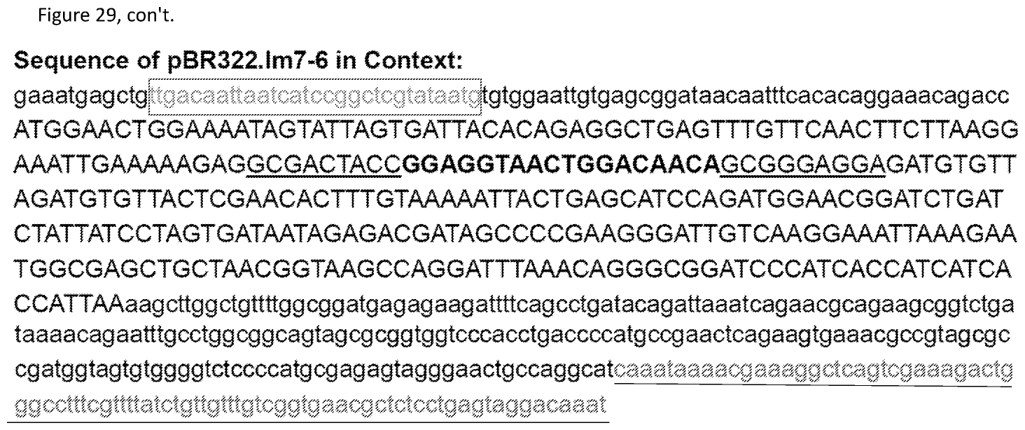

For proof of concept, we aimed to glycosylate a model protein with ApNGT in a setting that would enable further glycan elaboration in our GlycoPRIME workflow. Specifically, we identified CFPS conditions that provided high GT expression titers so that the minimum volume of GT-enriched lysate required for complete glycoprotein conversion could be added to each in vitro glycosylation (IVG) reaction, leaving sufficient reaction volume and generating the substrate for further elaboration by mixing cell-free lysates. Based on our previous characterization of ApNGT acceptor sequence specificity²², we selected an engineered version of the E. coli immunity protein Im7 (Im7-6) bearing a single, optimized glycosylation sequence of GGNWTT at an internal loop as our model target protein (FIG. 5 and FIG. 29). We used [14C]-leucine incorporation to measure and optimize the CFPS reaction temperature for our engineered Im7-6 target and ApNGT (FIG. 6 and FIG. 2a) and confirmed their full-length expression by SDS-PAGE autoradiogram (FIGS. 12 and 13). We found that 23° C. provided the most soluble product for these proteins, balancing greater overall protein production at higher temperatures and greater solubility at lower temperatures. We synthesized Im7-6 and ApNGT by CFPS and then mixed those reaction products together along with UDP-Glc in a 32-μl IVG reaction. We then purified the Im7-6 substrate using Ni-NTA functionalized magnetic beads and performed intact glycoprotein liquid chromatography mass spectrometry (LC-MS) (see Methods). We observed nearly complete conversion of 10 μM of Im7-6 substrate (11 μl) with just 0.4 μM ApNGT (1 μl) (FIG. 2c), as indicated by a mass shift of 162 Da (the mass of a glucose residue) in the deconvoluted protein mass spectra (theoretical masses shown in FIG. 7). This shows that CFPS products can be directly assembled into IVG reactions to produce glycoprotein with remaining reaction volume for the addition of elaborating GTs.

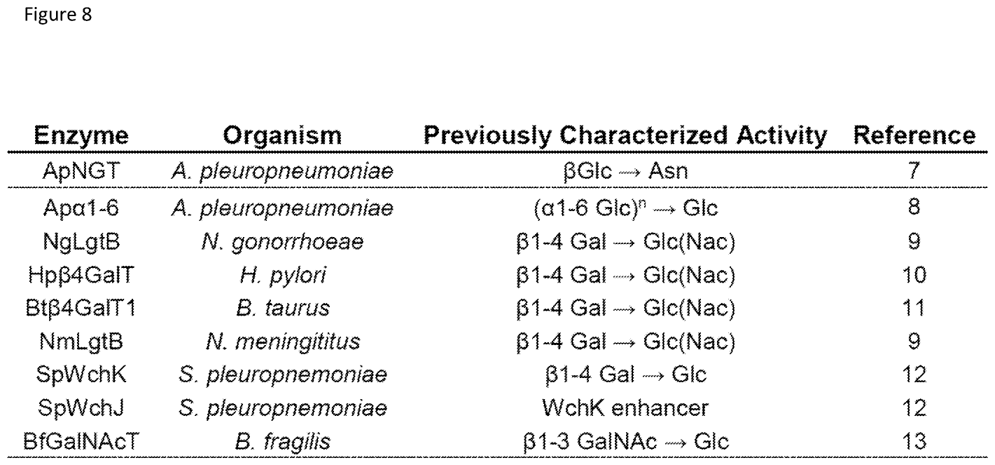

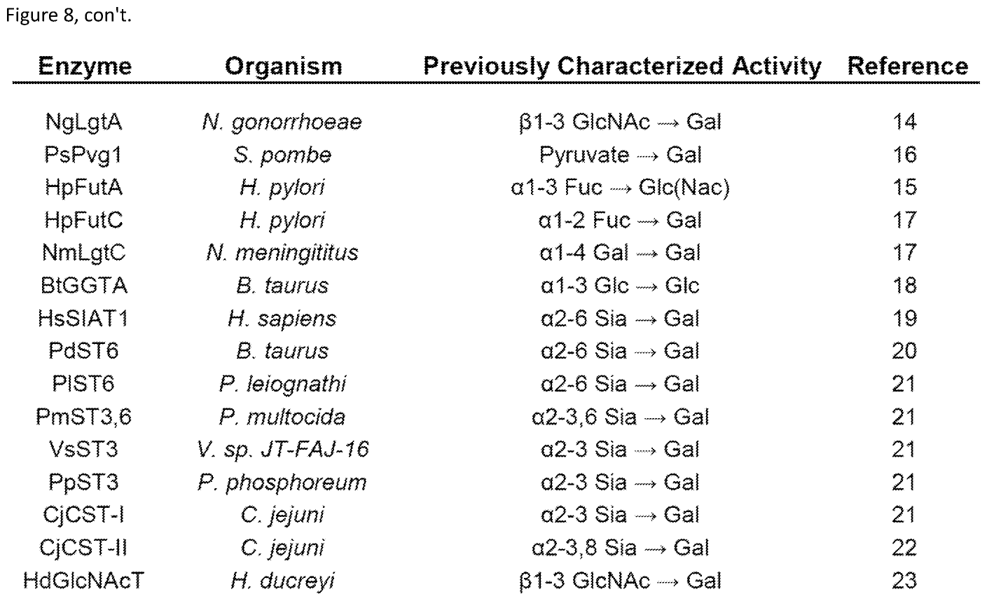

Next, we identified 7 GTs with previously characterized specificities that could be useful in elaborating the glucose primer installed by ApNGT to relevant glycans (FIG. 2 and FIG. 8). Previous works indicate that in A. pleuropnemoniae, the glucose installed by ApNGT is modified by the polymerizing Apα1-6 glucosyltransferase to form N-linked dextran29 and that this structure could be a useful vaccine antigenl6, 35. Recent work also showed that the β1-4 galactosyltransferase LgtB from Neisseria meningitis (NmLgtB) can modify an ApNGT-installed glucose in E. coli, forming N-linked lactose (Asn-Glcβ1-4Gal)28. Here, we attempted to recapitulate these pathways in vitro and selected 5 additional enzymes with potentially useful activities (FIG. 2a). We chose the N-acetylgalactosamine (GalNAc) transferase from Bacteroides fragilis (BfGalNAcT) because the GalNAc residue it insta11s36 could serve as an elaboration point for O-linked glycan epitopes. We also chose several β1-4 galactosyltransferases from Streptococcus pneumoniae (SpWchK), Neisseria gonorrhoeae (NgLgtB), Helicobacter pylori (Hpβ4GalT), and Bos taurus (Btβ4GalT1) to determine the optimal biosynthetic route to N-linked lactose. This was important because lactose is a known substrate of many GTs that modify milk oligosaccharides and the termini of human N-linked glycans1, 37-40, making it a critical reaction node for further glycan diversification.

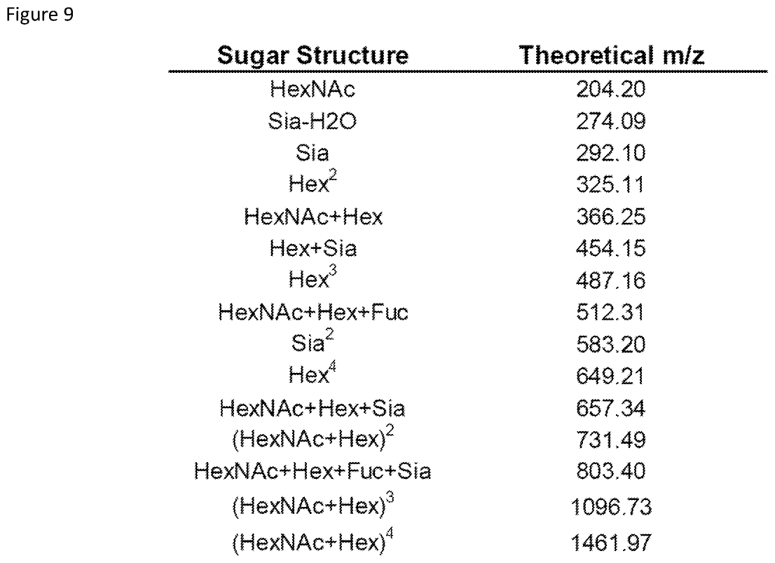

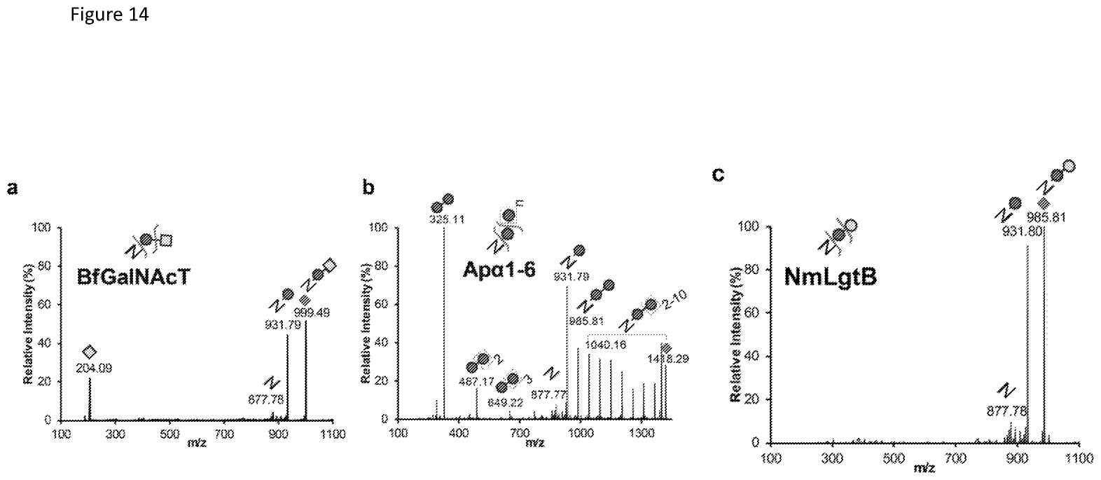

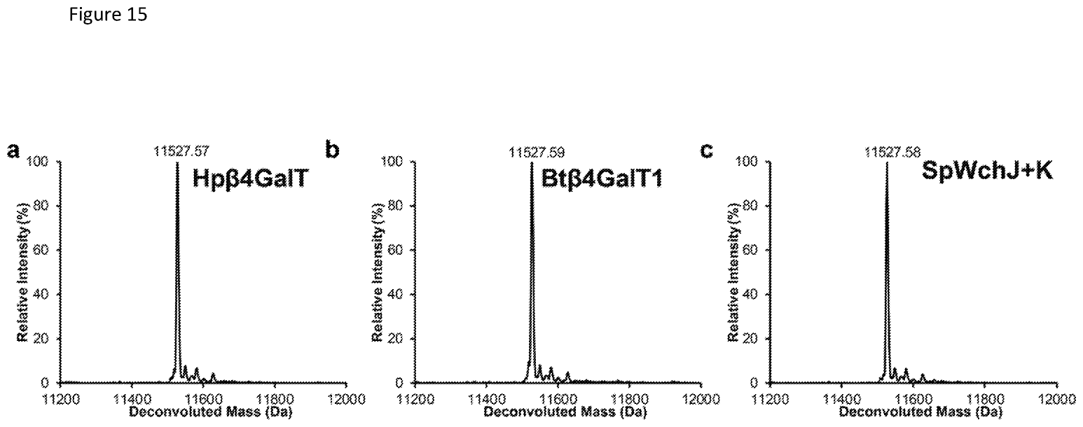

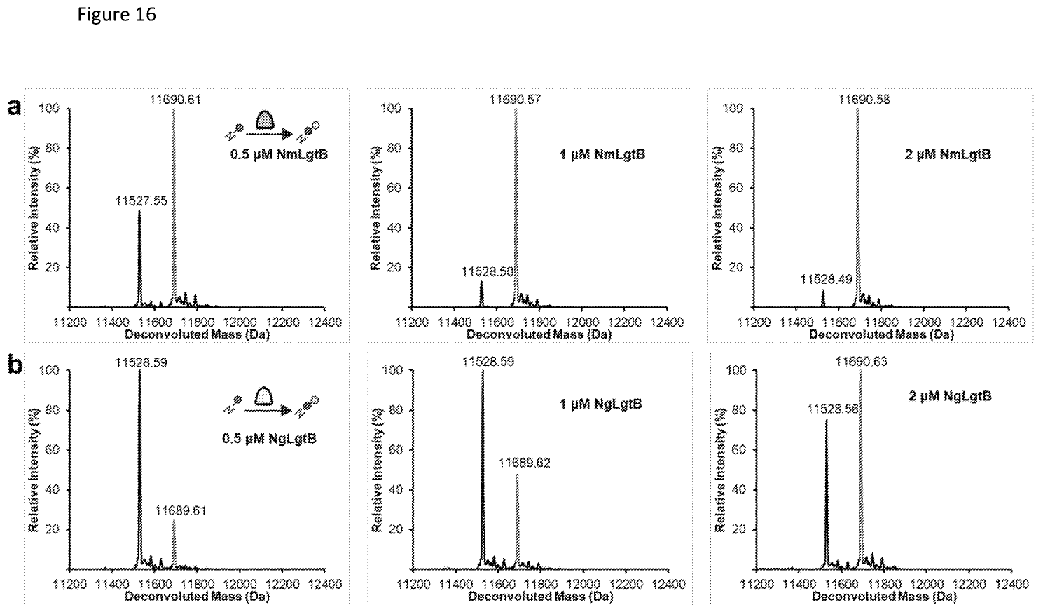

Once identified, we optimized CFPS conditions and confirmed the soluble, full-length expression of these 7 GTs (FIG. 2, FIG. 6, and FIGS. 12 and 13), as well as SpWchJ from S. pneumoniae, which is known to enhance the activity of SpWchK41. We then assembled IVG reactions by mixing CFPS products containing these GTs with Im7-6 and ApNGT CFPS products along with UDP-Glc and other appropriate sugar donors according to previously characterized activities (FIG. 2). We observed Im7-6 intact mass shifts and tandem MS (MS/MS) fragmentation spectra of trypsinized glycopeptides consistent with the known activities of NmLgtB and NgLgtB (β1-4 galactosyltransferases), BfGalNAcT (a β1-3 N-acetylgalactosyltransferase), and Apα1-6 (a polymerizing α1-6 glucosyltransferase) (FIG. 2, FIG. 14, and FIG. 9). We did not observe modification by Hpβ4GalT, SpWchK (even with SpWchJ), or Btβ4GalT1 (even with α-lactalbumin and conditions conducive to disulfide bond formation) (FIG. 15). By testing IVGs with decreasing amounts of NmLgtB and NgLgtB, we found that 2 μM of NmLgtB provided nearly complete conversion to N-linked lactose whereas the same amount of NgLgtB was less efficient (FIG. 16). These results show that multienzyme glycosylation pathways can be rapidly synthesized, combinatorially assembled, and evaluated in vitro. Using this approach, we found that ApNGT and NmLgtB provide an efficient in vitro route to N-linked lactose and discovered that ApNGT and BfGalNAcT can site-specifically install a GalNAc-terminated glycan.

### C. Modular Construction of Diverse Glycosylation Pathways

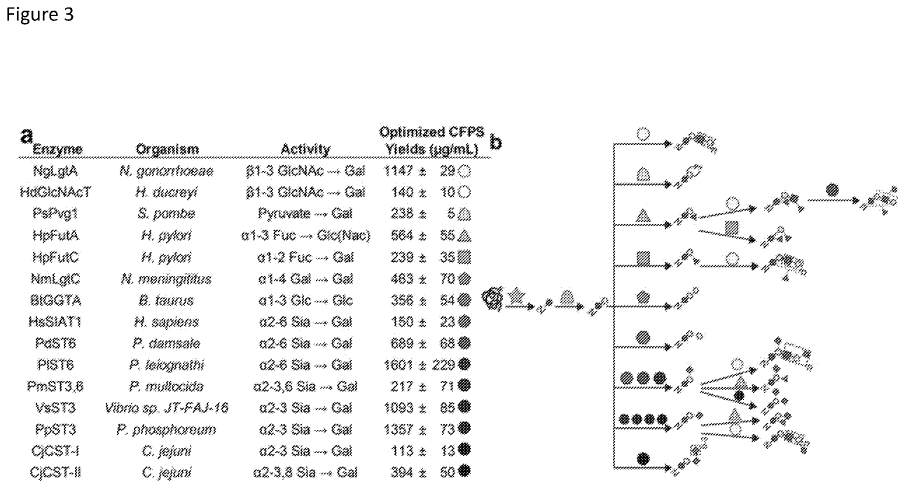

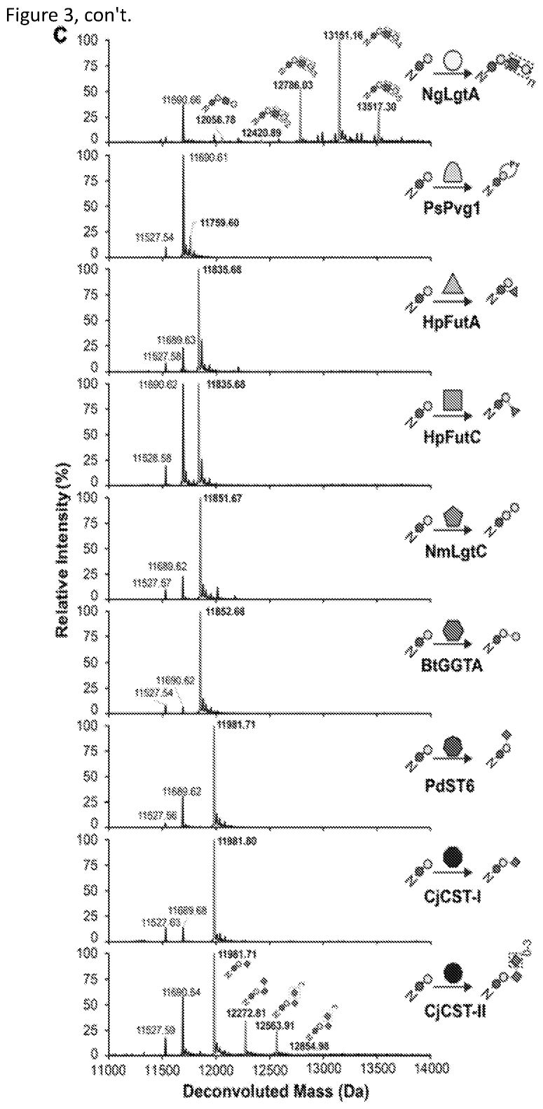

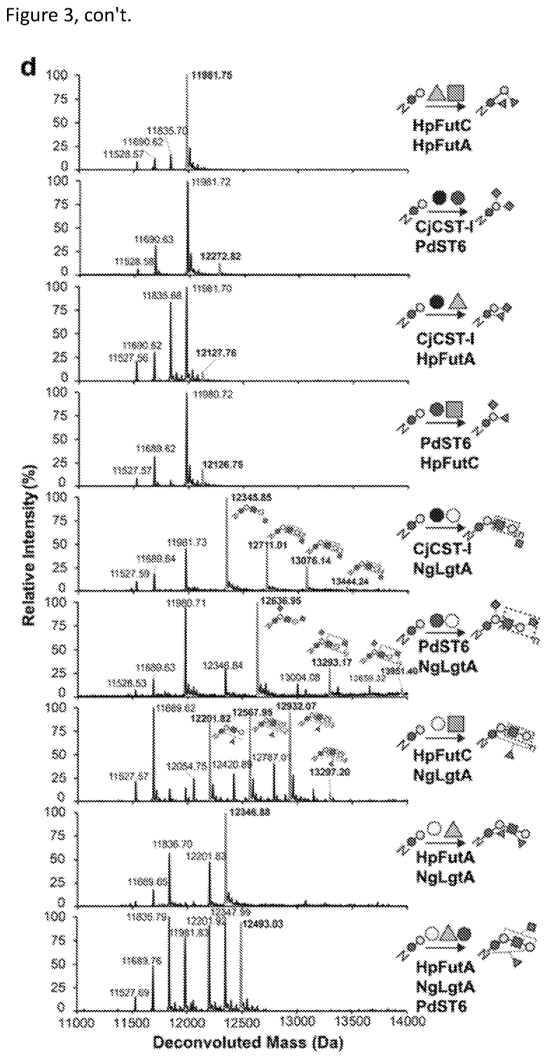

To demonstrate the power of GlycoPRIME for modular pathway construction and screening, we next selected 15 GTs with known specificities that suggested their ability to elaborate the N-linked lactose installed by ApNGT and NmLgtB into a diverse repertoire of 3 to 5 saccharide motifs and longer repeating structures (FIG. 3 and FIG. 8). Specifically, we sought to discover biosynthetic pathways that elaborate N-linked lactose to 9 oligosaccharides containing sialic acid (Sia), galactose (Gal), pyruvate, fucose (Fuc), and LacNAc. From there, we could obtain even greater diversity by recombining these GTs in various ways. We first describe our rationale for selecting these pathway classes, including their potential value for a variety of applications, and then present our experimental results.

Our first aim was to build glycans terminated in sialic acids because they provide many useful properties for applications in protein therapeutics^{5, 8, 28, 34, 42} (such as improved trafficking, stability, and pharmacodynamics); functional biomaterial⁴³; binding interactions with bacterial receptors^{44, 45,} human galectins⁴⁶, and siglecs⁴⁷; as well as adjuvants⁴⁸ and tumor-associated carbohydrate antigens (TACAs) for vaccines^{49, 50}. As the linkages of terminal sialic acids are important for these applications, we selected enzymes to install Sia with α2-3, α2-6, and α2-8 linkages onto the N-linked lactose. We began by building a 3′-sialyllactose (Glcβ1-4Galα2-6Sia) structure which could provide several useful properties including specific binding to pathogen receptors that adhere to human cells⁴⁴, delivery of vaccines to macrophages for increased antigen presentation⁴⁴, and mimicry of the human GM3 ganglioside (ceramide-Glcβ1-4Galα2-3Sia) for cancer vaccines⁵⁰. The 3′-sialyllactose structure may also mimic the recently reported GlycoDelete structure (GlcNAcβ1-4Galα2-3Sia), a simplified N-glycan known to preserve glycoprotein therapeutic activity and pharmacokinetics⁵¹. To build 3′-sialyllactose, we chose four α2-3 sialyltransferases from Pasteurella multocida (PmST3,6), Vibrio sp JT-FAJ-16 (VsST3), Photobacterium phosphoreum (PpST3), and Campylobacter jejuni (CjCST-I). Next, we aimed to discover biosynthetic routes to 6′-sialyllactose (Glcβ1-4Galα2-6Sia) because N-glycans bearing terminal α2-6Sia are common in secreted human proteins⁵, exhibit anti-inflammatory properties8, enable targeting of B cells for treatment of lymphoma⁵², and provide a distinct set of siglec, lectin, and receptor binding profiles^{5, 44, 47}. To produce 6′-sialyllactose, we selected three α2-6 sialyltranferases from humans (HsSIAT1), Photobacterium damselae (PdST6), and Photobacterium leiognathid (P1ST6). Finally, we investigated pathways to produce glycans with α2-8Sia that may mimic the GD3 ganglioside (ceramide-Glcβ1-4Galα2-3Siaα2-8Sia), a TACA and possible vaccine epitope against melanoma^{5, 44, 47}. Based on previous works²⁸′ ^{42,} we selected the CST-II bifunctional sialyltranferase from C. jejuni to install terminal α2-8Sia. In addition to Sia-containing glycans, we explored the synthesis of pyruvalated galactose because this structure displays similar lectin-binding properties to Sia⁵⁴. To build terminally pyruvylated lactose, we selected a pyruvyltransferase from Schizosaccharomyces pombe (SpPvgl)⁵⁴.

Beyond structures terminated in Sia, we explored pathways to modify N-linked lactose with Gal, Fuc, and LacNAc. For example, we aimed to engineer a first-of-its-kind bacterial system for complete biosynthesis of proteins modified with αGal (Glcβ1-4Galα1-3Gal) epitopes. αGal is an effective self:non-self discrimination epitope in humans and is bound by an estimated 1% of the human IgG pool^{6, 7, 33}. Consequently, αGal confers adjuvant properties when associated with various peptide, protein, whole-cell, and nanoparticle-based immunogens6, 7, 33, 55. To build αGal, we selected the α1,3 galactosyltransferase from B. taurus (BtGGTA). In addition, we sought to synthesize the globobiose structure (Glcβ1-4Galα1-4Gal) because it may mimic the Gb3 ganglioside (ceramide-Glcβ1-4Galα1-4Gal) which can bind and neutralize Shiga-like toxins secreted by pathogenic bacteria⁵⁶. We selected the galactosyltransferase LgtC from N. meningitis (NmLgtC) to synthesize globobiose. We also aimed to build LacNAc because it provides useful properties for biomaterials⁵⁷ as well as the inhibition and modulation of galectins to control cancer, inflammation, and fibrosis⁵⁸. We selected two β1-3 N-acetylglucosamine (GlcNAc) transferases from N. gonorrhoeae (NgLgtA) and Haemophilus ducreyi (HdGlcNAcT) to make this structure. Finally, we aimed to build fucosylated lactose structures which may find applications in biomaterials for neuronal tissue⁵⁹ as well as targeting or preventing the adherence of bacteria⁶⁰. To synthesize fucosylated lactose, we screened α1,3 and α1,2 fucosyltransferases from H. pylori (HpFutA and HpFutC, respectively).

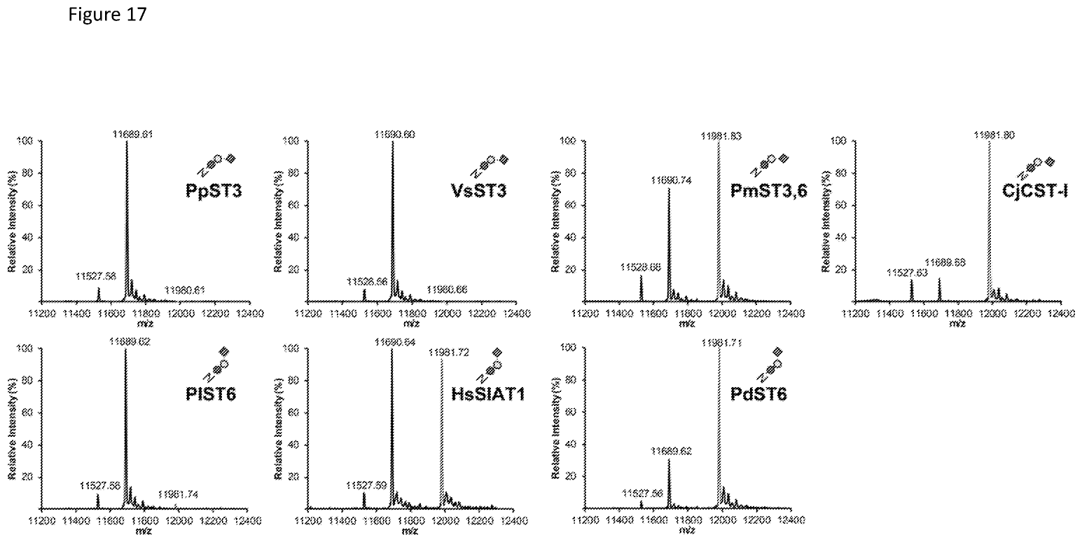

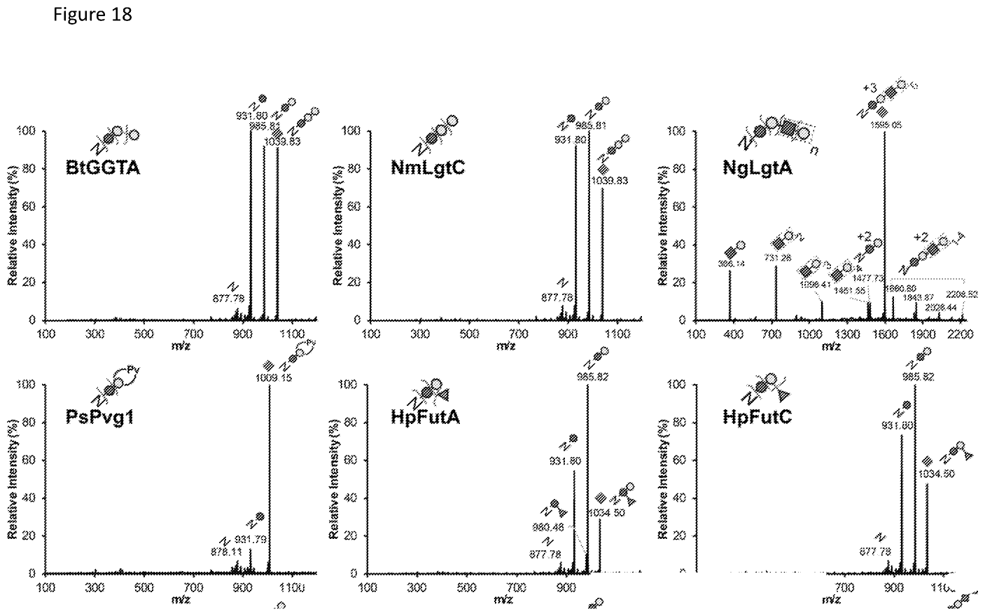

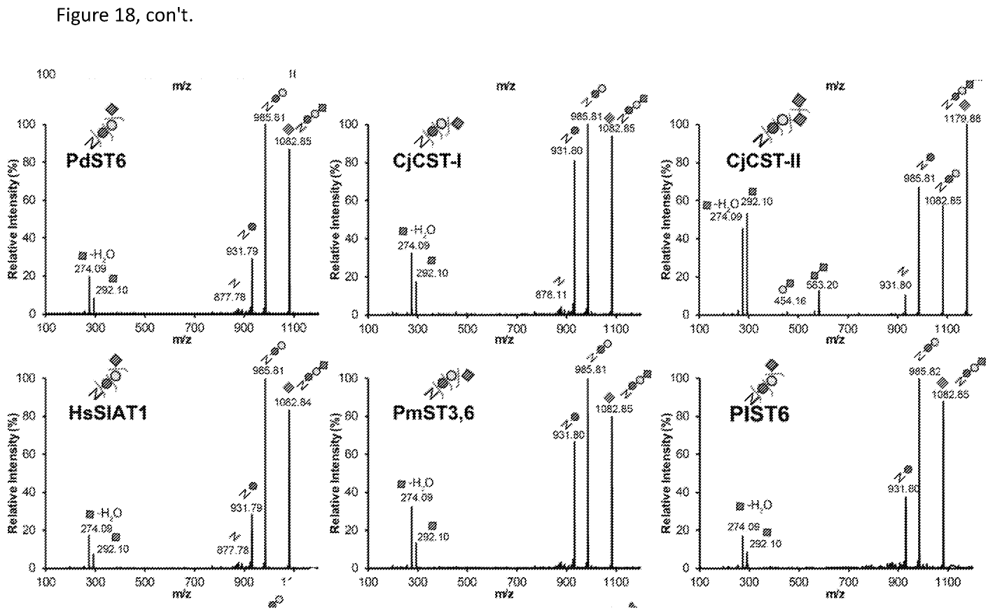

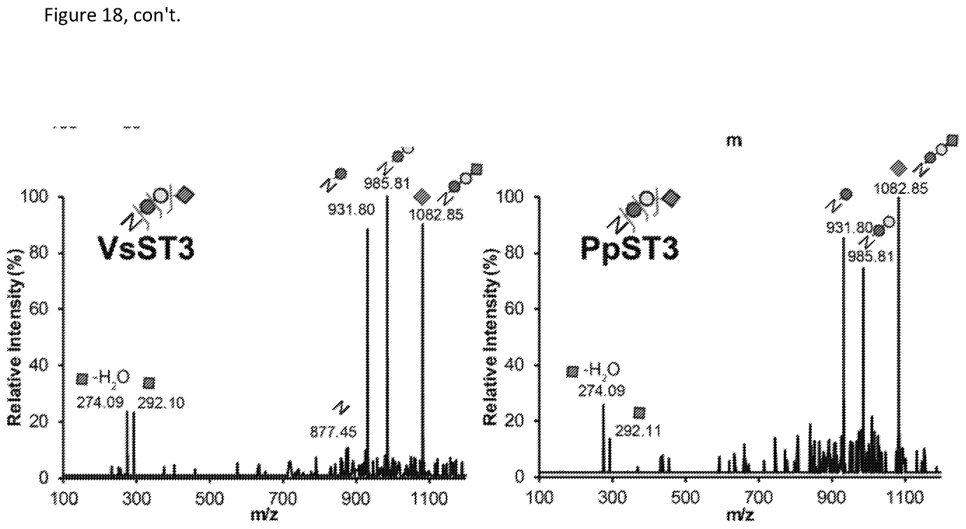

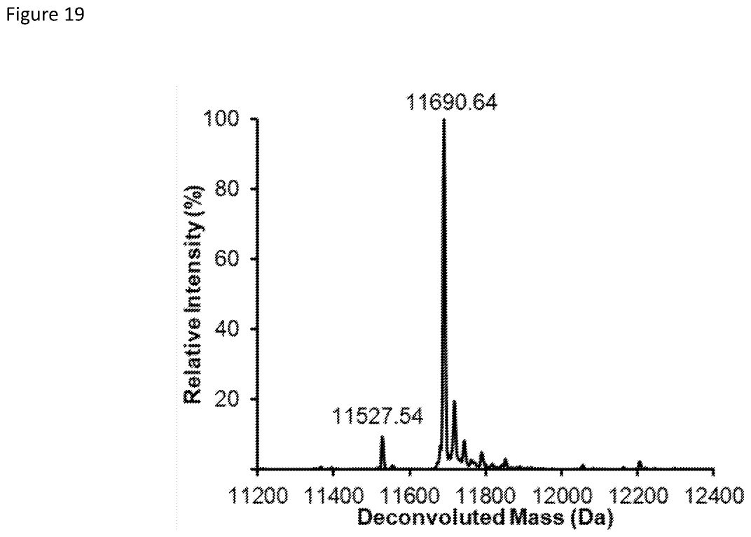

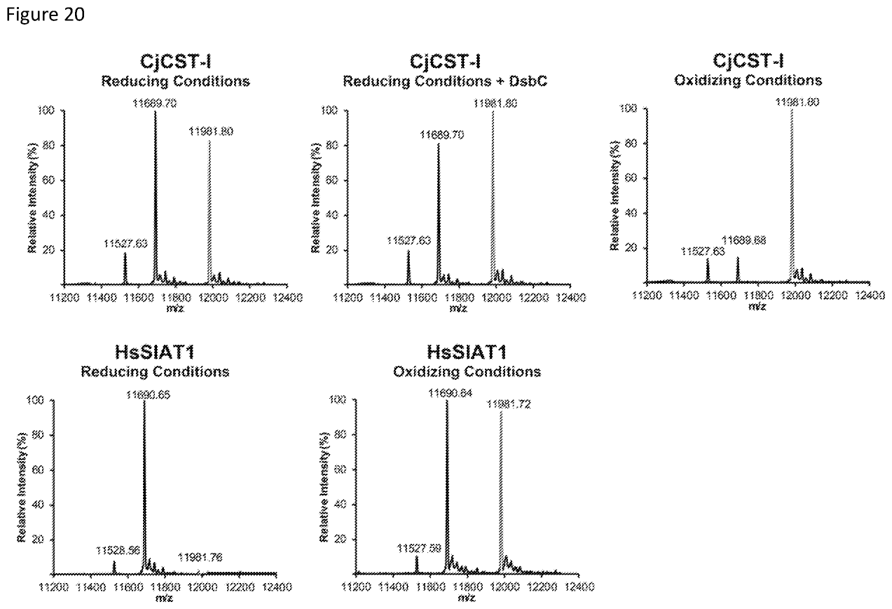

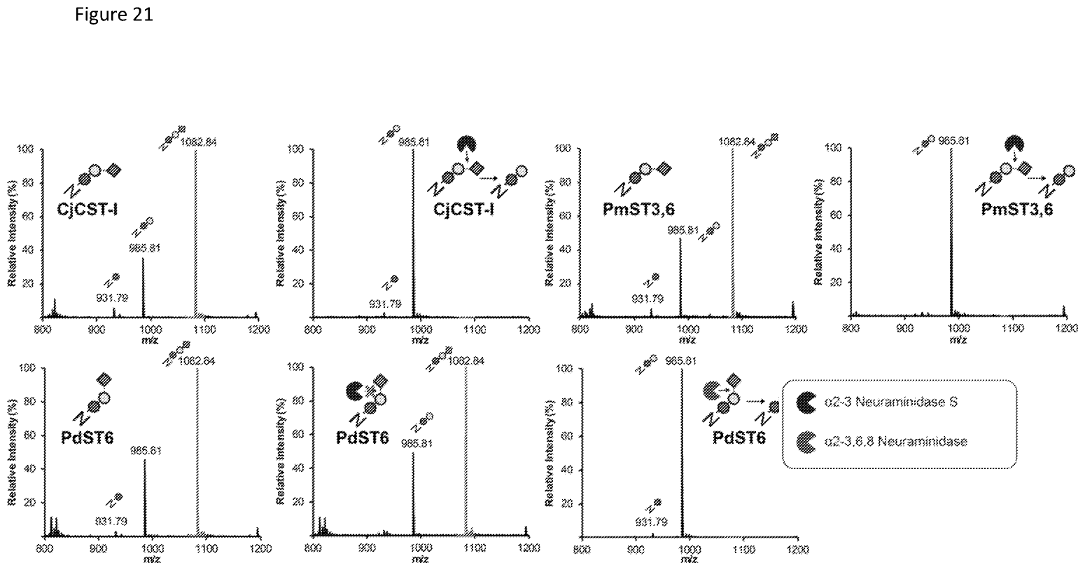

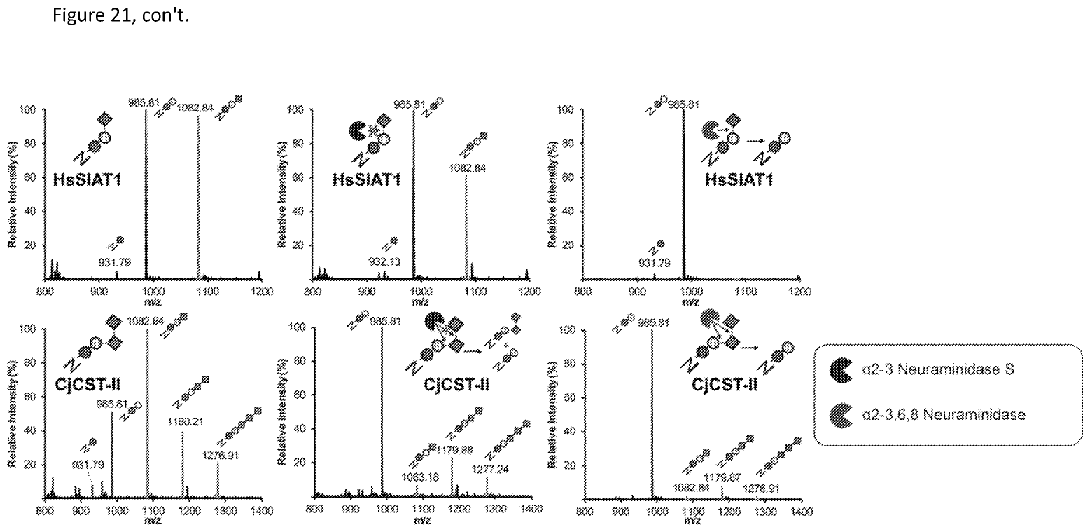

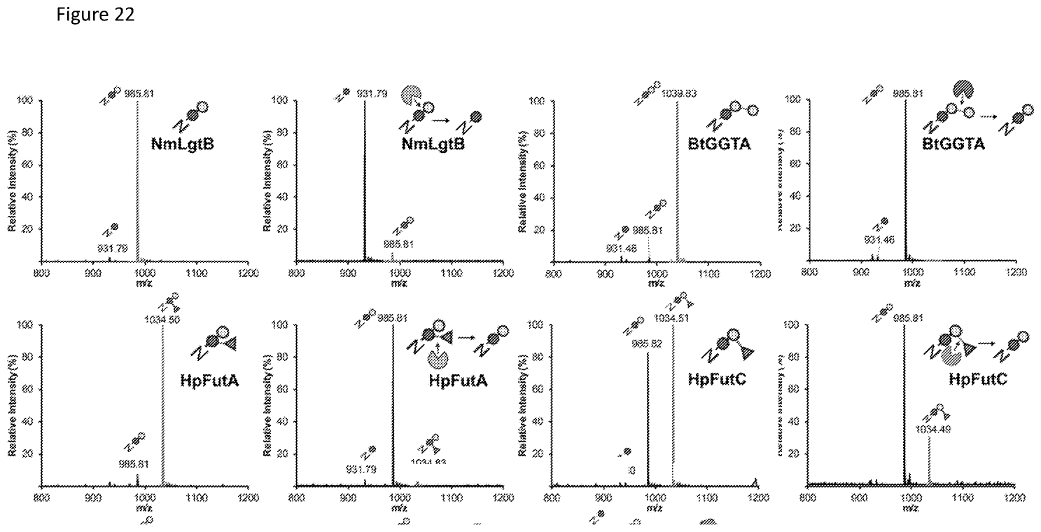

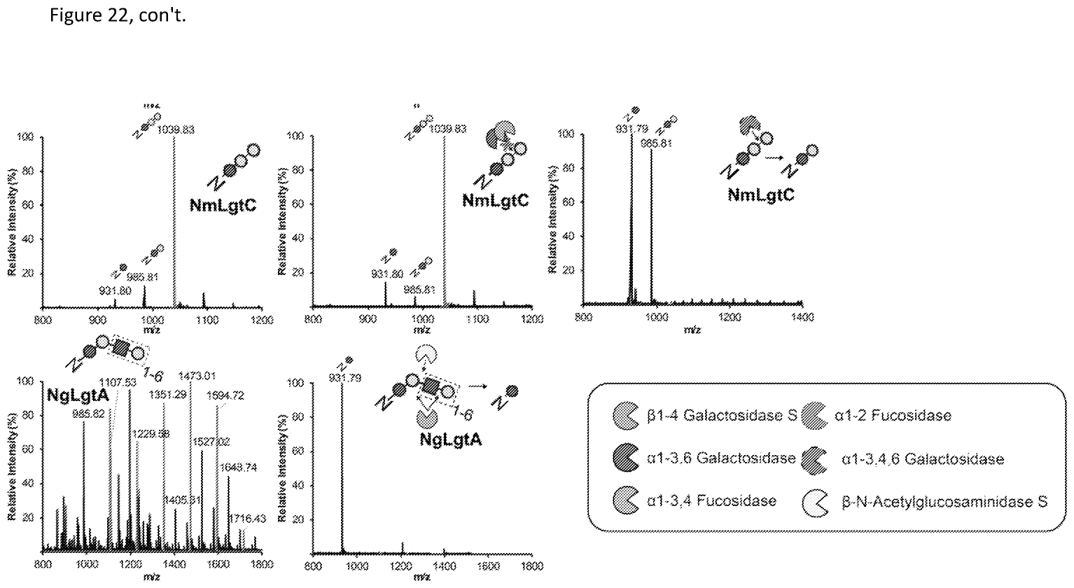

After designing pathways and selecting GTs, we used GlycoPRIME to synthesize and assemble three-enzyme biosynthetic pathways containing ApNGT, NmLgtB, and each of the 15 GTs described above. We first optimized and demonstrated full-length, soluble expression of each GT (FIG. 3a and FIG. 6 and FIGS. 12 and 13). We then used the GlycoPRIME workflow to synthesize Im7-6, ApNGT, NmLgtB and GTs for glycan extension in separate CFPS reactions and then mixed these CFPS products and appropriate sugar donors to form IVG reactions. Remarkably, when IVG products were purified by Ni-NTA and analyzed by LC-MS(/MS), we observed intact Im7-6 mass shifts (FIG. 3 and FIG. 17) and fragmentation spectra of trypsinized glycopeptides (FIG. 18) consistent with the modification of the N-linked lactose installed by ApNGT and NmLgtB according to the hypothesized activities of all 15 GTs selected for elaboration of this structure except HdGlcNAcT (FIG. 19). While we did detect some activity from all eight sialyltranferases by intact protein and/or glycopeptide analysis, we found that CjCST-I and PdST6 provided the highest conversion of all α2-3 and α2-6 sialyltranferases, respectively (FIG. 17). This optimization demonstrates the ability of GlycoPRIME to quickly compare several biosynthetic pathways to determine the enzyme combinations that yield desired products. We also found that we could significantly increase the conversion of reactions containing CjCST-I and HsSIAT1 by conducting CFPS of those GTs in oxidizing conditions (FIG. 20). This result demonstrates the advantages provided by the open reaction environment of CFPS reactions for improving enzyme synthesis, including the synthesis of a human enzyme with disulfide bonds (HsSIAT1). Notably, we found that NgLgtA not only installed GlcNAc, but also worked in turn with NmLgtB to form a LacNAc polymer with up to 6 repeat units (FIG. 3). In addition to intact protein and glycopeptide LC-MS(/MS), we performed digestions of Im7-6 modified by ApNGT, NmLgtB, and PdST6, HsSIAT1, CjCST-I, HpFutA, HpFutC, NgLgtA, and BtGGTA using commercially available exoglycosidases (FIGS. 21 and 22). Our findings support the previously established linkage specificities of these enzymes (FIGS. 2, 3, and FIG. 8). Under these conditions, we found that PmST3,6 exhibited primarily α2-3 activity, which is consistent with previous reports⁶¹.

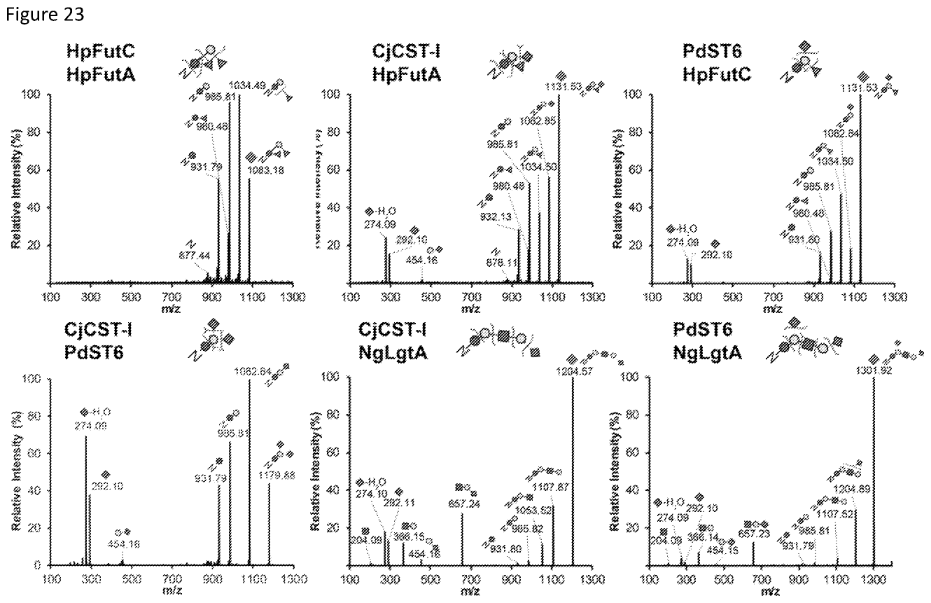

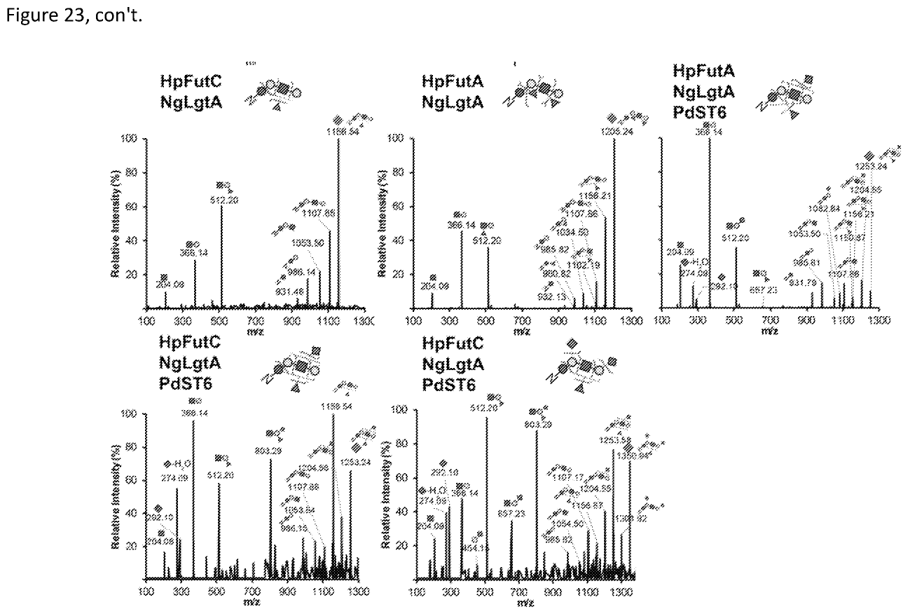

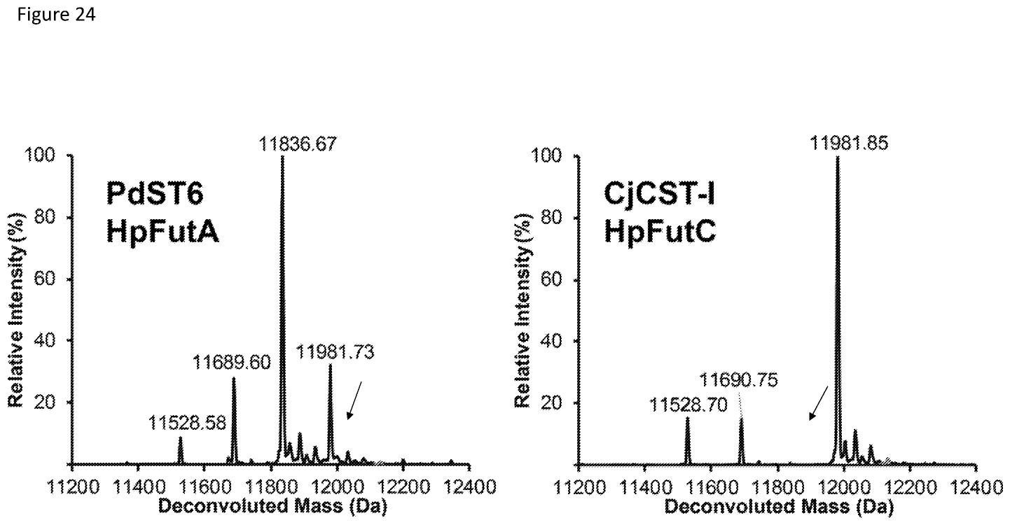

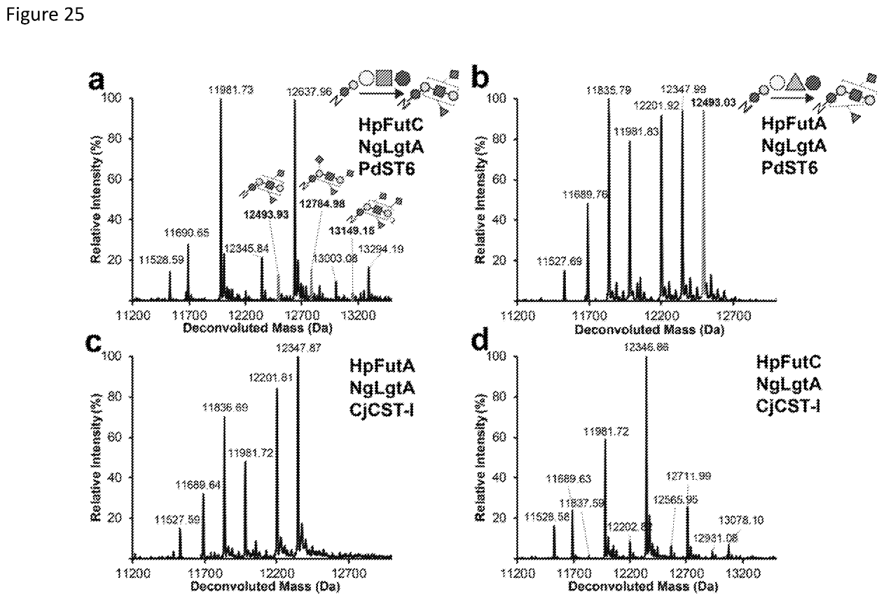

Having demonstrated the activity of diverse GTs using three-enzyme pathways, we pushed the GlycoPRIME system further to evaluate biosynthetic pathways containing four and five enzymes. Specifically, we aimed to synthesize sialylated and fucosylated lactose and LacNAc structures using combinations of HpFutA, HpFutC, CjCST-I, PdST6, and NgLgtA. Compared to the smaller glycans constructed above, these structures could provide greater specificity in a variety of applications including the targeting and inhibition of galectins, siglecs, and lectins on human and pathogenic cells^{44, 46, 57, 58} as well as the adjuvanting of vaccines by installing Lewis-X glycan structures that bind DC-SIGN receptors on dendritic cells⁶². While some combinations of these GTs have been used to create free oligosaccharides or glycolipids^{37-40, 63-65}, the products resulting from interactions between their specificities have not been systematically studied in the context of a protein substrate. We used GlycoPRIME to test all pairwise combinations of these five GTs, expressing each of them in separate CFPS reactions and then mixing two of those crude lysates in equal volumes with CFPS reactions containing 10 μM Im7-6, 0.4 μM ApNGT, and 2 μM NmLgtB. In our analysis of these IVG products, we observed intact protein (FIG. 3d) and glycopeptide fragmentation products (FIG. 23) indicating the synthesis of several interesting structures including difucosylated lactose, disialylated lactose, lactose variants with combinations of sialylation and fucosylation linkages, sialylated LacNAc structures with branching or only terminal Sia, and fucosylated LacNAc structures. Our analysis also revealed some possible specificity conflicts between the enzymes. For example, the combinations of CjCST-I with HpFutA and PdST6 with HpFutC yielded products which were both sialylated and fucosylated, but PdST6 with HpFutC and CjCST-I with HpFutC did not (FIG. 24). Furthermore, we observed that when HpFutC and NgLgtA are used together, only one fucose is added to the LacNAc backbone regardless of its length (FIG. 3d and FIG. 23). In contrast, when HpFutA and NgLgtA are combined, our observations suggest that both available Glc(NAc) residues may be modified; however, the shorter polymer length suggests that fucosylation with HpFutA may prohibit the continued growth of the LacNAc chain by NgLgtA (FIG. 3). While we focused here on testing reactions with all pathway enzymes acting simultaneously, sequential glycosylation reactions in vitro using a similar workflow could be used to further characterize these specificity conflicts and rigorously determine enzyme kinetics. To test the number of biosynthetic nodes GlycoPRIME can support, we constructed several five-enzyme glycosylation pathways using NgLgtA, one fucosyltransferase (HpFutA or HpFutC), and one sialyltransferase (CjCST-I or PdST6). While the complexity of these glycans did not allow us to unambiguously assign their structures, the intact protein mass shifts (FIG. 24) and fragmentation spectra (FIG. 23) from pathways containing NgLgtA, PdST6, and either HpFutA or HpFutC indicated the construction of LacNAc structures glycans which were both fucosylated and sialylated (FIG. 3d and FIGS. 23 and 25). Many glycans synthesized by these four- and five-enzyme combinations have not been previously described and further study will be required to understand the functional properties they provide.

### D. GlycoPRIME Pathways Function in Bacterial Production Systems

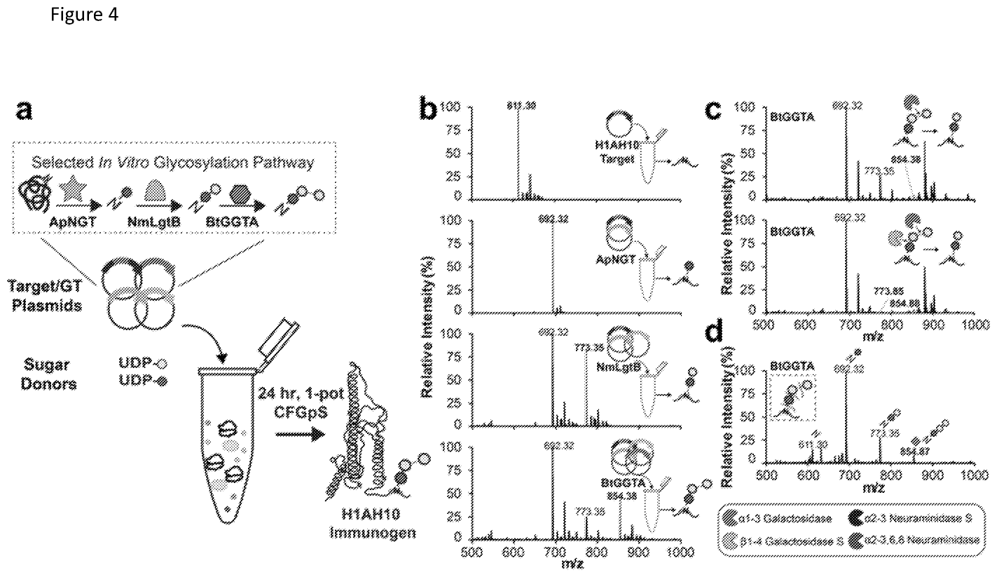

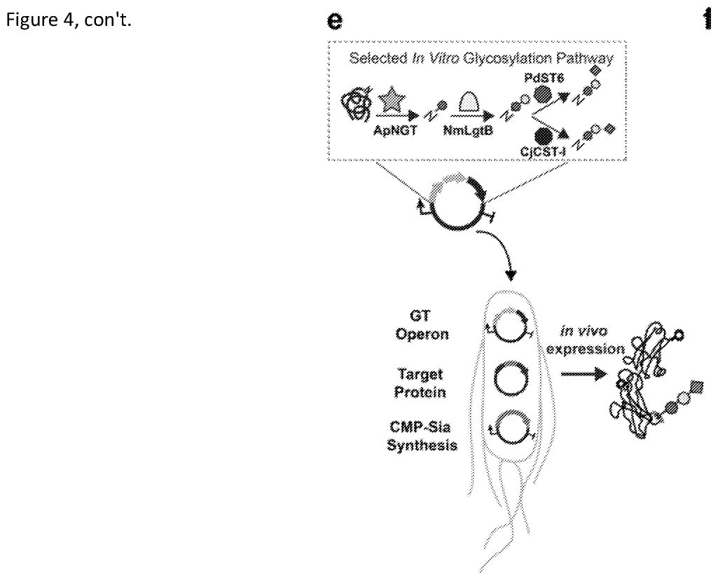

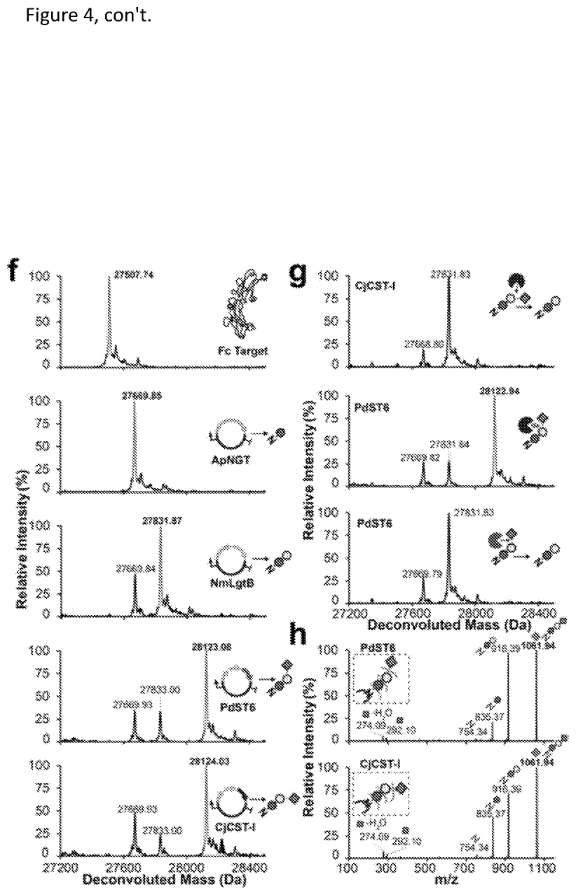

Having constructed and screened many new biosynthetic pathways using GlycoPRIME, we sought to demonstrate that the synthetic glycosylation pathways we discovered could be translated to new contexts within in vitro and in vivo bioproduction platforms to synthesize therapeutically relevant glycoproteins (FIG. 4).

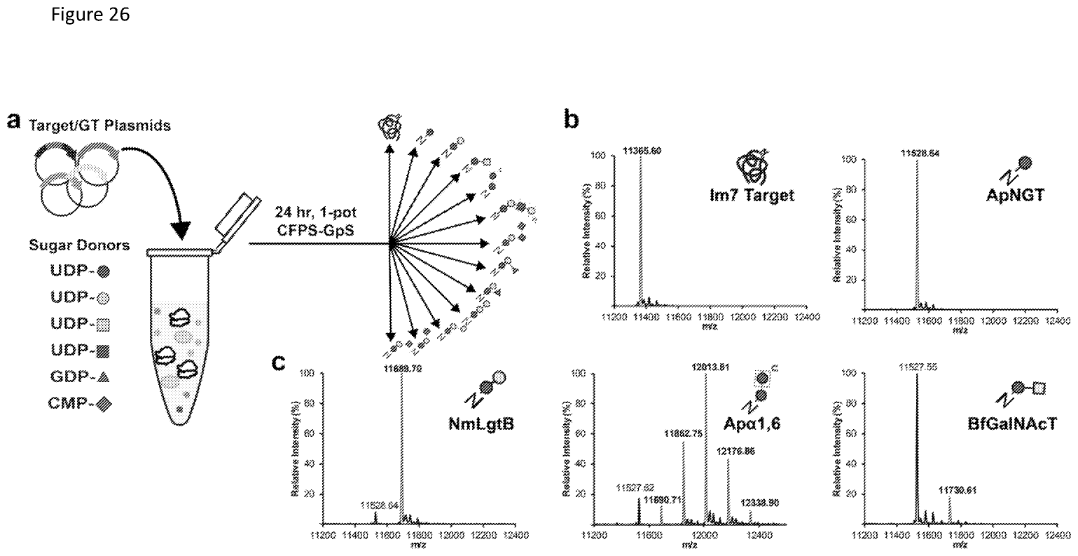

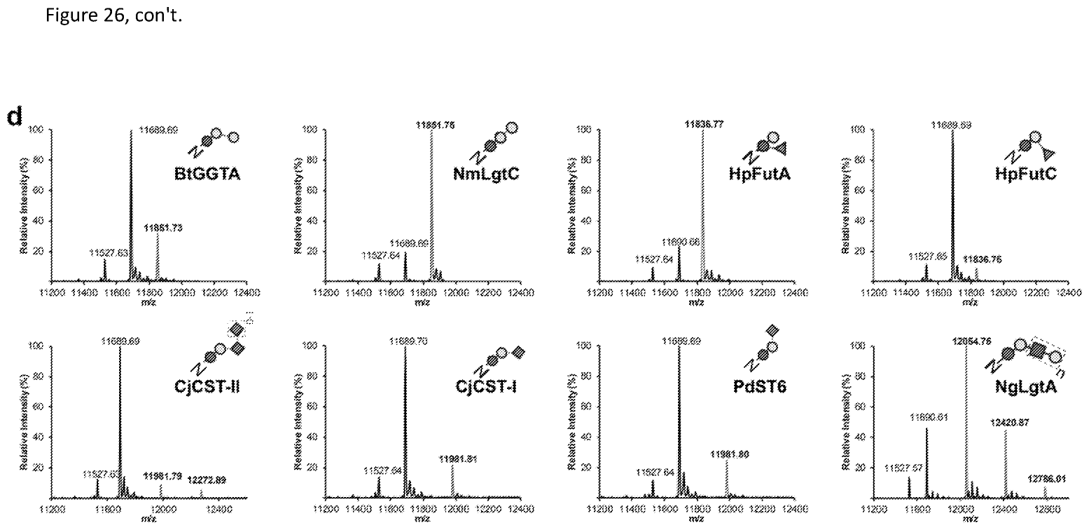

First, we aimed to translate the glycosylation pathways discovered using our two-pot GlycoPRIME system to a one-pot, coordinated cell-free protein synthesis driven glycoprotein synthesis (CFPS-GpS) platform. In CFPS-GpS, the target protein is co-expressed with GTs in the presence of sugar donors to simultaneously synthesize and glycosylate the glycoprotein of interest. This strategy provides an alternative and complementary approach to our previously reported one-pot cell-free glycoprotein synthesis (CFGpS) platforml8 by enabling expression of the glycosylation pathway enzymes in vitro rather than in vivo within the chassis strain before cell lysis. We validated our one-pot CFPS-GpS approach by mixing the Im7-6 target protein plasmid, sets of up to three GT plasmids based on 12 successful biosynthetic pathways developed in our two-pot GlycoPRIME screening, and appropriate sugar donors in one-pot CFPS-GpS reactions. In all reactions, we observed intact protein mass shifts consistent with the modification of Im7-6 with the same glycans observed in our two-pot system, albeit with lower efficiencies (FIG. 26). These results show that co-activation of target protein and GT synthesis with protein glycosylation is possible in one-pot, in vitro reactions, further simplifying and shortening the time required to produce glycoproteins compared to the two-pot GlycoPRIME format. Overall, CFPS-GpS uses only plasmids, commercially available small molecules, and an unenriched crude E. coli lysate to yield glycoprotein, enabling the versatile production of different glycoprotein targets and/or glycan structures according to the need or desired application by simply adding different plasmids to a single crude lysate source.

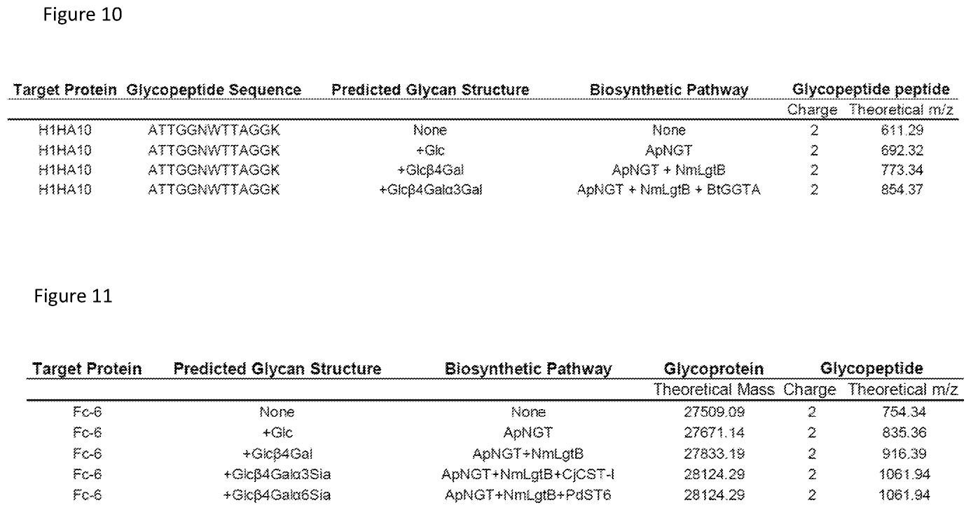

Having developed the CFPS-GpS approach, we aimed to synthesize and glycosylate an influenza vaccine candidate, H1HA10⁶⁶, with an αGal glycan motif using the biosynthetic pathway we discovered using GlycoPRIME (FIG. 4). We chose to demonstrate the αGal pathway on the H1HA10 model protein because H1HA10 is an effective immunogen that can be expressed in E. coli and the chemoenzymatic installation of αGal has been shown to act as an effective intramolecular adjuvant for other influenza vaccine candidates^{7, 67}. When we combined UDP-Glc, UDP-Gal, and plasmids encoding the H1HA10 protein ApNGT, NmLgtB, and BtGGTA in a one-pot CFPS-GpS reaction, we observed the installation of αGal on a tryptic peptide containing an engineered acceptor sequence at the N-terminus of H1HA10 (FIG. 4b). We further confirmed the linkages of this αGal glycan by exoglycosidase digestion and LC-MS/MS (FIGS. 4c-d and FIG. 10).

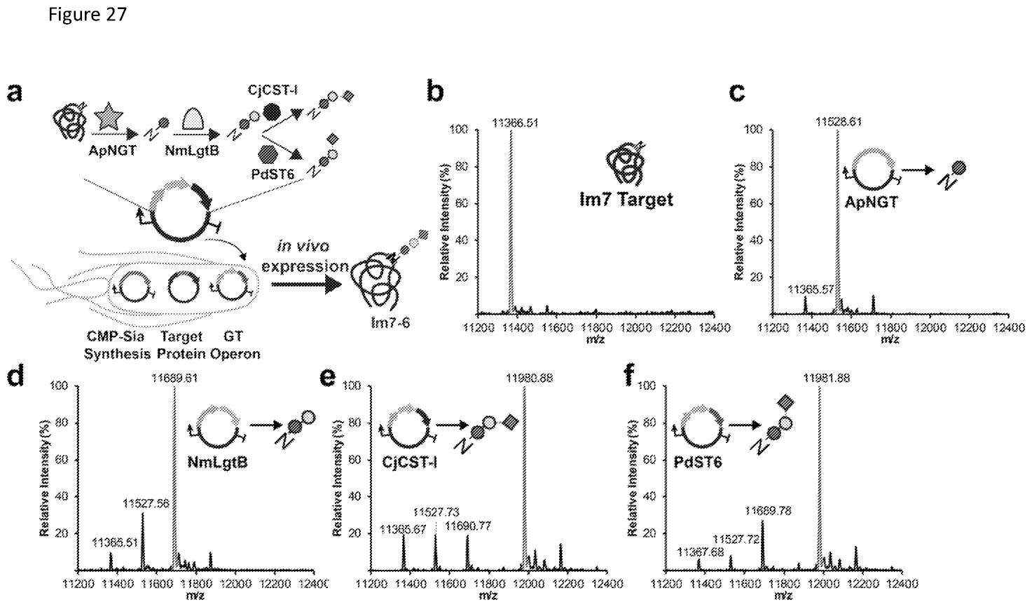

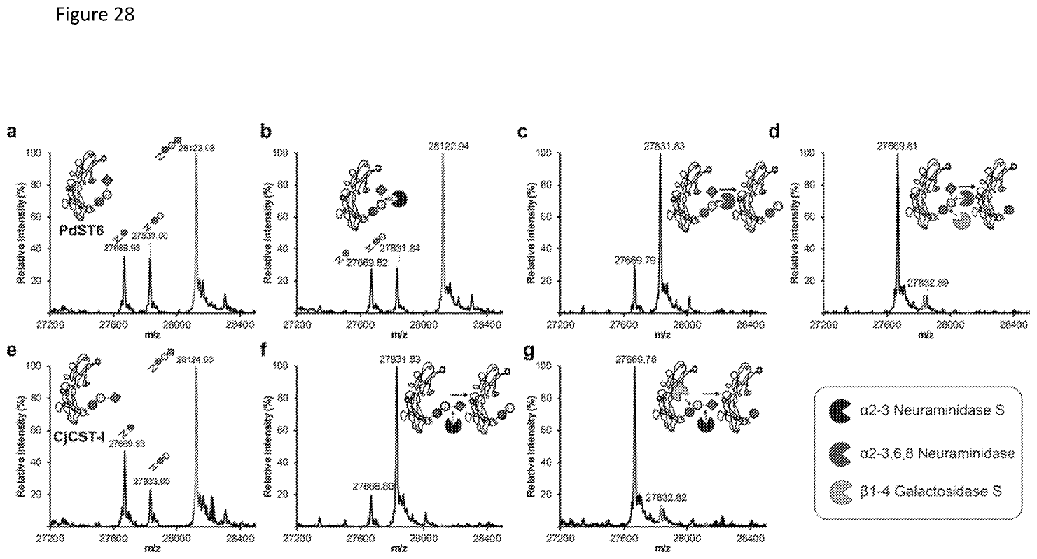

To demonstrate the transfer of pathways discovered using GlycoPRIME to living cells, we designed synthetic glycosylation systems to install N-linked 3′-sialyllactose and 6′-siallylactose onto the Fc region of human IgG1 in E. coli (FIG. 4). While glycoproteins with α2,8-linked polysialic acids have been produced in engineered E. coli²⁸, these glycans with distinct terminal sialic acid linkages and simplified, more homogeneous structures can provide unique and desirable properties for some applications of glycoprotein therapeutics^{5, 8, 34, 51} . To this end, we constructed a three-plasmid system composed of a constitutively expressed cytidine-5′-monophospho-N-acetylneuraminic acid (CMP-Sia) synthesis plasmid encoding the N. meningititus CMP-Sia synthase (ConNeuA); an Isopropyl β-D-1-thiogalactopyranoside (IPTG)-inducible target protein plasmid; and a GT operon plasmid encoding ApNGT, NmLgtB, and either CjCST-I or PdST6. The CMP-Sia synthesis plasmid is necessary because laboratory E. coli strains do not endogenously produce CMP-Sia. Based on previous reports^{28, 40}, we selected a K-12 E. coli strain carrying the nanT sialic acid transporter gene for intake of Sia supplemented to the media and knocked out the CMP-Sia aldolase gene (nanA) to prevent digestion of intracellular Sia, yielding CLM24ΔnanA. As with CFPS-GpS, we validated the in vivo synthesis of our target glycans using the Im7-6 model protein. When we transformed and induced our three-plasmid system in CLM24ΔnanA, we observed intact protein spectra consistent with the modification of Im7-6 with N-linked Glc by ApNGT, elaboration to lactose by NmLgtB, and elaboration to 3′-sialyllactose or 6′-siallylactose by CjCST-I or PdST6, respectively (FIG. 27). To synthesize Fc modified with these glycans, we replaced the Im7-6 target plasmid with a plasmid encoding Fc with an engineered acceptor sequence at the conserved human IgG1 glycosylation site at Asn297 (Fc-6)22. In this system, we observed intact protein MS, MS/MS peptide fragmentation, and exoglycosidase digestions consistent with the expected installation of Glc, lactose, and either 3′-sialyllactose or 6′-sialyllactose onto Fc-6 according to the GT operon supplied (FIG. 4f-h, FIG. 28, and FIG. 11). Further investigations will be required to assess the efficacy of the αGal epitope as an adjuvant for H1HA10 and the therapeutic effects of minimal sialic acid motifs on Fc. However, our findings clearly demonstrate that useful glycosylation pathways identified in the GlycoPRIME workflow can be quickly and easily translated to bacterial cell-free and cell-based expression platforms for production of therapeutically relevant glycoproteins.

### E. Discussion

This work establishes and demonstrates the utility of the GlycoPRIME platform, a cell-free workflow for the modular synthesis, assembly, and discovery of multienzyme glycosylation pathways. GlycoPRIME has several key features. First, by removing the need for LLO production in living cells, GlycoPRIME is the first system to enable the biosynthesis of glycosylation target, GTs, and glycoproteins entirely in vitro. This approach shifts the design-build test unit from a living cell line to a cell-free lysate. We demonstrated the utility of GlycoPRIME by rapidly exploring 37 putative protein glycosylation pathways, 23 of which yielded unique glycosylation motifs.

Second, the use of ApNGT (a soluble, bacterial enzyme) to efficiently install a priming N-linked glucose onto glycoproteins was key to facilitating pathway assembly. By elaborating this glucose residue, we generated a diverse library of therapeutically relevant glycosylation motifs from the bottom-up in vitro. Of the 23 unique glycosylation motifs for which biosynthetic pathways were discovered in this work, several have been synthesized as free^{37-40, 63, 64} or lipid-linked^{37, 38} oligosaccharides or by remodeling existing glycoproteins^{6, 30, 42}; however, to our knowledge, only glucose^{16, 22, 28,} dextran¹⁶, lactose²⁸, LacNAc⁶⁵, and polysialyllactose28 have been previously produced as glycoprotein conjugates in bacterial systems. The 18 synthetic glycosylation pathways leading to novel glycan motifs on proteins discovered in this work represent the largest addition made by any single bacterial glycoengineering study to date. Specifically, we developed the first bacterial biosynthesis pathways that yield proteins bearing N-linked 3′-siallylactose, 6′-siallylactose, the αGal epitope, pyruvylated lactose, 2′-fucosyllactose (Glcβ1-4Galα1-2Fuc), 3-fucosyllactose (Glcβ1-4[α1-3Fuc]Gal), as well as many other mono- or di-fucosylated and sialylated forms of lactose or LacNAc.

Third, biosynthetic pathways identified in GlycoPRIME can be implemented in new contexts and on new proteins for glycoprotein production in vitro and in the E. coli cytoplasm. Specifically, we demonstrated the synthesis of a candidate vaccine protein, H1HA10, modified with an αGal adjuvant motif in a one-pot CFPS-GpS reaction and the production of IgG1 Fc modified with 3′-siallylactose and 6′-siallylactose in E. coli (FIG. 4). While large-scale production and purification methods were not investigated, our work shows feasibility for translating pathways discovered by GlycoPRIME into relevant biomanufacturing expression systems. Furthermore, the use of ApNGT rather than OSTs makes these pathways attractive because they do not require transport across cellular membranes or membrane-associated components. These findings demonstrate the potential of GlycoPRIME to accelerate glycoengineering efforts and enable new applications in biotechnology, including on-demand production of glycoprotein therapeutics in combination with recent developments in distributed biomanufacturing systems^{21, 68, 69} and E. coli strains with reduced endotoxin levels^{21, 70, 71}.

While the glycosylation structures created in this work are less complex than natural human glycans, they still offer many promising applications. Potential applications include the development of imaging and other research reagents for fundamental studies of carbohydrate-binding proteins⁴⁴; glycan-based bacterial targeting⁶⁰, toxin neutralization⁵⁶, and adhesion prevention^{44, 45, 60;} improvement of glycoprotein therapeutic properties and trafficking^{5, 8, 28, 34, 42, 52}; new opportunities in functional biomaterials^{43, 57, 59}; modulation and inhibition of human galectins46 and siglecs^{46, 47;} and the development of new antigens^{49, 50, 53} and adjuvants for immunization^{6, 7, 33, 48, 55, 62} Although free oligosaccharides or small molecules can accomplish some of the functions above, the ability to build glycans site-specifically on glycoproteins as demonstrated in this work would enable a wide array of additional functionalities including targeting, antigen presentation, detection, imaging, and destruction^{6, 62}. Notably, further study will be required to assess the immunogenicity of the Asn-βGlc linkage created by ApNGT whose presence has only once been reported in mammalian systems⁷². If this linkage is immunogenic, the glycoprotein structures described here could still have significant impact in research, acute therapeutic applications, or immunization. Additionally, recent works have aimed to discover or engineer NGTs with relaxed sugar donor specificities (such as GlcNAc)^{32, 73} or combined these NGT variants with an acetyltransferase to produce N-linked GlcNAc³². We expect that these methods and future advancements will be compatible with most of the biosynthetic pathways described here because NmLgtB can modify Glc or GlcNAc acceptors³⁹.

Looking forward, GlycoPRIME provides a new way to discover, study, and optimize glycosylation pathways. For example, future applications could leverage the open and flexible reaction environment of GlycoPRIME to optimize enzyme stoichiometry for more homogeneous biosynthesis and to better understand GT specificities and kinetics. By enabling the synthesis and rapid assembly of enzymes that yield desired glycoproteins, GlycoPRIME is also poised to further expand the glycoengineering toolkit towards the production of glycoproteins on demand and by design. For example, recently reported methods to supplement lipid-associated glycans into cell-free synthesis reactions ¹⁸⁻²⁰ or produce GalNAcTs²² and OSTs¹⁹ in vitro present new opportunities to discover biosynthetic pathways yielding diverse glycans (N- and O-linked) with small modifications to the GlycoPRIME workflow. Finally, the diverse, yet simple set of glycans accessible by GlycoPRIME pathways could help elucidate the minimal motifs that provide desired glycoprotein properties. In sum, we expect that GlycoPRIME and biosynthetic pathways described in this work will accelerate the engineering of glycoproteins in bacterial systems, helping to merge the glycoscience and synthetic biology communities.

### F. Methods

Plasmid construction and molecular cloning. Details and sources of plasmids used in this study are shown in FIG. 5 with applicable database accession numbers. Full coding sequence regions with plasmid context are shown in FIG. 29. Codon-optimized DNA sequences encoding glycosylation targets and GTs in CFPS were synthesized as gene fragments or intact plasmids by Twist Bioscience, Integrated DNA Technologies, or Life Technologies. Gene fragments were inserted between Ndel and SalI restriction sites in the Kanamycin-resistant pJL1²² in vitro expression vector using polymerase chain reaction (PCR) amplification and Gibson assembly according to standard molecular biology techniques⁷⁴. Some GTs were produced with an N-terminal CAT-Strep-Linker (CSL) fusion sequence that has been shown to increase in vitro expression²² (see FIG. 29). Plasmids for expression of Im7-6 and Fc-6 glycosylation targets in the CLM24ΔnanA E. coli strain were generated by polymerase chain reaction (PCR) amplification of engineered forms of Im7 (Im7-6) and Fc (Fc-6) carrying optimized ApNGT glycosylation acceptor sequences and His-tags from pJL1.Im7-6 and pJL1.Fc-6²². These gene fragments were then placed into a pBR322 (ptrc99) backbone75 with Carbenicillin resistance and IPTG inducible expression between NcoI and HindIII restriction sites using Gibson assembly. Plasmids for expression of GT operons in E. coli were constructed by PCR amplification of ApNGT, NmLgtB, and CjCST-I or PdST6 from their pJL1 plasmid forms followed by Gibson assembly into a pMAF10 backbone²² with Trimethoprim resistance, a pBBR1 origin of replication, and arabinose inducible expression between NcoI and HindIII restriction sites. Strep-II tags, FLAG-tags, and ribosome binding sites designed using the RBS Calculator v2.076 for maximum translation initiation rate were inserted into these plasmids as shown in FIGS. 5 and 29. The pCon.NeuA plasmid for production of CMP-Sia in E. coli was generated by PCR amplification of NeuA from pTF77 followed by Gibson assembly into a pConYCG backbone with Kanamycin resistance and modified with a P32100 promoter for constitutive expression between the Nsil and SalI restriction sites.

Preparation of cell extracts for CFPS. CFPS of glycosylation enzymes and target proteins was performed using crude E. coli lysate from a recently described, high-yielding MG1655-derived E. coli strain C321.AA.75926 prepared using well-established methods^{22, 26}. Briefly, 1-liter cultures of E. coli cells were grown from a starting OD₆₀₀=0.08 in 2×YTPG media (yeast extract 10 g/l, tryptone 16 g/l, NaCl 5 g/l, K₂HPO₄ 7 g/l, KH₂PO₄ 3 g/l, and glucose 18 g/l, pH 7.2) in 2.5-liter Tunair flasks at 34° C. with shaking at 250 r.p.m. Cells were harvested on ice at OD₆₀₀=3.0 and pelleted by centrifugation at 5,000× g at 4° C. for 15 min. Cell pellets were washed three times with cold S30 buffer (10 mM Tris-acetate pH 8.2, 14 mM magnesium acetate, 60 mM potassium acetate, 2 mM dithiothreitol [DTT]) before being frozen on liquid nitrogen and then stored at −80° C. Cell pellets were thawed on ice and resuspended in 0.8 ml of S30 buffer per gram of wet cell weight and lysed in 1.4 ml aliquots on ice using a Q125 Sonicator (Qsonica) using three pulses (50% amplitude, 45 s on and 59 s off). After sonication, 4 μl of 1 M DTT was added to each aliquot. Each aliquot was centrifuged at 12,000× g and 4° C. for 10 min. The supernatant was incubated at 37° C. at 250 r.p.m. for 1 h and centrifuged at 10,000× g at 4° C. for 10 min. The clarified S12 lysate supernatant was then frozen on liquid nitrogen and stored at −80° C.

Cell-free protein synthesis. CFPS of glycosylation targets and GTs was performed using a well-established PANOx-SP crude lysate system26. Briefly, CFPS reactions contained 0.85 mM each of GTP, UTP, and CTP; 1.2 mM ATP; 170 μg/ml of E. coli tRNA mixture; 34 μg/ml folinic acid; 16 μg/ml purified T7 RNA polymerase; 2 mM of each of the 20 standard amino acids; 0.27 mM coenzyme-A (CoA); 0.33 mM nicotinamide adenine dinucleotide (NAD); 1.5 mM spermidine; 1 mM putrescine; 4 mM sodium oxalate; 130 mM potassium glutamate; 12 mM magnesium glutamate; 10 mM ammonium glutamate; 57 mM HEPES at pH=7.2; 33 mM phosphoenolpyruvate (PEP); 13.3 μg/ml DNA plasmid template encoding the desired protein in the pJL1 vector; and 27% v/v of E. coli crude lysate. E. coli total tRNA mixture (from strain MRE600) and phosphoenolpyruvate were purchased from Roche Applied Science. ATP, GTP, CTP, UTP, the 20 amino acids, and other materials were purchased from Sigma-Aldrich. Plasmid DNA for CFPS was purified from DH5-αE. coli strain (NEB) using ZymoPURE Midi Kit (Zymo Research). CFPS reactions under oxidizing conditions conducive to disulfide bond formation were performed similarly to standard CFPS reactions except for the use of a 30 minute preincubation of the lysate with 14.3 μM IAM and the addition of 4 mM oxidized L-glutathione GSSG, 1 mM reduced L-glutathione, and 3 μM of purified E. coli DsbC to the CFPS reaction78. All proteins were expressed in 15 μl batch CFPS reactions in 2.0 ml centrifuge tubes. For GlycoPRIME, CFPS reactions were incubated for 20 h at optimized temperatures for each protein (FIG. 6).

Cell-free protein synthesis driven glycoprotein synthesis. One-pot, CFPS-GpS was performed similarly to CFPS, except that CFPS-GpS reactions had a total volume of 50 μl and were supplemented with 2.5 mM of each appropriate activated sugar donor as well as multiple plasmid templates from the desired target protein and up to three GTs. CFPS-GpS reactions contained a total plasmid concentration of 10 nM, divided equally between each of the unique plasmids in the reaction. CFPS-GpS reactions were incubated for 24 h at 23° C. before purification by Ni-NTA magnetic beads for glycopeptide or intact protein analysis by LC-MS.

Quantification of CFPS yields. CFPS yields of glycosylation targets and GTs for GlycoPRIME were determined by supplementation of standard CFPS reactions with 10 μM leucine using established protocols^{22, 26}. Briefly, proteins produced in CFPS were precipitated and washed three times using 5% trichloroacetic acid (TCA) followed by quantification of incorporated radioactivity by a Microbeta2 liquid scintillation counter. Soluble yields were determined from fractions isolated after centrifugation at 12,000× g for 15 min at 4° C. Low levels of background radioactivity were measured in CFPS reactions containing no plasmid template and subtracted before calculation of protein yields.

Autoradiograms of CFPS reaction products. Autoradiograms of the soluble fractions of Im7-6 target and enzymes used in GlycoPRIME according to established methods²². Briefly, 2 μl CFPS reactions supplemented with 10 μM [14C]-leucine prior to the CFPS reaction and centrifuged at 12,000× g for 15 min at 4° C. after the CFPS reaction were separated using a 4-12% Bolt Bis-Tris Plus SDS-PAGE gel (Invitrogen) using MOPS buffer. The gels were stained using InstantBlue (Expedeon), imaged, and then dried overnight between cellophane films before a 72 h exposure to a Storage Phosphor Screen (GE Healthcare). The Phosphor Screen was imaged using a Typhoon FLA7000 imager (GE Healthcare) and the dried gels were imaged using a GelDoc XR+Imager (Bio-Rad) to assist with alignment to molecular weight standard ladder. SDS-PAGE and autoradiogram gel images were acquired using Image Lab Software version 6.0.0 and Typhoon FLA 7000 Control Software Version 1.2 Build 1.2.1.93, respectively.

In vitro glycosylation reactions. IVG reactions for GlycoPRIME were assembled in standard 0.2 ml tubes from the supernatant of completed CFPS reactions containing the Im7-6 target protein and indicated GTs centrifuged at 12,000× g for 10 min at 4° C. Target and enzyme yields were quantified and optimized by [¹⁴C]-leucine incorporation (FIG. 6). Standard IVG reactions contained 10 μM Im7-6 target, indicated amounts of up to five GTs forming a putative biosynthetic pathway, 10 mM MnCl2 (to provide the preferred metal cofactor for NmLgtB and other GTs), 23 mM HEPES buffer at pH=7.5, and 2.5 mM of each required nucleotide-activated sugar donor (according to previously characterized activities shown in FIG. 8). Each reaction contained a total volume of 32 μl with 25 μl of completed CFPS reactions (when necessary, the remaining CFPS reaction volume was filled by a completed CFPS reaction which had synthesized sfGFP). After assembly, IVG reactions containing up to two GTs were incubated for 24 h at 30° C. To increase conversion, IVG reactions containing more than two GTs were incubated for 24 h at 30° C., supplemented with an additional 2.5 mM of each activated sugar donor, and then incubated for an additional 24 h. When desired, both CFPS reactions and IVGs could be flash-frozen frozen after their respective incubation steps. After incubation, Im7-6 was purified from IVG reactions using magnetic His-tag Dynabeads (Thermo Fisher Scientific). The IVG reactions were diluted in 90 μl of Buffer 1 (50 mM NaH2PO4 and 300 mM NaCl, pH 8.0) and centrifuged at 12,000× g for 10 min at 4° C. This supernatant was incubated at room temperature for 10 min on a roller with 20 μl of beads which had been equilibrated with 120 μl of Buffer 1. The beads were then washed three times with 120 μl of Buffer 1 and then eluted using 70 μl of Buffer 1 with 500 mM imidazole. The samples were dialyzed against Buffer 2 (12.5 mM NaH2PO4 and 75 mM NaCl, pH 7.5) overnight using 3.5 kDa MWCO microdialysis cassettes (Pierce). Purification of one-pot CFPS-GpS reactions was completed similarly to IVG reactions.

Production of glycoproteins from living E. coli. The E. coli strain CLM24ΔnanA (genotype W3110 ΔwecA ΔnanA ΔwaaL::kan) was constructed to enable the intake and survival of sialic acid in the cytoplasm for the production of sialylated glycoproteins in vivo. CLM24ΔnanA was generated from W3110 using P1 transduction of the wecA::kan, nanA::kan, and waaL::kan alleles in that order, derived from the Keio collection⁷⁹. Between successive transductions, the kanamycin marker was removed using pE-FLP⁸⁰. As indicated, CLM24ΔnanA was sequentially transformed with the CMP-Sia production plasmid pCon.NeuA; a target protein plasmid pBR322.Im7-6 or pBR322.Fc-6; and a GT operon plasmid pMAF10 .NGT, pMAF10. ApNGT .NmLgtB, pMAF10. Cj CST-I.NmLgtB.ApNGT, or pMAF10.PdST6.NmLgtB.ApNGT by isolating individual clones with appropriate antibotics at each step. The completed strain was then used to inoculate a 5 ml overnight culture in LB media containing appropriate antibiotics which was then subcultured at OD₆₀₀=0.08 into 5 ml of fresh LB media supplemented with 5 mM N-Acetylneuraminic acid (sialic acid) purchased from Carbosynth and adjusted to pH=6.0 using NaOH and HC1. This culture was then grown at 37° C. with shaking at 250 r.p.m. GT operon expression was induced by supplementing the culture with 0.2% arabinose at OD₆₀₀ =0.4 and then target protein expression was induced at OD₆₀₀ =1.0 with 1 mM IPTG. After IPTG induction, the culture was grown overnight at 28° C. and 250 r.p.m. The cells were pelleted by centrifugation at 4° C. for 10 min at 4,000 x g, frozen on liquid nitrogen, and stored at −80° C. Cell pellets were thawed and resuspended in 630 μl of Buffer 1 with 5 mM imidazole and supplemented with 70 μl of 10 mg/ml lysozyme (Sigma), 1 μl (250 U) Benzonase (Millipore), and 7 μl of 100× Halt protease inhibitor (Thermo Fisher Scientific). After 15 min of thawing and resuspension, the cells were incubated for 15-60 min on ice, sonicated for 45 s at 50% amplitude, and then centrifuged at 12,000× g for 15 min. The supernatant was then incubated on a roller for 10 min at RT with 50 μl of His-tag Dynabeads which had been pre-equilibrated with 5 mM imidazole in Buffer 1. The beads were then washed three times with 1 ml of Buffer 1 containing 5 mM imidazole and then eluted with 70 μl of Buffer 1 with 500 mM imidazole by a 10 min incubation on a roller at RT. Samples were then dialyzed with 3.5 kDa MWCO microdialysis cassettes overnight against Buffer 2 before glycopeptide or glycoprotein processing and analysis for LC-MS.

LC-MS analysis of glycoprotein modification. Modification of intact glycoprotein targets was determined by LC-MS by injection of 5 μl (or about 5 pmol) of His-tag purified, dialyzed glycoprotein into a Bruker Elute UPLC equipped with an ACQUITY UPLC Peptide BEH C4 Column, 300Å, 1.7 μm, 2.1 mm×50 mm (186004495 Waters Corp.) with a 10 mm guard column of identical packing (186004495 Waters Corp.) coupled to an Impact-II UHR TOF Mass Spectrometer (Bruker Daltonics, Inc.). Before injection, Fc samples were reduced with 50 mM DTT. Liquid chromatography was performed using 100% H2O and 0.1% formic acid as Solvent A and 100% acetonitrile and 0.1% formic acid as Solvent B at a flow rate of 0.5 mL/min and a 50° C. column temperature. An initial condition of 20% B was held for 1 min before elution of the proteins of interest during a 4 min gradient from 20% to 50% B. The column was washed and equilibrated by 0.5 min at 71.4% B, 0.1 min gradient to 100% B, 2 min wash at 100% B, 0.1 min gradient to 20% B, and then a 2.2 min hold at 20% B, giving a total 10 min run time. An MS scan range of 100-3000 m/z with a spectral rate of 2 Hz was used. External calibration was performed prior to data collection.

LC-MS analysis of glycopeptide modification. Glycopeptides for LC-MS(/MS) analysis were prepared by digesting His-tag purified, dialyzed glycosylation targets with 0.0044 μg/μl MS Grade Trypsin (Thermo Fisher Scientific) at 37° C. overnight. Before injection, H1HA10 samples were reduced by incubation with 10 mM DTT for 2 h. LC-MS(/MS) was performed by injection of 2 μl (or about 2 pmol) of digested glycopeptides into a Bruker Elute UPLC equipped with an ACQUITY UPLC Peptide BEH C18 Column, 300Å, 1.7 μm, 2.1 mm×100 mm (186003686 Waters Corp.) with a 10 mm guard column of identical packing (186004629 Waters Corp.) coupled to an Impact-II UHR TOF Mass Spectrometer. Liquid chromatography was performed using 100% H2O and 0.1% formic acid as Solvent A and 100% acetonitrile and 0.1% formic acid as Solvent B at a flow rate of 0.5 mL/min and a 40° C. column temperature. An initial condition of 0% B was held for 1 min before elution of the peptides of interest during a 4 min gradient to 50% B. The column was washed and equilibrated by a 0.1 min gradient to 100% B, a 2 min wash at 100% B, a 0.1 min gradient to 0% B, and then a 1.8 min hold at 0% B, giving a total 9 min run time. LC-MS/MS of glycopeptides was performed to confirm that GT modifications were in accordance with previously characterized specificities. Pseudo multiple reaction monitoring (MRM) MS/MS fragmentation was targeted to theoretical glycopeptide masses corresponding to detected intact protein MS peaks. All glycopeptides were fragmented using a collisional energy of 30 eV with a window of ±2 m/z from targeted m/z values. Theoretical protein, peptide, and sugar ion masses derived from expected glycosylation structures are shown in FIGS. 7 and 9-11. For LC-MS and LC-MS/MS of glycopeptides, a scan range of 100-3000 m/z with a spectral rate of 8 Hz was used. External calibration was performed prior to data collection.

Exoglycosidase digestions. When possible, sugar linkages installed by various GTs and biosynthetic pathways were confirmed by exoglycosidase digestion using commercially available enzymes from New England Biolabs with well-characterized activities. As indicated in figures and figure legends, glycoproteins or glycopeptides were incubated with exoglycosidases for at least 4 h at 37° C. using buffers and digestion conditions suggested by the manufacturer. The exoglycosidases and associated product numbers used in this study are: β1-4 Galactosidase S (P0745S); α1-3,6 Galactosidase (P0731S); α1-3,4 Fucosidase (P0769S); and α1-2 Fucosidase (P0724S); α1-3,4,6 Galactosidase (P0747S); β-N-Acetylglucosaminidase S (P0744S); α2-3 Neuraminidase S (P0743S); and α2-3,6,8 Neuraminidase (P0720S).

LC-MS(/MS) data analysis. LC-MS(/MS) data was collected using Bruker Compass Hystar v4.1 and analyzed using Bruker Compass Data Analysis v4.1 (Bruker Daltonics, Inc.). Glycopeptide MS and intact glycoprotein MS spectra were averaged across the full elution times of the glycosylated and aglycosylated glycoforms (as determined by extracted ion chromatograms of theoretical glycopeptide and glycoprotein charge states). MS spectra for intact glycoproteins was then analyzed by Data Analysis maximum entropy deconvolution from the full m/z scan range of 100-2,000 into a mass range of 10,000-14,000 Da for Im7-6 samples or 27,000-29,000 Da for Fc-6 samples. Representative LC-MS/MS spectra from MRM fragmentation were selected and annotated manually. Observed glycopeptide m/z and intact protein deconvoluted masses are annotated in figures and theoretical values are shown in FIGS. 7 and 9-11. LC-MS(/MS) data was exported from Bruker Compass Data Analysis and plotted in Microsoft Excel 365.

Statistical Information. FIG. legends indicate exact sample numbers for means, standard deviations (error bars), and representative data for each experiment. No tests for statistical significance or animal subjects were used in this study.

Data availability. All data generated or analyzed during this study are included or are available from the inventors upon reasonable request. The source data underlying the averages reported in FIG. 6 are provided as a Source Data file available at Kightlinger et al., Nature Communications, 10, Article No. 5404 (Nov. 27, 2019), herein incorporated by reference in its entirety.

### G. References Cited in Example 1

1. Helenius, A. & Aebi, M. Intracellular functions of N-linked glycans. Science (New York, N.Y.) 291, 2364-2369 (2001).

2. Khoury, G. A., Baliban, R. C. & Floudas, C. A. Proteome-wide post-translational modification statistics: frequency analysis and curation of the swiss-prot database. Scientific reports 1, 90 (2011).

3. Sethuraman, N. & Stadheim, T. A. Challenges in therapeutic glycoprotein production. Current Opinions in Biotechnology 17, 341-346 (2006).

4. Elliott, S. et al. Enhancement of therapeutic protein in vivo activities through glycoengineering. Nature Biotechnology 21, 414-421 (2003).

5. Varki, A. Sialic acids in human health and disease. Trends in molecular medicine 14, 351-360 (2008).

6. Abdel-Motal, U. M. et al. Increased immunogenicity of HIV-1 p24 and gp120 following immunization with gp120/p24 fusion protein vaccine expressing alpha-gal epitopes. Vaccine 28, 1758-1765 (2010).

7. Abdel-Motal, U. M., Guay, H. M., Wigglesworth, K., Welsh, R. M. & Galili, U. Immunogenicity of influenza virus vaccine is increased by anti-gal-mediated targeting to antigen-presenting cells. Journal of virology 81, 9131-9141 (2007).

8. Lin, C. -W. et al. A common glycan structure on immunoglobulin G for enhancement of effector functions. Proceedings of the National Academy of Sciences USA 112, 10611-10616 (2015).

9. Keys, T. G. & Aebi, M. Engineering protein glycosylation in prokaryotes. Current Opinion in Systems Biology 5, 23-31 (2017).

10. Li, H. et al. Optimization of humanized IgGs in glycoengineered Pichia pastoris. Nature Biotechnology 24, 210-215 (2006).

11. Yang, Z. et al. Engineered CHO cells for production of diverse, homogeneous glycoproteins. Nature Biotechnology 33, 842-844 (2015).

12. Wang, L. -X. & Amin, M. N. Chemical and Chemoenzymatic Synthesis of Glycoproteins for Deciphering Functions. Chemistry & Biology 21, 51-66 (2014).

13. Valderrama-Rincon, J. D. et al. An engineered eukaryotic protein glycosylation pathway in Escherichia coli. Nature Chemical Biology 8, 434-436 (2012).

14. Wacker, M. et al. N-linked glycosylation in Campylobacter jejuni and its functional transfer into E. coli. Science (New York, N.Y.) 298, 1790-1793 (2002).

15. Feldman, M. F. et al. Engineering N-linked protein glycosylation with diverse O antigen lipopolysaccharide structures in Escherichia coli. Proceedings of the National Academy of Sciences of the United States of America 102, 3016-3021 (2005).

16. Cuccui, J. et al. The N-linking glycosylation system from Actinobacillus pleuropneumoniae is required for adhesion and has potential use in glycoengineering. Open biology 7 (2017).

17. Naegeli, A. et al. Molecular analysis of an alternative N-glycosylation machinery by functional transfer from Actinobacillus pleuropneumoniae to Escherichia coli. Journal of Biological Chemistry 289, 2170-2179 (2014).

18. Jaroentomeechai, T. et al. Single-pot glycoprotein biosynthesis using a cell-free transcription-translation system enriched with glycosylation machinery. Nature Communications 9, 2686 (2018).

19. Schoborg, J. A. et al. A cell-free platform for rapid synthesis and testing of active oligosaccharyltransferases. Biotechnology and bioengineering (2017).

20. Guarino, C. & DeLisa, M. P. A prokaryote-based cell-free translation system that efficiently synthesizes glycoproteins. Glycobiology 22, 596-601 (2012).

21. Stark, J. C. et al. On-demand, cell-free biomanufacturing of conjugate vaccines at the point-of-care. Preprint at https://www.biorxiv. org/content/biorxiv/early/2019/2006/2024/681841.full.pdf (2019).

22. Kightlinger, W. et al. Design of glycosylation sites by rapid synthesis and analysis of glycosyltransferases. Nature Chemical Biology 14, 627-635 (2018).

23. Karim, A. S. & Jewett, M. C. A cell-free framework for rapid biosynthetic pathway prototyping and enzyme discovery. Metabolic Engineering 36, 116-126 (2016).

24. Dudley, Q. M., Anderson, K. C. & Jewett, M. C. Cell-Free Mixing of Escherichia coli Crude Extracts to Prototype and Rationally Engineer High-Titer Mevalonate Synthesis. ACS synthetic biology 5, 1578-1588 (2016).

25. Dudley, Q. M., Karim, A. S. & Jewett, M. C. Cell-free metabolic engineering: Biomanufacturing beyond the cell. Biotechnology journal 10, 69-82 (2015).

26. Martin, R. W. et al. Cell-free protein synthesis from genomically recoded bacteria enables multisite incorporation of noncanonical amino acids. Nature Communications 9, 1203 (2018).

27. Napiórkowska, M. et al. Molecular basis of lipid-linked oligosaccharide recognition and processing by bacterial oligosaccharyltransferase. Nature Structural and Molecular Biology 24, 1100 (2017).

28. Keys, T. G. et al. A biosynthetic route for polysialylating proteins in Escherichia coli. Metabolic Engineering 44, 293-301 (2017).

29. Schwarz, F., Fan, Y. -Y., Schubert, M. & Aebi, M. Cytoplasmic N-Glycosyltransferase of Actinobacillus pleuropneumoniae Is an Inverting Enzyme and Recognizes the NX(S/T) Consensus Sequence. Journal of Biological Chemistry 286, 35267-35274 (2011).

30. Lomino, J. V. et al. A two-step enzymatic glycosylation of polypeptides with complex N-glycans. Bioorganic & Medicinal Chemistry 21, 2262-2270 (2013).

31. Song, Q. et al. Production of homogeneous glycoprotein with multi-site modifications by an engineered N-glycosyltransferase mutant. Journal of Biological Chemistry (2017).

32. Xu, Y. et al. A novel enzymatic method for synthesis of glycopeptides carrying natural eukaryotic N-glycans. Chemical Communications 53, 9075-9077 (2017).

33. Phanse, Y. et al. A systems approach to designing next generation vaccines: combining alpha-galactose modified antigens with nanoparticle platforms. Scientific reports 4, 3775 (2014).

34. Bork, K., Horstkorte, R. & Weidemann, W. Increasing the sialylation of therapeutic glycoproteins: The potential of the sialic acid biosynthetic pathway. Journal of Pharmaceutical Sciences 98, 3499-3508 (2009).

35. Passmore, I. J., Andrejeva, A., Wren, B. W. & Cuccui, J. Cytoplasmic glycoengineering of Apx toxin fragments in the development of Actinobacillus pleuropneumoniae glycoconjugate vaccines. BMC veterinary research 15, 6 (2019).

36. Ban, L. et al. Discovery of glycosyltransferases using carbohydrate arrays and mass spectrometry. Nature Chemical Biology 8, 769-773 (2012).

37. Dumon, C., Samain, E. & Priem, B. Assessment of the Two Helicobacter pylori α-1,3-Fucosyltransferase Ortholog Genes for the Large-Scale Synthesis of LewisX Human Milk Oligosaccharides by Metabolically Engineered Escherichia coli. Biotechnology Progress 20, 412-419 (2004).

38. Huang, D. et al. Metabolic engineering of Escherichia coli for the production of 2′-fucosyllactose and 3-fucosyllactose through modular pathway enhancement. Metabolic Engineering 41, 23-38 (2017).

39. Li, Y. et al. Donor substrate promiscuity of bacterial beta1-3-N-acetylglucosaminyltransferases and acceptor substrate flexibility of beta1-4-galactosyltransferases. Bioorganic and Medicinal Chemistry 24, 1696-1705 (2016).

40. Priem, B., Gilbert, M., Wakarchuk, W. W., Heyraud, A. & Samain, E. A new fermentation process allows large-scale production of human milk oligosaccharides by metabolically engineered bacteria. Glycobiology 12, 235-240 (2002).

41. Aanensen, D. M., Mavroidi, A., Bentley, S. D., Reeves, P. R. & Spratt, B. G. Predicted Functions and Linkage Specificities of the Products of the Streptococcus pneumoniae Capsular Biosynthetic Loci. Journal of bacteriology 189, 7856-7876 (2007).

42. Lindhout, T. et al. Site-specific enzymatic polysialylation of therapeutic proteins using bacterial enzymes. Proceedings of the National Academy of Sciences 108, 7397-7402 (2011).

43. Sgambato, A. et al. Different Sialoside Epitopes on Collagen Film Surfaces Direct Mesenchymal Stem Cell Fate. ACS Applied Materials & Interfaces 8, 14952-14957 (2016).

44. Imberty, A. & Varrot, A. Microbial recognition of human cell surface glycoconjugates. Curr Opin Struct Biol 18, 567-576 (2008).

45. Barthelson, R., Mobasseri, A., Zopf, D. & Simon, P. Adherence of Streptococcus pneumoniae to respiratory epithelial cells is inhibited by sialylated oligosaccharides. Infection and immunity 66, 1439-1444 (1998).

46. Rabinovich, G. A. & Toscano, M. A. Turning “sweet” on immunity: galectin-glycan interactions in immune tolerance and inflammation. Nature Reviews Immunology 9, 338 (2009).

47. O'Reilly, M. K. & Paulson, J. C. Siglecs as targets for therapy in immune-cell-mediated disease. Trends in Pharmacological Sciences 30, 240-248 (2009).

48. Chen, W. C. et al. Antigen Delivery to Macrophages Using Liposomal Nanoparticles Targeting Sialoadhesin/CD169. PloS one 7, e39039 (2012).

49. Ragupathi, G. et al. Induction of antibodies against GD3 ganglioside in melanoma patients by vaccination with GD3-lactone-KLH conjugate plus immunological adjuvant QS-21. International Journal of Cancer 85, 659-666 (2000).

50. Pan, Y., Chefalo, P., Nagy, N., Harding, C. & Guo, Z. Synthesis and immunological properties of N-modified GM3 antigens as therapeutic cancer vaccines. Journal of Medicinal Chemistry 48, 875-883 (2005).

51. Meuris, L. et al. GlycoDelete engineering of mammalian cells simplifies N-glycosylation of recombinant proteins. Nature Biotechnology 32, 485-489 (2014).

52. Chen, W. C. et al. In vivo targeting of B-cell lymphoma with glycan ligands of CD22. Blood 115, 4778-4786 (2010).

53. Zou, W. et al. Bioengineering of surface GD3 ganglioside for immunotargeting human melanoma cells. Journal of Biological Chemistry (2004).

54. Higuchi, Y. et al. A rationally engineered yeast pyruvyltransferase Pvg1p introduces sialylation-like properties in neo-human-type complex oligosaccharide. Scientific reports 6, 26349 (2016).

55. Deguchi, T. et al. Increased Immunogenicity of Tumor-Associated Antigen, Mucin 1, Engineered to Express α-Gal Epitopes: A Novel Approach to Immunotherapy in Pancreatic Cancer. Cancer Research 70, 5259-5269 (2010).

56. Kitov, P.I. et al. Shiga-like toxins are neutralized by tailored multivalent carbohydrate ligands. Nature 403, 669 (2000).

57. Beer, M.V. et al. The Next Step in Biomimetic Material Design: Poly-LacNAc-Mediated Reversible Exposure of Extra Cellular Matrix Components. Advanced Healthcare Materials 2, 306-311 (2013).

58. Laaf, D., Bojarová, P., Pelantová, H., Kěn, V. & Elling, L. Tailored Multivalent Neo-Glycoproteins: Synthesis, Evaluation, and Application of a Library of Galectin-3-Binding Glycan Ligands. Bioconjugate chemistry 28, 2832-2840 (2017).

59. Kalovidouris, S. A., Gama, C. I., Lee, L. W. & Hsieh-Wilson, L. C. A Role for Fucose α(1-2) Galactose Carbohydrates in Neuronal Growth. Journal of the American Chemical Society 127, 1340-1341 (2005).

60. Yu, Y. et al. Human Milk Contains Novel Glycans That Are Potential Decoy Receptors for Neonatal Rotaviruses. Molecular & Cellular Proteomics 13, 2944-2960 (2014).

61. Yu, H. et al. A Multifunctional Pasteurella multocida Sialyltransferase: A Powerful Tool for the Synthesis of Sialoside Libraries. Journal of the American Chemical Society 127, 17618-17619 (2005).

62. Wang, J. et al. Lewis X oligosaccharides targeting to DC-SIGN enhanced antigen-specific immune response. Immunology 121, 174-182 (2007).

63. Yavuz, E., Maffioli, C., Ilg, K., Aebi, M. & Priem, B. Glycomimicry: display of fucosylation on the lipo-oligosaccharide of recombinant Escherichia coli K12. Glycoconjugate Journal 28, 39-47 (2011).

64. Ilg, K., Yavuz, E., Maffioli, C., Priem, B. & Aebi, M. Glycomimicry: display of the GM3 sugar epitope on Escherichia coli and Salmonella enterica sv Typhimurium. Glycobiology 20, 1289-1297 (2010).

65. Hug, I. et al. Exploiting Bacterial Glycosylation Machineries for the Synthesis of a Lewis Antigen-containing Glycoprotein. Journal of Biological Chemistry 286, 37887-37894 (2011).

66. Mallajosyula, V. V. A. et al. Influenza hemagglutinin stem-fragment immunogen elicits broadly neutralizing antibodies and confers heterologous protection. Proceedings of the National Academy of Sciences USA 111, E2514-E2523 (2014).

67. Chen, W. A. et al. Addition of alphaGal HyperAcute technology to recombinant avian influenza vaccines induces strong low-dose antibody responses. PloS one 12, e0182683 (2017).

68. Pardee, K. et al. Portable, On-Demand Biomolecular Manufacturing. Cell 167, 248-259.e212 (2016).

69. Crowell, L. E. et al. On-demand manufacturing of clinical-quality biopharmaceuticals. Nature Biotechnology 36, 988 (2018).

70. Needham, B. D. et al. Modulating the innate immune response by combinatorial engineering of endotoxin. Proceedings of the National Academy of Sciences 110, 1464-1469 (2013).

71. Wilding, K. M. et al. Endotoxin-Free E. coli-Based Cell-Free Protein Synthesis: Pre-Expression Endotoxin Removal Approaches for on-Demand Cancer Therapeutic Production. Biotechnology journal 14, 1800271 (2019).

72. Schreiner, R., Schnabel, E. & Wieland, F. Novel N-glycosylation in eukaryotes: laminin contains the linkage unit beta-glucosylasparagine. The Journal of cell biology 124, 1071-1081 (1994).

73. Kong, Y. et al. N-Glycosyltransferase from Aggregatibacter aphrophilus synthesizes glycopeptides with relaxed nucleotide-activated sugar donor selectivity. Carbohydrate Research 462, 7-12 (2018).

74. Gibson, D. G. et al. Enzymatic assembly of DNA molecules up to several hundred kilobases. Nature Methods 6, 343-345 (2009).

75. Ollis, A. A., Zhang, S., Fisher, A. C. & DeLisa, M.P. Engineered oligosaccharyltransferases with greatly relaxed acceptor-site specificity. Nature Chemical Biology 10, 816-822 (2014).

76. Espah Borujeni, A., Channarasappa, A. S. & Salis, H. M. Translation rate is controlled by coupled trade-offs between site accessibility, selective RNA unfolding and sliding at upstream standby sites. Nucleic Acids Research 42, 2646-2659 (2014).

77. Valentine, Jenny L. et al. Immunization with Outer Membrane Vesicles Displaying Designer Glycotopes Yields Class-Switched, Glycan-Specific Antibodies. Cell Chemical Biology 23, 655-665 (2016).

78. Kim, D. M. & Swartz, J. R. Efficient production of a bioactive, multiple disulfide-bonded protein using modified extracts of Escherichia coli. Biotechnology and bioengineering 85, 122-129 (2004).

79. Baba, T. et al. Construction of Escherichia coli K-12 in-frame, single-gene knockout mutants: the Keio collection. Molecular systems biology 2, 2006.0008-2006.0008 (2006).

80. St-Pierre, F. et al. One-Step Cloning and Chromosomal Integration of DNA. ACS synthetic biology 2, 537-541 (2013).

The contents of the afore-cited non-patent references are incorporated herein by reference in their entireties.

### Example 2

### Method for Incorporation of Non-Standard Sugars in Living E. coli Cells

### Overview

We incorporated non-standard (azido) variants of sialic acid in living E. coli at the end of an N-linked trisaccharide (Asn-Glc-Gal-Sia) using pathways described above for the GlycoPRIME methods. This approach can be used to provide both a general modification strategy for small therapeutics (PEGylation, etc) as well as an approach for the production of allergen vaccines by incorporating specific sialic acids known to create tolerogenic responses with siglecs and galectins. This is interesting compared to the state of the art because this provides the first instance of incorporating a non-standard (or click-able) glycan for use in protein therapeutics in living E. coli. As such, it could be easier than current methods either in mammalian cells or enzymatic in vitro methods to install non-standard sialic acids. As described below, we have applied the minimal sialic acid glycan pathways developed using GlycoPRIME to the production of recombinant proteins with clickable sialic acids in E. coli. Our data demonstrates the incorporation and these azido-sialic acids into the Im7-6 model protein and Fc-6.

In contrast to classical immunogenic vaccines, tolerogenic vaccines are designed to induce long-term, antigen-specific, inhibitory memory that prevents an inflammatory immune response to a benign substance such as an allergen or target of an autoimmune disorders¹. There is recent evidence that the binding of siglecs to sialic acids on cells and antigens may play an important role in tolerogenic responses mediated by immune cells (particularly dendritic and regulatory T-cells)^{2, 3}. There is further evidence that siglec-sialic acid interactions can be amplified and tuned using chemically modified sialic acids⁴⁻⁹. Therefore, the association of sialic acids and, especially, chemically modified sialic acids with allergens or proteins targeted by autoimmunity presents a promising therapeutic strategy to treat allergies or autoimmune disorders^{7, 10-12}. The use of metabolic labeling to incorporate sialic acids with alkyne moieties into cell-surface proteins for further chemical modification using click chemistry¹³ to modulate siglec interactions has also been shown⁷. Methods to install azido-sialic acids in bacteria using pathways developed in GlycoPRIME could provide new routes to these tolerogenic vaccines.

Once produced in our system, these clickable sialic acids could be further functionalized with a variety of high-affinity and selective ligands for siglecs to produce tolerogenic vaccines. Because it takes place in bacteria which have lower production costs and can be more easily engineered, this system would be complementary to other mammalian-based metabolic labeling system. In theory, the only required modification to system used to collect this preliminary data to achieve this goal is the substitution of the target protein plasmid with a plasmid encoding a protein for which tolerance induction is desired fused to a repeating region of GlycTags targeted by ApNGT, similar to the constructs described in a previous study¹⁴.

In addition to allowing the modulation of siglec binding, the azido-sialic acid glycans could also serve as a general chemical handle for the attachment of polyethylene glycol (PEG) to small therapeutics (such as GM-CSF) to increase their circulatory half-life or the attachment of a chemotherapeutic “warhead” to a short chain antibody fragment or nanobody to enable precise targeting and destruction of cancer cells. While there are other methods to install a chemical handle onto proteins in bacteria such as the incorporation of a non-standard amino acid or previously reported GlycoPEGylation strategies^{15, 16}, this method does have the advantage of not requiring the use of an orthogonal translation system or expensive non-natural activated sugar donors or purified enzymes (as GlycoPEGylation does).

### Method

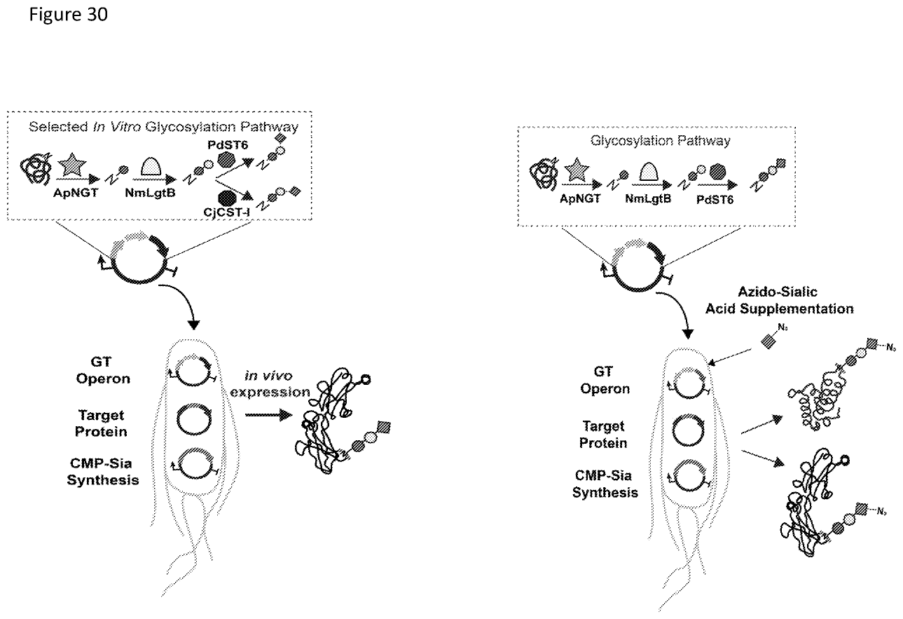

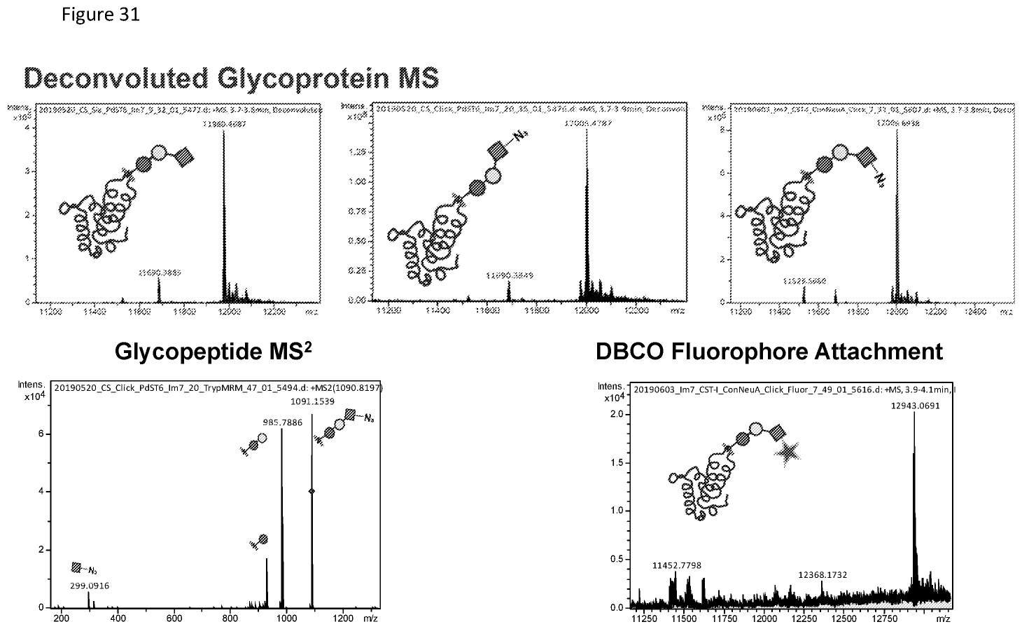

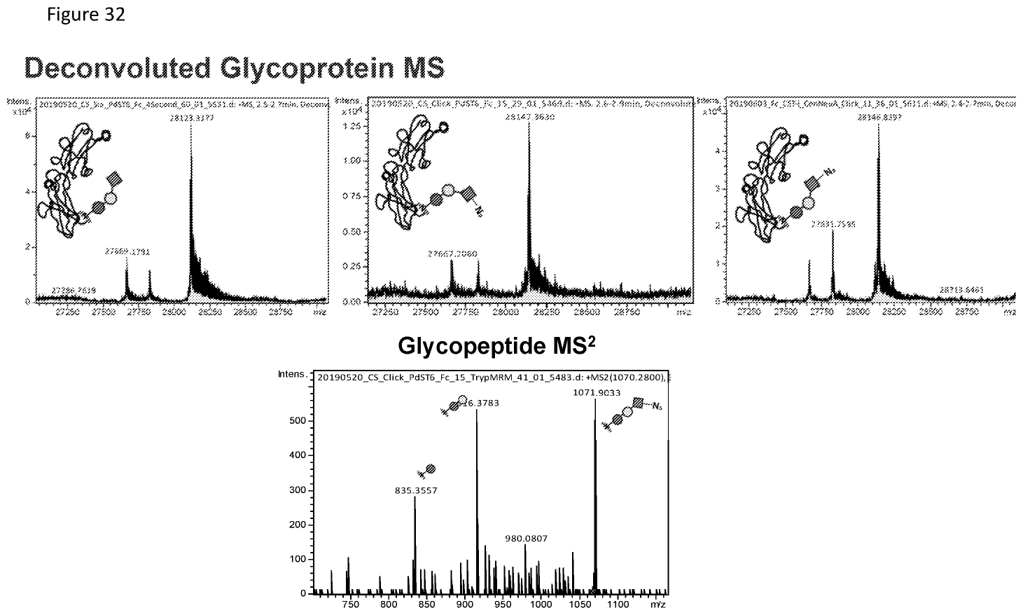

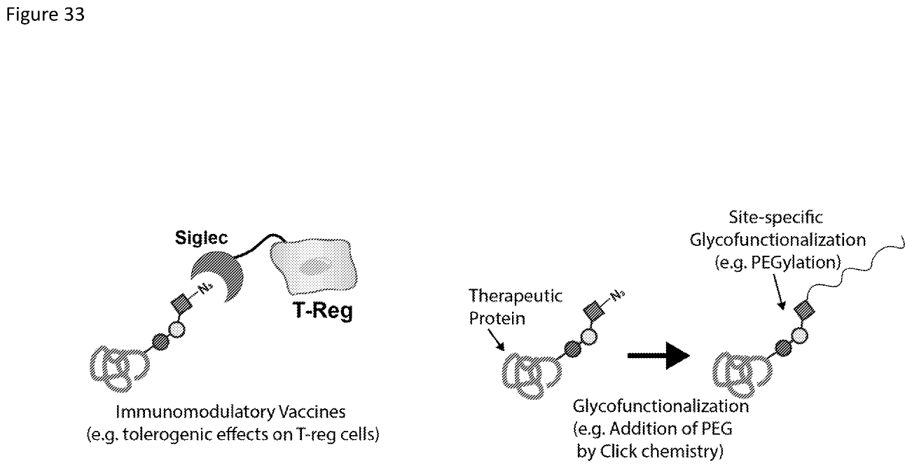

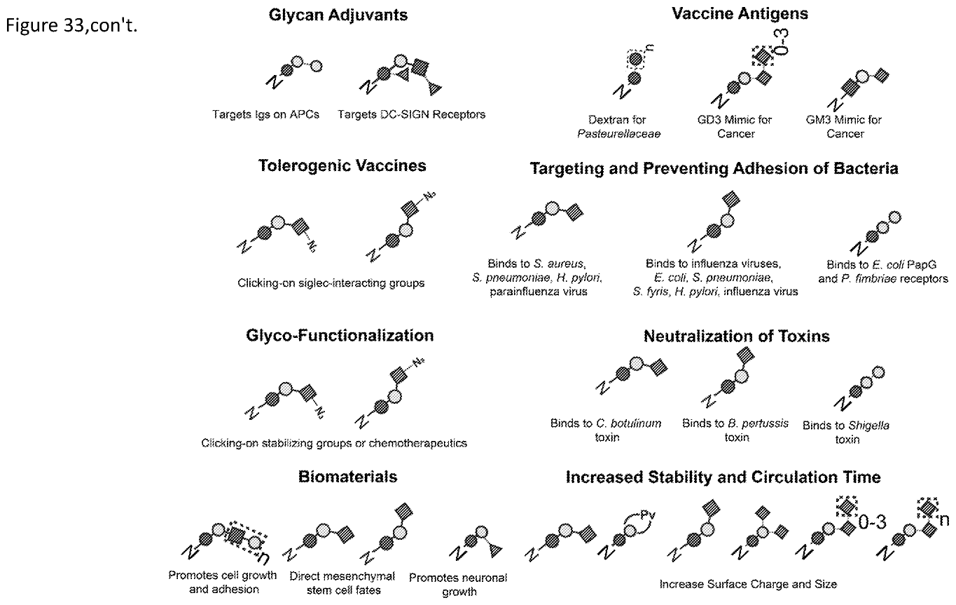

The same three-enzyme pathways implemented in the in vivo method described above in Example 1, and illustrated in FIG. 4 (ApNGT, LgtB, and CST-1 or Pd2ST6) were used in this Example. Briefly, an E. coli culture in which the bacteria were transformed with three plasmids carrying three glycosyltransferases, a CMP-Sia synthase, and a target protein with an optimized pepetide acceptor sequcnes for NGT was supplemented with an azido sialic acid (deoxy C-9; C-5 may also be substituted) synthetic sugar (substituted at the 9 position, purchased from CarboSynth). See FIG. 30. As shown in FIGS. 31 and 32 non-standard sugars were incorporated into glycoproteins; bacteria took up azido sugar and incorporated it into glycoproteins as a trisaccharide Asn-Glc-Gal-azido-Sia using the implemented pathway at very high efficiency (nearly 100%, see MS spectra at FIGS. 31 and 32). In the Figures, intact protein MS data and glycopeotide MS/MS data conclusively show the efficient incorporation of azido sialic acid (distinguished from standard sialic aicds by a 24 Da mass difference) by supplementation of azido-sialic acid into the media with E.coli containing the same three plasmid system that was described for GlycoPRIME, above. Thus, NanT sialic acid transporter, CMP-Sia synthase, and PdST6 as well as CST-I Sia Ts all accepted the non-standard sugar. Because there is no natural sialic acid in the system, non-specific incorporation is not a serious concern and was not observed in the spectra. Thus, C9-azido sialic acids can be attached with 2,6 and 2,3 linkages. Bacteria took up azido sugar and incorporated it into glycoproteins as a trisaccharide Asn-Glc-Gal-azido-Sia using the implemented pathway at very high efficiency. This is the first instance of incorporating azido sugar monomers into recombinantly expressed glycoproteins in a bacterial host using a recombinantly expressed protein glycosylation pathway.

The table below provides exemplary, non-limiting targets for allergen gene desing using the compositions and methods disclosed herein.

| Name | Abbreviation | Uniprot | Allergen | Disulfides? | PDB | Reason |
|---|---|---|---|---|---|---|
| Pollen allergen | Betv1 | O23748_BETPN | Birch | No | 1BV1 | ALK |
| from Betula | | | pollen | | | defined |
| pendula | | | | | | |
| (European | | | | | | |
| White Birch) | | | | | | |
| Blo t 1 allergen | | CYSP_BLOTA | | | 5JT8 | A common |
| | | | | | | allergen on |
| | | | | | | Protein |
| | | | | | | Data Bank |
| | | | | | | (PDB) |
| Blomia Dust | Blo t 5 | ALL5_BLOTA | Dust | No | 2JRK | A common |
| mite allergen 5 | | | Mite | | | allergen on |
| | | | | | | PDB |
| Blomia Dust | Blo t 21 | ALL21_BLOTA | Dust | No | 2LM9 | A common |
| mite allergen 21 | | | Mite | | | allergen on |
| | | | | | | PDB |
| Dust Mite | Derp1 | | | | 1XKG, | A common |
| Allergen Der p 1 | | | | | 3F5V | allergen on |
| | | | | | | PDB |
| Mite group 2 | Derp2 | ALL2_DERPT | Dust | Yes | 1a9v | ALK |
| allergen Der p 2 | | | Mite | | | defined |
| Dust Mite | Derp5 | | | | 3MQ1 | A common |
| Allergen Der p 5 | | | | | | allergen on |
| | | | | | | PDB |
| Der p 7 fusion | | | | | 3H4Z | A common |
| protein | | | | | | allergen on |
| | | | | | | PDB |
| mite allergen | | | | | 3D6S, | A common |
| Der f 1 | | | | | 5VPK | allergen on |
| | | | | | | PDB |
| Pollen Allergen | phlp5 | MPAP5_PHLPR | Timothy | | 2M64 | ALK |
| Phl p 5 | | | Grass | | | defined |
| (crystallized | | | Pollen | | | |
| version); dimer | | | | | | |
| Soybean | | | | | 2K7H | A common |
| allergen Gly m4 | | | | | | allergen on |
| | | | | | | PDB |
| allergen arah6 | | | | | 1W2Q | A common |
| from peanut | | | | | | allergen on |
| (Arachis | | | | | | PDB |
| hypogaea) | | | | | | |

In some embodiments, allergens or autoimmune targets that have previously been expressed in E. coli and are nto disulfide bonded are selected. Additionally or alternatively, in some embodiments, “glycoModules,” with, for example, 1, 5, or 10 repeated acceptor sequences are employed. In some embodiments, these multiple sequences are closely packed, while still ensuring good modification (e.g., native acceptors on COK aor HMW1 protiens or GlycoSCORES).

In some embodiments, just a non-natural sugar is added. By way of example, but not by way of limitation, just glucose is added to the cell-free lysacte (which may be substituted with precise sugar donor synthases) and the monosaccharides can be charged onto a surgar donor.

### References for Example 2

1. Mannie, M. D. & Curtis, A. D., 2nd Tolerogenic vaccines for Multiple sclerosis. Human vaccines & immunotherapeutics 9, 1032-1038 (2013).

2. Švajger, U. & Rožman, P. Induction of Tolerogenic Dendritic Cells by Endogenous Biomolecules: An Update. Frontiers in immunology 9, 2482-2482 (2018).

3. Lubbers, J., Rodriguez, E. & van Kooyk, Y. Modulation of Immune Tolerance via Siglec-Sialic Acid Interactions. Frontiers in immunology 9, 2807-2807 (2018).

4. Rillahan, C. D., Schwartz, E., McBride, R., Fokin, V. V. & Paulson, J. C. Click and Pick: Identification of Sialoside Analogues for Siglec-Based Cell Targeting. Angewandte Chemie International Edition 51, 11014-11018 (2012).

5. Spence, S. et al. Targeting Siglecs with a sialic acid-decorated nanoparticle abrogates inflammation. Science Translational Medicine 7, 303ra140-303ra140 (2015).

6. Prescher, H., Schweizer, A., Kuhfeldt, E., Nitschke, L. & Brossmer, R. Discovery of Multifold Modified Sialosides as Human CD22/Siglec-2 Ligands with Nanomolar Activity on B-Cells. ACS Chemical Biology 9, 1444-1450 (2014).

7. Bull, C. et al. Steering Siglec-Sialic Acid Interactions on Living Cells using Bioorthogonal Chemistry. Angewandte Chemie International Edition 56, 3309-3313 (2017).

8. Bull, C., Heise, T., Adema, G.J. & Boltje, T.J. Sialic Acid Mimetics to Target the Sialic Acid-Siglec Axis. Trends in Biochemical Sciences 41, 519-531 (2016).

9. Abdu-Allah, H. H. M. et al. CD22-Antagonists with nanomolar potency: The synergistic effect of hydrophobic groups at C-2 and C-9 of sialic acid scaffold. Bioorganic & Medicinal Chemistry 19, 1966-1971 (2011).

10. Perdicchio, M. et al. Sialic acid-modified antigens impose tolerance via inhibition of T-cell proliferation and de novo induction of regulatory T cells. Proceedings of the National Academy of Sciences 113, 3329-3334 (2016).

11. Pang, L., Macauley, M. S., Arlian, B. M., Nycholat, C. M. & Paulson, J. C. Encapsulating an Immunosuppressant Enhances Tolerance Induction by Siglec-Engaging Tolerogenic Liposomes. Chembiochem: a European journal of chemical biology 18, 1226-1233 (2017).

12. Orgel, K. A. et al. Exploiting CD22 on antigen-specific B cells to prevent allergy to the major peanut allergen Ara h 2. Journal of Allergy and Clinical Immunology 139, 366-369.e362 (2017).

13. Kolb, H. C., Finn, M. & Sharpless, K. B. Click chemistry: diverse chemical function from a few good reactions. Angewandte Chemie International Edition 40, 2004-2021 (2001).

14. Mathiesen, C. B. K. et al. Genetically engineered cell factories produce glycoengineered vaccines that target antigen-presenting cells and reduce antigen-specific T-cell reactivity. Journal of Allergy and Clinical Immunology 142, 1983-1987 (2018).

15. DeFrees, S. et al. GlycoPEGylation of recombinant therapeutic proteins produced in Escherichia coli. Glycobiology 16, 833-843 (2006).

16. Henderson, G. E., Isett, K. D. & Gerngross, T. U. Site-Specific Modification of Recombinant Proteins: A Novel Platform for Modifying Glycoproteins Expressed in E. coli. Bioconjugate chemistry 22, 903-912 (2011).

17. Santos da Silva E, Asam C, Lackner P, et al. Allergens of Blomia tropicalis: An Overview of Recombinant Molecules. Int Arch Allergy Immunol. 2017;172(4):203-214. doi:10.1159/000464325

18. Derewenda, U., Li, J., Derewenda, Z., Dauter, Z., Mueller, G. A., Rule, G. S. & Benjamin, D.C. The crystal structure of a major dust mite allergen Der p 2, and its biological implications. J Mol Biol 318, 189-197 (2002).

19. Marković-Housley, Z., Degano, M., Lamba, D., von Roepenack-Lahaye, E., Clemens, S., Susani, M., Ferreira, F., Scheiner, O. & Breiteneder, H. Crystal Structure of a Hypoallergenic Isoform of the Major Birch Pollen Allergen Bet v 1 and its Likely Biological Function as a Plant Steroid Carrier. Journal of Molecular Biology 325, 123-133 (2003).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A cell-free system for glycosylating a peptide or polypeptide sequence in vitro, the peptide or polypeptide sequence comprising an asparagine residue and the system comprising as components:
(i) a glycosyltransferase which is an N-glycosyltransferase (NGT) that catalyzes transfer to an amino group of the asparagine residue a monosaccharide to provide an N-linked glycan, or an expression vector that expresses the NGT in a cell-free protein synthesis (CFPS) reaction mixture;
(ii) a glycosylation mixture comprising a monosaccharide donor, optionally a monosaccharide;
wherein the peptide or polypeptide sequence is glycosylated in the glycosylation mixture in vitro to provide a peptide or polypeptide sequence comprising the N-linked glycan.

2. The system of claim 1, further comprising as a component:
(iii) a second glycosyltransferase that catalyzes transfer to the N-linked glycan a monosaccharide, or an expression vector that expresses the second glycosyltransferase in a cell-free protein synthesis (CFPS) reaction mixture;
wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan is glycosylated with one or more moieties selected from Glc, Gal, GalNAc, GlcNAc, pyruvate, Fuc, Sia, and a non-natural sugar.

3. The system of claim 2 further comprising as a component:
(iv) a third glycosyltransferase that catalyzes transfer to the N-linked glycan a monosaccharide, or an expression vector that expresses the third glycosyltransferase in a cell-free protein synthesis (CFPS) reaction mixture;
wherein the glycosylation mixture comprises a Glc donor, a Gal donor, a GalNAc donor, a GlcNAc donor, a pyruvate donor, a fucose donor, a sialic acid donor, or a mixture thereof, and wherein the N-linked glycan further is glycosylated with one or more moieties selected from Glc, Gal, GalNAc, GlcNAc, pyruvate, Fuc, Sia, and azido-Sia.

4. The system of claim 1, wherein the system comprises a cell-free protein synthesis (CFPS) reaction mixture and one or more of the first glycosyltransferase, the second glycosyltransferase, and the third glycosyltransferase are present or expressed in the CFPS reaction mixture.

5. The system of claim 1, wherein the system comprises one or more cell-free protein synthesis (CFPS) reaction mixtures and one or more of the first glycosyltransferase, the second glycosyltransferase, and the third glycosyltransferase are present or expressed in the CFPS reaction mixtures and the one or more CFPS reaction mixtures are combined to provide the system.

6. The system of claim 1, further comprising the peptide or polypeptide sequence or an expression vector that expresses the peptide or polypeptide sequence.

7. The system of claim 1, further comprising a prokaryotic CFPS reaction mixture.

8. The system of claim 1, further comprising a prokaryotic CFPS reaction mixture comprising a lysate prepared from Escherichia coli.

9. The system of claim 1, wherein the glycosyltransferase is a bacterial N-linked glycosyltransferase (NGT) selected from the group consisting of Actinobacillus pleuropneumoniae (ApNGT), Escherichia coli NGT (EcNGT), Haemophilus influenza NGT (HiNGT), Mannheimia haemolytica NGT (MhNGT), Haemophilus dureyi NGT (HdNGT), Bibersteinia trehalosi NGT (BtNGT), Aggregatibacter aphrophilus NGT (AaNGT), Yersinia enterocolitica NGT (YeNGT), Yersinia pestis NGT (YpNGT), and Kingella kingae NGT (KkNGT) or a modified form thereof.

10. The system of claim 1, wherein the glycosyltransferase is a bacterial N-linked glycosyltransferase (NGT) having the amino acid sequence of any of SEQ ID NOs:1, 3, 5, 7, 9, 11, 13, 15, 17, or 19 or having a least 50%, 60%, 70%, 80%, 90%, 95%, 96%, 97%, 98%, or 99% sequence identity to any of SEQ ID NOs:1, 3, 5, 7, 9, 11, 13, 15, 17, or 19, or the first glycosyltransferase is a modified bacterial N-linked glycosyltransferase (NGT) having the amino acid sequence of any of SEQ ID NOs:2, 4, 6, 8, 10, 12, 14, 16, 18, or 20, or having a least 50%, 60%, 70%, 80%, 90%, 95%, 96%, 97%, 98%, or 99% sequence identity to any of SEQ ID NOs:2, 4, 6, 8, 10, 12, 14, 16, 18, or 20.

11. The system of claim 2, wherein the second glycosyltransferases is an αl-6 glucosyltransferase, a β1-4 galactosyltransferase, or a β1-3 N-acetylgalactosamine transferase selected from the group consisting of Actinobacillus pleuropneumoniae α1-6 glucosyltransferase (Apα1-6), Neisseria gonorrhoeae β1-4 galactosyltransferase LgtB (NgLGtB), Neisseria meningitidis β1-4 galactosyltransferase LgtB (NmLGtB), and Bacteriodes fragilis β1-3 N-acetylgalactosamine transferase (BfGalNAcT).

12. The system of claim 3, wherein the third glycosyltransferase is a β1-3 N-acetylglucosamine transferase, a pyruvyltransferase, an α1-3 fucosyltransferase, an α1-2 fucosyltransferase, an α1-4 galactosyltransferase, an α1-3 galactosyltransferase, an α2-6 sialyltransferase, an α2-3,6 sialyltransferase, an α2-3 sialyltransferase, or an α2-3,8 sialyltransferase selected from the group consisting of Neisseria gonorrhoeae β1-3 N-acetylglucosamine transferase (NgLgtA), Schizosaccharomyces pombe pyruvyltransferase (SpPvg1), Helicobacter pylori α1-3 fucosyltransferase (HpFutA), Helicobacter pylori α1-2 fucosyltransferase (HpFutC), Neisseria meningitidis α1-4 galactosyltransferase (NmLgtC), Bos taurus α1-3 galactosyltransferase (BtGGTA), Homo sapiens α2-6 sialyltransferase (HsSIAT1), Photobacterium damselae α2-6 sialyltransferase (PdST6), Photobacterium leiognathid α2-6 sialyltransferase (P1ST6), Pasteurella multocida α2-3,6 sialyltransferase (PmST3,6), Vibrio sp JT-FAJ-16 α2-3 sialyltransferase (VsST3), Photobacterium phosphoreum α2-3 sialyltransferase (PpST3), Campylobacter jejuni α2-3 sialyltransferase (CjCST-I), and Campylobacter jejuni a2-3,8 sialyltransferase (CjCST-II).

13. The system of claim 1, wherein one or more components of the system are in a freeze-dried form.

14.-26. (canceled)

27. A peptide or polypeptide sequence comprising an N-linked glycan, the N-linked glycan comprising a moiety selected from the group consisting of sialylated forms of lactose, fucosylated forms of lactose, sialylated forms of LacNAc (lactose-(poly)LacNAc), fucosylated forms of LacNAc (lactose-(poly)LacNAc), pyruvylated lactose, pyruvylated LacNAc (lactose-(poly)LacNAc), glucose, polyα1,6-linked glucose, glucose modified with β1,3 GalNAc, lactose, lactose modified with (poly)LacNAc (lactose-(poly)LacNAc), lactose modified with α1,4 galactose, lactose modified with oligo-sialic acid and an αGal epitope.

28. A modified bacterial cell that comprises or expresses one or more components of the system of claim 1.

29. A lysate prepared from the modified cell of claim 28 suitable for use in a cell-free protein synthesis (CFPS) reaction.

30. A method for preparing a glycosylated peptide or polypeptide sequence, the method comprising culturing the modified bacterial cell of claim 28, wherein the modified cell comprises or expresses a peptide or polypeptide sequence, and an N-linked glycosyltransferase.

31. A method for preparing a glycosylated peptide or polypeptide sequence in vitro, the method comprising reacting a peptide or polypeptide sequence comprising an asparagine residue in a glycosylation mixture comprising a monosaccharide donor with a glycosyltransferase which is a N-glycosyltransferase (NGT) that catalyzes transfer of the monosaccharide from the monosaccharide donor to an amino group of the asparagine residue to provide an N-linked glycan, wherein the peptide or polypeptide sequence is glycosylated in the glycosylation mixture in vitro to provide a peptide or polypeptide sequence comprising the N-linked glycan.

32. A system for preparing a glycosylated peptide or polypeptide sequence, the peptide or polypeptide sequence comprising an asparagine residue and the system comprising as components:
(i) a modified bacterial cell, optionally wherein the bacterial cell is modified to express an exogenous glycosyltransferase which is an N-glycosyltransferase (NGT) that catalyzes transfer to an amino group of the asparagine residue a monosaccharide to provide an N-linked glycan, or an expression vector that expresses the NGT in a cell-free protein synthesis (CFPS) reaction mixture;
(ii) a glycosylation mixture comprising a non-natural sugar donor, optionally added to media for growing the modified bacterial cell;
wherein the peptide or polypeptide sequence is glycosylated in the modified bacterial cell to provide the peptide or polypeptide sequence comprising the non-natural sugar.

33. A method for preparing a preparing a glycosylated peptide or polypeptide sequence, the method comprising expressing the peptide or polypeptide sequence in the modified bacterial cell of the system of claim 32, and glycosylating the expressed peptide or polypeptide sequence.
